SEQUENCE LISTING (109 sequences)

SEQ ID NO: 1 - protein, 509 aa, Artificial
  EPOR mammalian
MXXXXXXXWPXVXXLCLLLAGAXWAXXPXXPDXKFESKAALLAXRGXEELLCFTXRLEDLVCFWEEAXXXGXXXXXYSFSYQLEXEXXKXCXLHQXPTXRGXXRFWCSLPTADTSSFVPLELXVTXXXSGXPRYHRXIHINEVVLLDXPXGLXARXAXEXXHVVLRWLPPPXXPMXXXIRYEVXXSXXNXAGXXQRVEXLXGRTECVLSNLRGXTRYTFXVRARMAEPSFXGFWSAWSEPXSLLTXSDLDPLILTLSLILVXIXXLLXVLALLSHRRXLXQKIWPGIPSPEXEFEGLFTTHKGNFQLWLXQXDGCLWWSPXXXFXEDPPAXLEVLSEXXWXXXQAXXXXXXDXGXLLEPVGSEXAXDTYLVLDXWLLPRXPXSEXLXXPGXXXDXXXMDEXSEXSXCXSXLAXKPXPEGXSXXSFEYTILDPSSXLLXPXXLXXELPPTPPHLKYLYLVVSDSGISTDYSSGXSQXXXGXXSXGPYSXPYENSLXPXXEPXXPXYVXCS

SEQ ID NO: 2 - protein, 508 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 3 - protein, 507 aa, Mus musculus
MDKLRVPLWPRVGPLCLLLAGAAWAPSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSHRRTLQQKIWPGIPSPESEFEGLFTTHKGNFQLWLLQRDGCLWWSPGSSFPEDPPAHLEVLSEPRWAVTQAGDPGADDEGPLLEPVGSEHAQDTYLVLDKWLLPRTPCSENLSGPGGSVDPVTMDEASETSSCPSDLASKPRPEGTSPSSFEYTILDPSSQLLCPRALPPELPPTPPHLKYLYLVVSDSGISTDYSSGGSQGVHGDSSDGPYSHPYENSLVPDSEPLHPGYVACS

SEQ ID NO: 4 - protein, 507 aa, Rattus norvegicus
MDQLRVARWPRVSPLCLLLAGAAWASSPSLPDPKFESKAALLASRGSEELLCFTQRLEDLVCFWEEAANSGMGFNYSFSYQLEGESRKSCRLHQAPTVRGSMRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSHRRALRQKIWPGIPSPENEFEGLFTTHKGNFQLWLLQRDGCLWWSPSSPFPEDPPAHLEVLSERRWGVTQAGDAGAEDKGPLLEPVGSERAQDTYLVLDEWLLPRCPCSENLSGPGDSVDPATMDEGSETSSCPSDLASKPRPEGTSPSSFEYTILDPSSKLLCPRALPPELPPTPPHLKYLYLVVSDSGISTDYSSGGSQGVHGDSSDGPYSHPYENSLVPDTEPLRPSYVACS

SEQ ID NO: 5 - protein, 509 aa, Sus scrofa
MYHFGATLWPGVGSLCLLLAGATWAPSPNSPDAKFESKAALLAARGPEELLCFTERLEDLVCFWEEAGSAGVGPEDYSFSYQLEGEPWKPCHLHQGPTARGSVRFWCSLPTADTSSFVPLELRVTEVSSGAPRYHRIIHINEVVLLDPPAGLLARRAEESGHVVLRWLPPPGAPMASLIRYEVNISTENAAGGVQRVEILDGRTECVLSNLRGGTRYTFMVRARMAEPSFGGFWSAWSEPASLLTASDLDPLILTLSLILVLILLLLAVLALLSHRRTLKQKIWPGIPSPEGEFEGLFTTHKGNFQLWLYQTDGCLWWSPCTPFAEDPPAPLEVLSERCWGVTQAVEPAADDEGSLLEPVGSEHARDTYLVLDKWLLPRRPASEDLPQPGGDLDMAAMDEASEASFCSSALALKPGPEGASAASFEYTILDPSSQLLRPRALPAELPPTPPHLKYLYLVVSDSGISTDYSSGGSQETQGGSSSGPYSNPYENSLVPAPEPSPPNYVTCS

SEQ ID NO: 6 - protein, 194 aa, Artificial
  EPO mammalian
MGXXXXXXXXXLXLSXXXXPLGXPVXXAPXRLXCDSRVLERYXLXAXEAENXTXGCXEXXXXXENITVPDTKVNFYXWKRXXVXXQAXEVWQGLXLLSEAXXXXQAXXXNXSQPXEXLXLHXDKAXSXLRSXTXLLRXLGAQXEXXXXPXXXXXXAPLRXXTXDXXXKLFRXYXNFLRGKLXLYTGEXCRXGDR

SEQ ID NO: 7 - protein, 193 aa, Homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 8 - protein, 192 aa, Macaca mulatta
MGVHECPAWLWLLLSLVSLPLGLPVPGAPPRLVCDSRVLERYLLEAKEAENVTMGCSESCSLNENITVPDTKVNFYAWKRIEVGQQAVEVWQGLALLSEAVLRGQAVLANSSQPFEPLQLHMDKAISGLRSITTLLRALGAQEAISLPDAASAAPLRTITADTFCKLFRVYSNFLRGKLKLYTGEACRRGDR

SEQ ID NO: 9 - protein, 192 aa, Macaca fascicularis
MGVHECPAWLWLLLSLVSLPLGLPVPGAPPRLICDSRVLERYLLEAKEAENVTMGCSESCSLNENITVPDTKVNFYAWKRMEVGQQAVEVWQGLALLSEAVLRGQAVLANSSQPFEPLQLHMDKAISGLRSITTLLRALGAQEAISLPDAASAAPLRTITADTFCKLFRVYSNFLRGKLKLYTGEACRRGDR

SEQ ID NO: 10 - protein, 192 aa, Mus musculus
MGVPERPTLLLLLSLLLIPLGLPVLCAPPRLICDSRVLERYILEAKEAENVTMGCAEGPRLSENITVPDTKVNFYAWKRMEVEEQAIEVWQGLSLLSEAILQAQALLANSSQPPETLQLHIDKAISGLRSLTSLLRVLGAQKELMSPPDTTPPAPLRTLTVDTFCKLFRVYANFLRGKLKLYTGEVCRRGDR

SEQ ID NO: 11 - protein, 192 aa, Rattus norvegicus
MGVPERPTLLLLLSLLLIPLGLPVLCAPPRLICDSRVLERYILEAKEAENVTMGCAEGPRLSENITVPDTKVNFYAWKRMKVEEQAVEVWQGLSLLSEAILQAQALQANSSQPPESLQLHIDKAISGLRSLTSLLRVLGAQKELMSPPDATQAAPLRTLTADTFCKLFRVYSNFLRGKLKLYTGEACRRGDR

SEQ ID NO: 12 - protein, 192 aa, Bos taurus
MGARDCTPLLMLSFLLFPLGFPVLGAPARLICDSRVLERYILEAREAENATMGCAEGCSFNENITVPDTKVNFYAWKRMEVQQQALEVWQGLALLSEAILRGQALLANASQPCEALRLHVDKAVSGLRSLTSLLRALGAQKEAISLPDATPSAAPLRAFTVDALSKLFRIYSNFLRGKLTLYTGEACRRGDR

SEQ ID NO: 13 - protein, 194 aa, Ovis aries
MGARDCTPLLLLLLSFLLFPLGLPVLGAPPRLICDSRVLERYILEAREAENATMGCAEGCSFSENITVPDTKVNFYAWKRMEVQQQALEVWQGLALLSEAIFRGQALLANASQPCEALRLHVDKAVSGLRSLTSLLRALGAQKEAIPLPDATPSAAPLRIFTVDALSKLFRIYSNFLRGKLTLYTGEACRRGDR

SEQ ID NO: 14 - protein, 192 aa, Felis catus
MGSCECPALLLLLSLLLLPLGLPVLGAPPRLICDSRVLERYILGAREAENVTMGCAEGCSFSENITVPDTKVNFYTWKRMDVGQQAVEVWQGLALLSEAILRGQALLANSSQPSETLQLHVDKAVSSLRSLTSLLRALGAQKEATSLPEATSAAPLRTFTVDTLCKLFRIYSNFLRGKLTLYTGEACRRGDR

SEQ ID NO: 15 - DNA, 942 bp, Artificial
  PIM1 cDNA mammalian
atgctcytgtccaaratcaactcsctkgcccacctgcgcrcmgcsccytgcarcgacctgcacgccamcaagctggcgccsggcaargagaaggagcccctggagtcgcagtaccaggtgggcccgctvytgggcagyggyggcttcggctcggtstactcdggcatccgbgtckccgacaacttgccggtggccatyaarcacgtggagaaggaccggatttccgaytggggrgarctgccyaayggcacycgagtgcccatggargtggtyctgytgaagaaggtgagctcggryttctcsggcgtcattmgrctyctggactggttygagaggcccgayagtttcgtsytgatcctggagaggccsgarccsgtgcaagayctcttcgacttyatcacsgarmgrggrgcyctvcargaggasctsgcccgvrgmttcttctggcaggtrctggaggcsgtgcgrcaytgccacractgcggggtkctycaccgcgacatcaaggacgaraacatcythatcgacctsarycgcggcgarmtcaarctcatcgacttcgggtcgggggcgctgctcaargacacmgtctacacggacttygayggracccgagtgtayagyccyccagagtggatycgctaycatcgctaccayggcaggtcggcngchgtytggtcyctkgggatcctgctstatgayatgktskkhggwgawwtkmmnywyswsmwyrwbswvrrkrkyrtcwvrksmmarkykywyktyaksmwvmbksyywyytcwgartgtcarcayctyattaratggtgcytgkccytgagaccrtcrgaymggccmwccttygaagaaatccrgaaccatccvtggatgcargrygwyctcctgccccaggmmrchkcygagatycayctscacagyctgtcrccrksryccagcaartag

SEQ ID NO: 16 - DNA, 942 bp, Homo sapiens
atgctcttgtccaaaatcaactcgcttgcccacctgcgcgccgcgccctgcaacgacctgcacgccaccaagctggcgcccggcaaggagaaggagcccctggagtcgcagtaccaggtgggcccgctactgggcagcggcggcttcggctcggtctactcaggcatccgcgtctccgacaacttgccggtggccatcaaacacgtggagaaggaccggatttccgactggggagagctgcctaatggcactcgagtgcccatggaagtggtcctgctgaagaaggtgagctcgggtttctccggcgtcattaggctcctggactggttcgagaggcccgacagtttcgtcctgatcctggagaggcccgagccggtgcaagatctcttcgacttcatcacggaaaggggagccctgcaagaggagctcgcccgcagcttcttctggcaggtgctggaggccgtgcggcactgccacaactgcggggtgctccaccgcgacatcaaggacgaaaacatccttatcgacctcaatcgcggcgagctcaagctcatcgacttcgggtcgggggcgctgctcaaagacaccgtctacacggacttcgatgggacccgagtgtatagccctccagagtggatccgctaccatcgctaccatggcaggtcggcggcagtctggtccctggggatcctgctgtatgatatggtgtgtggagatattcctttcgagcatgacgaagagatcatcaggggccaggttttcttcaggcagagggtctcttcagaatgtcagcatctcattagatggtgcttggccctgagaccatcagataggccaaccttcgaagaaatccagaaccatccatggatgcaagatgttctcctgccccaggaaactgctgagatccacctccacagcctgtcgccggggcccagcaaatag

SEQ ID NO: 17 - DNA, 942 bp, Pan troglodytes
atgctcttgtccaaaatcaactcgcttgcccacctgcgcgccgcgccctgcaacgacctgcacgccaccaagctggcgcccggcaaggagaaggagcccctggagtcgcagtaccaggtgggcccgctactgggcagcggcggcttcggctcggtctactcaggcatccgcgtctccgacaacttgccggtggccatcaaacacgtggagaaggaccggatttccgactggggagagctgcctaatggcactcgagtgcccatggaagtggtcctgctgaagaaggtgagctcgggtttctccggcgtcattaggctcctggactggttcgagaggcccgacagtttcgtcctgatcctggagaggcccgagccggtgcaagatctcttcgactttatcacggaaaggggggccctgcaagaggagctggcccgcagcttcttctggcaggtgctggaggccgtgcggcactgccacaactgcggggtgctccaccgcgacatcaaggacgaaaacatccttatcgacctcaatcgcggcgagctcaagctcatcgacttcgggtcgggggcgctgctcaaggacaccgtctacacggacttcgatgggacccgagtgtatagccctccagagtggatccgctaccatcgctaccatggcaggtcggcggcagtctggtccctggggatcctgctgtatgatatgttggtaggtgaattgaatcatctcatcatgctcagtggtgtctcatcaaaatctcttgtcatcatccttcctatttctgaatgtcagcatctcattagatggtgcttggccctgagaccatcagataggccaaccttcgaagaaatccagaaccatccatggatgcaagatgttctcctgccccaggaaactgctgagatccacctccacagcctgtcgccggggcccagcaaatag

SEQ ID NO: 18 - DNA, 942 bp, Felis catus
atgctcttgtccaaaatcaactcgcttgcccacctgcgcaccgcgccctgcaacgacctgcacgccaccaagctggcgcccggcaaggagaaggagcccctggagtcgcagtaccaggtgggcccgctactgggcagcggcggcttcggctcggtctactcaggcatccgggtcgccgacaacttgccggtggccatcaagcacgtggagaaggaccggatttccgattggggagagctgcccaatggcacccgagtgcccatggaggtggtcctgctgaagaaggtgagctcgggcttctccggcgtcattcggctcctggactggtttgagaggcccgacagtttcgtcttgatcctggagaggcccgagccggtgcaagacctcttcgactttatcacggaaaggggggctctgcaggaggagctggcccgcagcttcttctggcaggtgctggaggccgtgcggcactgccacaactgcggggtgctccaccgcgacatcaaggacgagaacatcctcatcgacctcaatcgcggcgagctcaagctcatcgacttcgggtcgggggcgctgctcaaggacaccgtctacacggacttcgacgggacccgagtgtatagtcccccagagtggatccgctaccatcgctaccatggcaggtcggccgccgtctggtctctggggatcctgctgtatgatatggtctgtggagatattccttttgagcatgatgaagagatcatcaggggccaagttttcttcaggcagagggtctcttcagagtgtcagcatctcattagatggtgcttggccctgagaccgtcagaccggccatccttcgaagaaatccagaaccatccctggatgcaagatgtcctcctgccccaggaaacagccgagatccatctgcacagcctgtcaccagggcccagcaaatag

SEQ ID NO: 19 - DNA, 942 bp, Bos taurus
atgctcttgtccaaaatcaactcgcttgcccacctgcgcgccgcgccctgcagcgacctgcacgccaccaagctggcgccgggcaaggagaaggagcccctggagtcgcagtaccaggtgggcccgctcctgggcagtggcggcttcggctcggtgtactcaggcatccgtgtcgccgacaacttgccggtggccatcaagcacgtggagaaggaccggatttccgactggggagagctgcctaatggcacccgagtgcccatggaagtggttctgctgaagaaggtgagctcgggcttctccggcgtcattaggctcctggactggttcgagaggcccgacagtttcgtcctgatcctggagaggccggagccggtgcaagacctcttcgactttatcacggaaaggggggctctgcaggaggagctggcccgcagcttcttctggcaggtactggaggcggtgcgacactgccacgactgcggggtgcttcaccgcgacatcaaggacgagaacatccttatcgacctcaatcgcggcgagctcaagctcatcgacttcgggtcgggggcgctgctcaaggacaccgtctacacggacttcgatgggacccgagtgtatagtcctccagagtggatccgctatcatcgctaccatggcaggtcggcagccgtctggtctctggggatcctgctgtatgacatggtgtgcggagatattccctttgagcacgatgaggagattgtcaggggccaagttttcttcaggcagcgggtctcctcagagtgtcaacatctcattagatggtgcttggccttgagaccatcagatcggccaaccttcgaagaaatccagaaccatccgtggatgcaagacgtcctcctgccccaggaaactgctgagatccatctccacagcctgtcgccagggcccagcaaatag

SEQ ID NO: 20 - DNA, 942 bp, Rattus norvegicus
atgctcttgtccaagatcaactccctggcccacctgcgcgcagccccttgcaacgacctgcacgccaacaagctggcgccgggcaaagagaaggagcccctggagtcgcagtaccaggtgggcccgctgttgggcagcggtggcttcggctcggtctactcgggcatccgcgtcgccgacaacttgccggtggccatcaagcacgtggagaaggaccggatttccgactggggggaactgcccaacggcacccgagtgcccatggaagtggtcctgctgaagaaggtgagctcgggcttctcgggcgtcattagacttctggactggttcgagaggcccgatagtttcgtgctgatcctggagaggcccgaacccgtgcaagacctcttcgacttcatcaccgagcgaggagccctccaggaggagctggcccggagcttcttctggcaggtgctggaggccgtgcggcattgccacaactgcggggttctccaccgcgacatcaaggacgagaacatcttaatcgacctgaaccgcggcgaactcaaactcatcgacttcgggtcgggggcgctgctcaaggacacagtctacacggactttgacggaacccgagtgtacagtcctccagagtggattcgctaccatcgctaccacggcaggtcggctgctgtttggtccctggggatcctgctctatgacatggtctgcggagatattccatttgagcacgacgaagagatcgtcaagggccaagtgtactttaggcaaagggtctcttcagaatgtcaacatcttattagatggtgcctgtccctgagaccatcggaccggccctcctttgaagaaatccagaaccatccgtggatgcaggatgttctcctgccccaggccaccgccgagattcatctgcacagcctgtcaccatcacccagcaaatag

SEQ ID NO: 21 - DNA, 942 bp, Mus musculus
atgctcctgtccaagatcaactccctggcccacctgcgcgccgcgccctgcaacgacctgcacgccaccaagctggcgccgggcaaagagaaggagcccctggagtcgcagtaccaggtgggcccgctgttgggcagcggtggcttcggctcggtctactctggcatccgcgtcgccgacaacttgccggtggccattaagcacgtggagaaggaccggatttccgattggggagaactgcccaatggcacccgagtgcccatggaagtggtcctgttgaagaaggtgagctcggacttctcgggcgtcattagacttctggactggttcgagaggcccgatagtttcgtgctgatcctggagaggcccgaaccggtgcaagacctcttcgactttatcaccgaacgaggagccctacaggaggacctggcccgaggattcttctggcaggtgctggaggccgtgcggcattgccacaactgcggggttctccaccgcgacatcaaggacgagaacatcttaatcgacctgagccgcggcgaaatcaaactcatcgacttcgggtcgggggcgctgctcaaggacacagtctacacggactttgatgggacccgagtgtacagtcctccagagtggattcgctaccatcgctaccacggcaggtcggcagctgtctggtcccttgggatcctgctctatgacatggtctgcggagatattccgtttgagcacgatgaagagatcatcaagggccaagtgttcttcaggcaaactgtctcttcagagtgtcagcaccttattaaatggtgcctgtccctgagaccatcagatcggccctcctttgaagaaatccggaaccatccatggatgcagggtgacctcctgccccaggcagcttctgagatccatctgcacagtctgtcaccggggtccagcaagtag

SEQ ID NO: 22 - DNA, 38 bp, Artificial
  hPIM1 CE oligo
gctcgggcctctccaggtttttctcttggaaagaaagt

SEQ ID NO: 23 - DNA, 42 bp, Artificial
  hPIM1 CE oligo
agtcgaagagatcttgcaccgtttttctcttggaaagaaagt

SEQ ID NO: 24 - DNA, 40 bp, Artificial
  hPIM1 CE oligo
ggccagctcctcttgcaggtttttctcttggaaagaaagt

SEQ ID NO: 25 - DNA, 38 bp, Artificial
  hPIM1 CE oligo
agctcgccgcgattgagtttttctcttggaaagaaagt

SEQ ID NO: 26 - DNA, 38 bp, Artificial
  hPIM1 CE oligo
ttgagcagcgcccccgatttttctcttggaaagaaagt

SEQ ID NO: 27 - DNA, 41 bp, Artificial
  hPIM1 CE oligo
catacagcaggatccccaggtttttctcttggaaagaaagt

SEQ ID NO: 28 - DNA, 46 bp, Artificial
  hPIM1 CE oligo
attctgaagagaccctctgcctgaatttttctcttggaaagaaagt

SEQ ID NO: 29 - DNA, 42 bp, Artificial
  hPIM1 CE oligo
gatttcttcgaaggttggccttttttctcttggaaagaaagt

SEQ ID NO: 30 - DNA, 43 bp, Artificial
  hPIM1 LE oligo
gcaggaccacttccatgggtttttaggcataggacccgtgtct

SEQ ID NO: 31 - DNA, 44 bp, Artificial
  hPIM1 LE oligo
acccgagctcaccttcttcatttttaggcataggacccgtgtct

SEQ ID NO: 32 - DNA, 43 bp, Artificial
  hPIM1 LE oligo
gcctaatgacgccggagaatttttaggcataggacccgtgtct

SEQ ID NO: 33 - DNA, 44 bp, Artificial
  hPIM1 LE oligo
cctctcgaaccagtccaggatttttaggcataggacccgtgtct

SEQ ID NO: 34 - DNA, 44 bp, Artificial
  hPIM1 LE oligo
atcaggacgaaactgtcgggtttttaggcataggacccgtgtct

SEQ ID NO: 35 - DNA, 43 bp, Artificial
  hPIM1 LE oligo
gctcccctttccgtgatgatttttaggcataggacccgtgtct

SEQ ID NO: 36 - DNA, 40 bp, Artificial
  hPIM1 LE oligo
gccgcacggcctccagtttttaggcataggacccgtgtct

SEQ ID NO: 37 - DNA, 42 bp, Artificial
  hPIM1 LE oligo
ccccgcagttgtggcagttttttaggcataggacccgtgtct

SEQ ID NO: 38 - DNA, 42 bp, Artificial
  hPIM1 LE oligo
tgatgtcgcggtgtagcatttttaggcataggacccgtgtct

SEQ ID NO: 39 - DNA, 47 bp, Artificial
  hPIM1 LE oligo
gtcgataaggatgttttcgtccttttttaggcataggacccgtgtct

SEQ ID NO: 40 - DNA, 45 bp, Artificial
  hPIM1 LE oligo
aagtccgtgtagacggtgtcctttttaggcataggacccgtgtct

SEQ ID NO: 41 - DNA, 45 bp, Artificial
  hPIM1 LE oligo
ctatacactcgggtcccatcgtttttaggcataggacccgtgtct

SEQ ID NO: 42 - DNA, 44 bp, Artificial
  hPIM1 LE oligo
ctgccatggtagcgatggtatttttaggcataggacccgtgtct

SEQ ID NO: 43 - DNA, 42 bp, Artificial
  hPIM1 LE oligo
gaccagactgccgccgactttttaggcataggacccgtgtct

SEQ ID NO: 44 - DNA, 48 bp, Artificial
  hPIM1 LE oligo
aaggaatatctccacacaccatattttttaggcataggacccgtgtct

SEQ ID NO: 45 - DNA, 48 bp, Artificial
  hPIM1 LE oligo
agcaccatctaatgagatgctgactttttaggcataggacccgtgtct

SEQ ID NO: 46 - DNA, 45 bp, Artificial
  hPIM1 LE oligo
ttgcatccatggatggttctgtttttaggcataggacccgtgtct

SEQ ID NO: 47 - DNA, 21 bp, Artificial
  hPIM1 BL oligo
cacctgccagaagaagctgcg

SEQ ID NO: 48 - DNA, 19 bp, Artificial
  hPIM1 BL oligo
cccgaagtcgatgagcttg

SEQ ID NO: 49 - DNA, 19 bp, Artificial
  hPIM1 BL oligo
gcggatccactctggaggg

SEQ ID NO: 50 - DNA, 20 bp, Artificial
  hPIM1 BL oligo
gatctcttcgtcatgctcga

SEQ ID NO: 51 - DNA, 18 bp, Artificial
  hPIM1 BL oligo
gaaaacctggcccctgat

SEQ ID NO: 52 - DNA, 20 bp, Artificial
  hPIM1 BL oligo
atctgatggtctcagggcca

SEQ ID NO: 53 - DNA, 43 bp, Artificial
  h cyclophilin CE oligo
atacaccttgacggtgactttgtttttctcttggaaagaaagt

SEQ ID NO: 54 - DNA, 44 bp, Artificial
  h cyclophilin CE oligo
cctttctctcctgtagctaaggctttttctcttggaaagaaagt

SEQ ID NO: 55 - DNA, 40 bp, Artificial
  h cyclophilin CE oligo
ggtgtctttgcctgcgttgtttttctcttggaaagaaagt

SEQ ID NO: 56 - DNA, 40 bp, Artificial
  h cyclophilin CE oligo
tgaagaactgggagccgtttttttctcttggaaagaaagt

SEQ ID NO: 57 - DNA, 41 bp, Artificial
  h cyclophilin CE oligo
gtctgtcttggtgctctccatttttctcttggaaagaaagt

SEQ ID NO: 58 - DNA, 40 bp, Artificial
  h cyclophilin CE oligo
tcaggggtttatcccggcttttttctcttggaaagaaagt

SEQ ID NO: 59 - DNA, 42 bp, Artificial
  h cyclophilin LE oligo
gccgcagaaggtcccggctttttaggcataggacccgtgtct

SEQ ID NO: 60 - DNA, 45 bp, Artificial
  h cyclophilin LE oligo
ggccccttcttcttctcatcgtttttaggcataggacccgtgtct

SEQ ID NO: 61 - DNA, 46 bp, Artificial
  h cyclophilin LE oligo
catctccaattcgtaggtcaaatttttaggcataggacccgtgtct

SEQ ID NO: 62 - DNA, 45 bp, Artificial
  h cyclophilin LE oligo
agatcacccggcctacatctttttttaggcataggacccgtgtct

SEQ ID NO: 63 - DNA, 48 bp, Artificial
  h cyclophilin LE oligo
cacaaaattatccactgtttttggtttttaggcataggacccgtgtct

SEQ ID NO: 64 - DNA, 47 bp, Artificial
  h cyclophilin LE oligo
atttgctgtttttgtagccaaattttttaggcataggacccgtgtct

SEQ ID NO: 65 - DNA, 43 bp, Artificial
  h cyclophilin LE oligo
agtctccgccctggatcattttttaggcataggacccgtgtct

SEQ ID NO: 66 - DNA, 44 bp, Artificial
  h cyclophilin LE oligo
tgtgccatctcccctggtgatttttaggcataggacccgtgtct

SEQ ID NO: 67 - DNA, 45 bp, Artificial
  h cyclophilin LE oligo
accgtagatgctctttcctcctttttaggcataggacccgtgtct

SEQ ID NO: 68 - DNA, 45 bp, Artificial
  h cyclophilin LE oligo
agttctcatcggggaagcgcttttttaggcataggacccgtgtct

SEQ ID NO: 69 - DNA, 47 bp, Artificial
  h cyclophilin LE oligo
aggcccgtagtgcttcagtttgatttttaggcataggacccgtgtct

SEQ ID NO: 70 - DNA, 41 bp, Artificial
  h cyclophilin LE oligo
gccatgctgacccagcctttttaggcataggacccgtgtct

SEQ ID NO: 71 - DNA, 44 bp, Artificial
  h cyclophilin LE oligo
acatgcttgccatctagccatttttaggcataggacccgtgtct

SEQ ID NO: 72 - DNA, 48 bp, Artificial
  h cyclophilin LE oligo
ccctctagaactttgccaaacacctttttaggcataggacccgtgtct

SEQ ID NO: 73 - DNA, 44 bp, Artificial
  h cyclophilin LE oligo
ccttccgcaccacctccatgtttttaggcataggacccgtgtct

SEQ ID NO: 74 - DNA, 46 bp, Artificial
  h cyclophilin LE oligo
cagtctgcgatgatcacatccttttttaggcataggacccgtgtct

SEQ ID NO: 75 - DNA, 42 bp, Artificial
  h cyclophilin LE oligo
tccacctcgatcttgccgtttttaggcataggacccgtgtct

SEQ ID NO: 76 - DNA, 42 bp, Artificial
  h cyclophilin LE oligo
gcgatggcaaagggcttctttttaggcataggacccgtgtct

SEQ ID NO: 77 - DNA, 23 bp, Artificial
  h cyclophilin BL oligo
aacagtctttccgaagagaccaa

SEQ ID NO: 78 - DNA, 23 bp, Artificial
  h cyclophilin BL oligo
gaagtccttgattacacgatgga

SEQ ID NO: 79 - DNA, 20 bp, Artificial
  h cyclophilin BL oligo
ggctgtcttgactgtcgtga

SEQ ID NO: 80 - protein, 801 aa, Artificial
  NGFR mammalian
MLRGXRXGQLGWHXXAAGXGXLXXXLXLAXAXAAXCXXXCCPXGXSGLRCTRXGXLXXLXXLXGAXNLTELYXENQXXLQXLEXXDLXGLGELRXLTIVKSGLRFVAPDAFXFTPRLSXLNLSXNALESLSWKTVQGLSLQXLXLSGNPLHCSCALXWLQRWEXEXLXGVXXQXLXXXGXGXXXXPLXHXXNXSCGVPXXKXQXPNXSVXVGDDVXLXCQVEGXXLXQAXWILTELEXXATXXKXGXLPSLGLXLXNVTSDLNXKNXTCWAENDVGRAEVSVQVXVSFPASVXLXXAVEXHHWCIPFSVDGQPAPSLRWXFNGSVLNETSFIFTXFLEXAXXNETXRHGCLRLNQPTHVNNGNYTLLAANPXGQAXASXMAAFMDNPFEFNPEDPIPVSFSPVDXNSTSXDPVEKKDETPFGVSVAVGLAVXAXLFLSXLLLVLNKCGXRXKFGINRPAVLAPEDGLAMSLHFMTLGGSSLSPTEGKGSGLQGHIXENPQYFSDXCVHHIKRXDIXLKWELGEGAFGKVFLAECXNLLXXQDKMLVAVKALKEXSEXARQDFXREAELLTMLQHQHIVRFFGVCTEGXPLLMVFEYMRHGDLNRFLRSHGPDAKLLAGGEDVAPGPLGLGQLLAVASQVAAGMVYLAXLHFVHRDLATRNCLVGQGLVVKIGDFGMSRDIYSTDYYRVGGRTMLPIRWMPPESILYRKFXTESDVWSFGVVLWEIFTYGKQPWYQLSNTEAIXCITQGRELERPRACPPXVYAIMRGCWQREPQQRXSXKDVHARLQALAQAPPXYLDVLG

SEQ ID NO: 81 - protein, 796 aa, Homo sapiens
MLRGGRRGQLGWHSWAAGPGSLLAWLILASAGAAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNLTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 82 - protein, 799 aa, Mus musculus
MLRGQRLGQLGWHRPAAGLGSLMTSLMLACASAASCREVCCPVGPSGLRCTRAGSLDTLRGLRGAGNLTELYVENQQHLQRLEFEDLQGLGELRSLTIVKSGLRFVAPDAFRFTPRLSHLNLSSNALESLSWKTVQGLSLQDLTLSGNPLHCSCALFWLQRWEQEGLCGVHTQTLHDSGPGDQFLPLGHNTSCGVPTVKIQMPNDSVEVGDDVFLQCQVEGLALQQADWILTELEGAATVKKFGDLPSLGLILVNVTSDLNKKNVTCWAENDVGRAEVSVQVSVSFPASVHLGLAVEQHHWCIPFSVDGQPAPSLRWLFNGSVLNETSFIFTQFLESALTNETMRHGCLRLNQPTHVNNGNYTLLAANPYGQAAASVMAAFMDNPFEFNPEDPIPVSFSPVDGNSTSRDPVEKKDETPFGVSVAVGLAVSAALFLSALLLVLNKCGQRSKFGINRPAVLAPEDGLAMSLHFMTLGGSSLSPTEGKGSGLQGHIMENPQYFSDTCVHHIKRQDIILKWELGEGAFGKVFLAECYNLLNDQDKMLVAVKALKEASENARQDFQREAELLTMLQHQHIVRFFGVCTEGGPLLMVFEYMRHGDLNRFLRSHGPDAKLLAGGEDVAPGPLGLGQLLAVASQVAAGMVYLASLHFVHRDLATRNCLVGQGLVVKIGDFGMSRDIYSTDYYRVGGRTMLPIRWMPPESILYRKFSTESDVWSFGVVLWEIFTYGKQPWYQLSNTEAIECITQGRELERPRACPPDVYAIMRGCWQREPQQRLSMKDVHARLQALAQAPPSYLDVLG

SEQ ID NO: 83 - protein, 799 aa, Rattus norvegicus
MLRGQRHGQLGWHRPAAGLGGLVTSLMLACACAASCRETCCPVGPSGLRCTRAGTLNTLRGLRGAGNLTELYVENQRDLQRLEFEDLQGLGELRSLTIVKSGLRFVAPDAFHFTPRLSHLNLSSNALESLSWKTVQGLSLQDLTLSGNPLHCSCALLWLQRWEQEDLCGVYTQKLQGSGSGDQFLPLGHNNSCGVPSVKIQMPNDSVEVGDDVFLQCQVEGQALQQADWILTELEGTATMKKSGDLPSLGLTLVNVTSDLNKKNVTCWAENDVGRAEVSVQVSVSFPASVHLGKAVEQHHWCIPFSVDGQPAPSLRWFFNGSVLNETSFIFTQFLESALTNETMRHGCLRLNQPTHVNNGNYTLLAANPYGQAAASIMAAFMDNPFEFNPEDPIPVSFSPVDTNSTSRDPVEKKDETPFGVSVAVGLAVSAALFLSALLLVLNKCGQRSKFGINRPAVLAPEDGLAMSLHFMTLGGSSLSPTEGKGSGLQGHIMENPQYFSDTCVHHIKRQDIILKWELGEGAFGKVFLAECYNLLNDQDKMLVAVKALKETSENARQDFHREAELLTMLQHQHIVRFFGVCTEGGPLLMVFEYMRHGDLNRFLRSHGPDAKLLAGGEDVAPGPLGLGQLLAVASQVAAGMVYLASLHFVHRDLATRNCLVGQGLVVKIGDFGMSRDIYSTDYYRVGGRTMLPIRWMPPESILYRKFSTESDVWSFGVVLWEIFTYGKQPWYQLSNTEAIECITQGRELERPRACPPDVYAIMRGCWQREPQQRLSMKDVHARLQALAQAPPSYLDVLG

SEQ ID NO: 84 - protein, 241 aa, Artificial
  NGF mammalian
MSMLFYTLITXXLIGXQAEXXXXSNXXXGXXXPXXHWTKLQHSLDTALRRAXSXPXXXIAARVXGQTXNITVDPXLFKKRXLXSPRVLFSTQPPXXXXDXXDLDFXXXGXXXXNRTHRSKRSSXHPXFXXGEFSVCDSVSVWVXDKTTATDIKGXEVXVLXEVNXNNXVFXQYFFETKCRXXXPVXSGCRGIDSKHWNSYCTTTHTFVKALTXXXXQAAWRFIRIDTACVCVLXRKAXRRX

SEQ ID NO: 85 - protein, 241 aa, Homo sapiens
MSMLFYTLITAFLIGIQAEPHSESNVPAGHTIPQVHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 86 - protein, 241 aa, Gorilla gorilla
MSMLFYTLITAFLIGIQAELHSESNVPAGHTIPQAHWTKLQHSLDTALRRARSAPAAAIAARVAGQTRNITVDPRLFKKRRLRSPRVLFSTQPPPEAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 87 - protein, 241 aa, Pongo pygmaeus
MSMLFYTLITAFLIGIQAEPHSESNVPAGHTIPQAHWTKLQHSLDTALRRARSTPAAAIAARVAGQTCNITVDPRLFKKRRLRSPRVLFSTQPPPEAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 88 - protein, 241 aa, Rattus norvegicus
MSMLFYTLITAFLIGVQAEPYTDSNVPEGDSVPEAHWTKLQHSLDTALRRARSAPAEPIAARVTGQTRNITVDPKLFKKRRLRSPRVLFSTQPPPTSSDTLDLDFQAHGTISFNRTHRSKRSSTHPVFHMGEFSVCDSVSVWVGDKTTATDIKGKEVTVLGEVNINNSVFKQYFFETKCRAPNPVESGCRGIDSKHWNSYCTTTHTFVKALTTDDKQAAWRFIRIDTACVCVLSRKAARRG

SEQ ID NO: 89 - protein, 241 aa, Mus musculus
MSMLFYTLITAFLIGVQAEPYTDSNVPEGDSVPEAHWTKLQHSLDTALRRARSAPTAPIAARVTGQTRNITVDPRLFKKRRLHSPRVLFSTQPPPTSSDTLDLDFQAHGTIPFNRTHRSKRSSTHPVFHMGEFSVCDSVSVWVGDKTTATDIKGKEVTVLAEVNINNSVFRQYFFETKCRASNPVESGCRGIDSKHWNSYCTTTHTFVKALTTDEKQAAWRFIRIDTACVCVLSRKATRRG

SEQ ID NO: 90 - protein, 241 aa, Cavia porcellus
MSMLFYTLITVFLIGIQAEPYSDSNVLSGDTIPQAHWTKLQHSLDTALRRAHSAPAAPIAARVAGQTLNITVDPRLFKKRRLHSPRVLFSTQPPPLSTDAQDLDFEVDGAASVNRTHRSKRSSTHPVFHMGEFSVCDSVSVWVADKTTATDIKGKEVTVLAEVNVNNNVFKQYFFETKCRDPSPVDSGCRGIDSKHWNSYCTTTHTFVKALTTANKQAAWRFIRIDTACVCVLNRKAARRG

SEQ ID NO: 91 - protein, 241 aa, Mastomys natalensis
MSMLFYTLITALLIGVQAEPYTDSNLPEGDSVPEAHWTKLQHSLDTALRRARSAPAAPIAARVTGQTRNITVDPRLFKKRKLRSPRVLFSTQPPPTSSDTLDLDFQAHGTISFNRTHRSKRSSTHPVFQMGEFSVCDSVSVWVGDKTTATDIKGNEVTVLGEVNINNSVFKQYFFETKCRARNPVESGCRGIDSKHWNSYCTTTHTFVKALTTDDRQAAWRFIRIDTACVCVLTRKAPRRG

SEQ ID NO: 92 - protein, 189 aa, Artificial
  BAFFR mammalian
XXXXRXXXRSXRXRDXXXPTXCXXXECFDXLVRXCVXCXLXXTXXPXXXXXSSXXPXTALQPQEXXXXXXGXAXXPXXXLLXGAPALLGLXLXLXLVXLVXLVSWRXRQXXLRXASXXXXPDXXXXXXXEXLXXVXXXSXXXXXAXAPXWPPXXEDXXXXXPXHSVPVPATELGSTELVTTKTAGPEQX

SEQ ID NO: 93 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 94 - protein, 175 aa, Mus musculus
MGARRLRVRSQRSRDSSVPTQCNQTECFDPLVRNCVSCELFHTPDTGHTSSLEPGTALQPQEGSALRPDVALLVGAPALLGLILALTLVGLVSLVSWRWRQQLRTASPDTSEGVQQESLENVFVPSSETPHASAPTWPPLKEDADSALPRHSVPVPATELGSTELVTTKTAGPEQ

SEQ ID NO: 95 - protein, 318 aa, Artificial
  BAFFR mammalian
MDXSXXXXXXXXLXXCXXKXEXMKXXXXXXIXPXKEXXXXXXXXXDGXLLAATLLLALLSXXXTXXSXYQXAALQXDLXXLRXELQXXXXXXXPAXAGAPXXXXXXXXXXTAGXKXXXPXAPXXXNSSXXXRNXRAXQGPEETXXXXXXXXXXXXXXXXXXXXXXXXXXXXXXXXXQDCLQLIADSXTPTIXKGXYTFVPWLLSFKRGXALEEKENKIXVXXTGYFFIYXQVLYTDXXXAMGHXIQRKKVHVFGDELSLVTLFRCIQNMPXTLPNNSCYSAGIAXLEEGDEXQLAIPRENAQISXXGDXTFFGALKLL

SEQ ID NO: 96 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 97 - protein, 309 aa, Mus musculus
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 98 - protein, 469 aa, Artificial
  TNFR1 mammalian
MGLXXVPXLLXXLVLXXLLXXXXPXXVXGLVXXXXDREKRXXXCPQGKYXHXXXXSICCTKCHKGTYLXXDCXXPGXXTXCXXCXXGXFTASXNXXXXCLSCXXCRXEMXQVEISXCXXXXDTVCGCXXNQXXXYXSEXXFQCXXCSXCXNGTVXXXCXEXQXTXCXCHXGFFLXXXECXXCXXCXKXXXCXXLCXXXXXXXXXXXXDXGTXVLLPLVIXXGXCXXXXXXXXLXXRYXXXXXXXXSIXCXXXXPXKEXEXXXXXXXPXXXXXXXXFSPXXXXXPTXXFSXXPXXXXXXSXTXXXXXXXPXXXXXXXXXXXXXEXXPXXQGAXPXLXXXXXXXPXXXXXXKWXXXXXXXXXXPXXXXXXDXAXLYAVVXXVPPXRWKEFXRXXGLSXHEIXRLEXQNGRCLREAXYSMLXXWRRRTXRXEXTLXXXGXVLXXMXLXGCLEXIXEXLXXPAXXXXXXXLXR

SEQ ID NO: 99 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 100 - protein, 446 aa, Felis catus
MGLPTVPGLLQPLVLLALLVEIYPLRVTGLVPHLRDREKRAIPCPQGKYIHPQDNSICCTKCHKGTYLYNDCAGPGLDTDCRECENGTFTASENYLRQCLSCSKCRKEMYQVEISPCTVYRDTVCGCRKNQYRYYWSETHFQCLNCSLCLNGTVQISCKETQNTVCTCHAGFFLRGNECVSCVNCKKNTECTKLCVPIVETVKDPQDPGTTVLLPLVIFFGICVLSFSIGLMCRYQRRKSKLFSIVCGKSTPTKEGEPQPLATGPGFSPIPSPTFSPSPTFTPSPTFTPSDWANLRAASVSREMAPPYQGAGPILSAAPASSPISTPVQKWEDSTHTQRPEADPADPATLYAVVDGVPPSRWKEFVRRLGLSEHEIERLELQNGRCLREAHYSMLAAWRRRTPRREATLELLGRVLRDMDLLGCLEDIEEALCAPASLSPAPRLLR

SEQ ID NO: 101 - protein, 461 aa, Sus scrofa
MGLSTVPGLLLPLVLRALLVDVYPAGVHGLVLHPGDREKRESLCPQGKYSHPQNRSICCTKCHKGTYLHNDCLGPGLDTDCRECDNGTFTASENHLTQCLSCSKCRSEMSQVEISPCTVDRDTVCGCRKNQYRKYWSETLFQCLNCSLCPNGTVQLPCLEKQDTICNCHSGFFLRDKECVSCVNCKNADCKNLCPATSETRNDFQDTGTTVLLPLVIFFGLCLAFFLFVGLACRYQRWKPKLYSIICGKSTPVKEGEPEPLATAPSFGPITTFSPIPSFSPTTTFSPVPSFSPISSPTFTPCDWSNIKVTSPPKEIAPPPQGAGPILPMPPASTPVPTPLPKWGGSAHSAHSAPAQLADADPATLYAVVDGVPPTRWKEFVRRLGLSEHEIERLELQNGRCLREAQYSMLAEWRRRTSRREATLELLGSVLRDMDLLGCLEDIEEALRGPARLAPAPHLLR

SEQ ID NO: 102 - protein, 461 aa, Rattus norvegicus
MGLPIVPGLLLSLVLLALLMGIHPSGVTGLVPSLGDREKRDNLCPQGKYAHPKNNSICCTKCHKGTYLVSDCPSPGQETVCEVCDKGTFTASQNHVRQCLSCKTCRKEMFQVEISPCKADMDTVCGCKKNQFQRYLSETHFQCVDCSPCFNGTVTIPCKEKQNTVCNCHAGFFLSGNECTPCSHCKKNQECMKLCLPPVANVTNPQDSGTAVLLPLVIFLGLCLLFFICISLLCRYPQWRPRVYSIICRDSAPVKEVEGEGIVTKPLTPASIPAFSPNPGFNPTLGFSTTPRFSHPVSSTPISPVFGPSNWHNFVPPVREVVPTQGADPLLYGSLNPVPIPAPVRKWEDVVAAQPQRLDTADPAMLYAVVDGVPPTRWKEFMRLLGLSEHEIERLELQNGRCLREAHYSMLEAWRRRTPRHEATLDVVGRVLCDMNLRGCLENIRETLESPAHSSTTHLPR

SEQ ID NO: 103 - protein, 454 aa, Mus musculus
MGLPTVPGLLLSLVLLALLMGIHPSGVTGLVPSLGDREKRDSLCPQGKYVHSKNNSICCTKCHKGTYLVSDCPSPGRDTVCRECEKGTFTASQNYLRQCLSCKTCRKEMSQVEISPCQADKDTVCGCKENQFQRYLSETHFQCVDCSPCFNGTVTIPCKETQNTVCNCHAGFFLRESECVPCSHCKKNEECMKLCLPPPLANVTNPQDSGTAVLLPLVILLGLCLLSFIFISLMCRYPRWRPEVYSIICRDPVPVKEEKAGKPLTPAPSPAFSPTSGFNPTLGFSTPGFSSPVSSTPISPIFGPSNWHFMPPVSEVVPTQGADPLLYESLCSVPAPTSVQKWEDSAHPQRPDNADLAILYAVVDGVPPARWKEFMRFMGLSEHEIERLEMQNGRCLREAQYSMLEAWRRRTPRHEDTLEVVGLVLSKMNLAGCLENILEALRNPAPSSTTRLPR

SEQ ID NO: 104 - DNA, 300 bp, Homo sapiens
atgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataa

SEQ ID NO: 105 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 106 - protein, 316 aa, Mus musculus
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENAGLQDSTLESEDTLPDSCRRMKQAFQGAVQKELQHIVGPQRFSGAPAMMEGSWLDVAQRGKPEAQPFAHLTINAASIPSGSHKVTLSSWYHDRGWAKISNMTLSNGKLRVNQDGFYYLYANICFRHHETSGSVPTDYLQLMVYVVKTSIKIPSSHNLMKGGSTKNWSGNSEFHFYSINVGGFFKLRAGEEISIQVSNPSLLDPDQDATYFGAFKVQDID

SEQ ID NO: 107 - protein, 318 aa, Rattus norvegicus
MRRANRDYGKYLRGSEEMGSCPGVPHEGPLHPAPSAPAPAPPPAASRFMFLALLGLGLGQVVCSIALFLYFRAQMDPNRISEDSTRCFYRILRLRENTGLQDSTLESEDTEALPDSCRRMKQAFQGAVQRELQHIVGPQRFSGVPAMMEGSWLDVARRGKPEAQPFAHLTINAADIPSGSHKVSLSSWYHDRGWAKISNMTLSNGKLRVNQDGFYYLYANICFRHHETSGSVPADYLQLMVYVVKTSIKIPSSHNLMKGGSTKNWSGNSEFHFYSINVGGFFKLRAGEEISVQVSNPSLLDPDQDATYFGAFKVQDID

SEQ ID NO: 108 - protein, 616 aa, Homo sapiens
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 109 - protein, 625 aa, Mus musculus
MAPRARRRRQLPAPLLALCVLLVPLQVTLQVTPPCTQERHYEHLGRCCSRCEPGKYLSSKCTPTSDSVCLPCGPDEYLDTWNEEDKCLLHKVCDAGKALVAVDPGNHTAPRRCACTAGYHWNSDCECCRRNTECAPGFGAQHPLQLNKDTVCTPCLLGFFSDVFSSTDKCKPWTNCTLLGKLEAHQGTTESDVVCSSSMTLRRPPKEAQAYLPSLIVLLLFISVVVVAAIIFGVYYRKGGKALTANLWNWVNDACSSLSGNKESSGDRCAGSHSATSSQQEVCEGILLMTREEKMVPEDGAGVCGPVCAAGGPWAEVRDSRTFTLVSEVETQGDLSRKIPTEDEYTDRPSQPSTGSLLLIQQGSKSIPPFQEPLEVGENDSLSQCFTGTESTVDSEGCDFTEPPSRTDSMPVSPEKHLTKEIEGDSCLPWVVSSNSTDGYTGSGNTPGEDHEPFPGSLKCGPLPQCAYSMGFPSEAAASMAEAGVRPQDRADERGASGSGSSPSDQPPASGNVTGNSNSTFISSGQVMNFKGDIIVVYVSQTSQEGPGSAEPESEPVGRPVQEETLAHRDSFAGTAPRFPDVCATGAGLQEQGAPRQKDGTSRPVQEQGGAQTSLHTQGSGQCAE

(19) United States
(12) Patent Application Publication
(10) Pub. No.: US 2006/0211022 A1
(43) Pub. Date: Sep. 21, 2006

(54) Detection of compounds that affect therapeutic activity

(75) Inventors: Shuqian Jing, Palo Alto, CA (US); Francesca Civoli, Newbury Park, CA (US); Shalini Gupta, Newbury Park, CA (US); Daniel Steven Halperin, Calabasas, CA (US); Jason Joseph Pennucci, Santa Monica, CA (US); Steven Swanson, Moorpark, CA (US); Yan Bin Yu, Thousand Oaks, CA (US)

Correspondence Address: AMGEN INC., 1120 VETERANS BOULEVARD, SOUTH SAN FRANCISCO, CA, 94080, US

(73) Assignee: Amgen Inc., Thousand Oaks, CA (US)

(21) Appl. No.: 11/361,415

(22) Filed: Feb. 23, 2006

Related U.S. Application Data
(60) Provisional application No. 60/656,696, filed on Feb. 24, 2005.

(51) Int. Cl. C12Q 1/68; G01N 33/53

(52) U.S. Cl. 435/6; 435/7.1

(57) ABSTRACT

The present invention relates to methods of detecting compounds that affect the activity of a therapeutic substance or composition administered to a subject, and to reagents for use in such methods.

### BACKGROUND OF THE INVENTION

Subjects being treated with therapeutic substances or compositions may experience changes in the activity or the effectiveness of the therapeutic substance because of the presence of certain compounds in the subject. For example, the administration of a therapeutic substance may result in the formation of antibodies against that therapeutic substance by the subject to whom the therapeutic substance was administered. In particular, if such anti-therapeutic antibodies are neutralizing antibodies that prevent the beneficial activity of the therapeutic substance, this phenomenon can have an adverse effect on the treatment of the subject.

Cytokines or growth factors exert their biologic effects by binding to their receptors and activating various intracellular signal transduction processes (Schlessinger and Ullrich (1992) Neuron 9: 383-391; Kishimoto et al. (1994) Cell 76: 253-262; Ihle (1995) Nature 377: 591-594; Wells (1996), Proc. Natl. Acad. Sci. USA 93: 1-6; Dhanasekaran (1998), Oncogene 17: 1329-1330). The synergistic action of the activated intracellular signaling pathways causes alterations in gene expression and further leads to changes in cell survival, proliferation or apoptosis (Kishimoto et al. (1994); Ihle (1995); Appleby et al., (1996) Cell 86: 845-848; Dhanasekaran 1998). These changes reflecting the biologic effects of the growth factors or cytokines have been widely used as biomarkers in existing cell-based bioassays for determining the quantities of biologically active cytokines or growth factors (Mire-Sluis (2001) Pharm. Research 18: 1239-46; Eghbali-Fatourechi et al. (1996) Endocrinology 137(5): 1894-903). In the biomedical field, these types of assays are used to detect and characterize serum neutralizing antibodies against therapeutics, particularly protein therapeutics of which many are growth factors or cytokines.

The most widely used bioassay for serum neutralizing antibodies assesses cell proliferation by measuring the uptake of a radioisotope-labeled nucleotide, [³H]-thymidine (Eghbali-Fatourechi et al. (1996); Mire-Sluis (2001)). This approach can be used as long as the cells respond to the therapeutic agent by proliferating. By monitoring the amount of [³H]-Thymidine incorporated into chromosomes, either induction or inhibition of cell proliferation can be measured. When a neutralizing antibody is present, the therapeutic agent-induced proliferation is blocked. The major advantage of this method is its reliability and high sensitivity. The use of radioactive materials makes the method potentially hazardous and the disposal of radioactive waste increases the experimental costs, however. In addition, using cell proliferation as the final readouts results in a long assay duration time, typically ranging from 3 to 5 days.

Therefore, there is a need for reagents and safe, sensitive and effective methods for the detection of compounds that affect the activity of therapeutic substances and compositions.

### SUMMARY OF THE INVENTION

The present invention provides methods for detecting the presence of a compound in a sample, comprising the following steps: providing, in any order: a sample suspected of comprising a compound and a control sample without the compound; a receptor and a response gene; and a ligand, wherein the ligand is capable of binding the receptor, thereby altering the expression of the response gene; combining, in any order, (i) the sample, the receptor, and the ligand; and (ii) the control sample, the receptor and the ligand; and measuring the level of the expression of the response gene; wherein the presence of the compound in the sample is detected by an alteration in the level of expression of the response gene when compared to the level of expression of the response gene when the receptor is combined with the ligand in the presence of the control sample. In one aspect, the invention provides methods for measuring the amount of a compound in a sample.

The invention further provides methods for detecting the presence of a compound in the presence or absence of a sample, comprising: providing, in any order: a compound, wherein the compound is in the presence or absence of a sample; a receptor and a response gene; and a ligand, wherein the ligand is capable of binding the receptor, thereby altering the expression of the response gene; combining, in any order, (i) the compound, the receptor, and the ligand; and (ii) the receptor and the ligand; and measuring the level of the expression of the response gene, wherein the presence of the compound is measured by an alteration in the level of expression of the response gene when the receptor is combined with the ligand and the compound compared to the level of expression of the response gene when the receptor is combined with the ligand only; and wherein when the receptor is combined with varying concentrations of the ligand and the compound, the expression of the response gene in the presence of the sample is correlated with the expression of the response gene in the absence of the sample with a correlation coefficient of at least 0.5. In one aspect, the method can be used for measuring the amount of the compound in the presence or absence of the sample.

In one aspect, the ligand can be a therapeutic substance for administration to a subject. In one aspect, the compound can be a neutralizing antibody against the therapeutic substance.

In one aspect, the receptor comprises SEQ ID NO:1. In another aspect, the receptor can comprise SEQ ID NO:2, SEQ ID NO:3, SEQ ID NO:4, or SEQ ID NO:5.

In one aspect, the therapeutic substance comprises SEQ ID NO:6. In another aspect, the therapeutic substance can comprise SEQ ID NO:7, SEQ ID NO:8, SEQ ID NO:9, SEQ ID NO:10, SEQ ID NO:11, SEQ ID NO:12, SEQ ID NO:13, or SEQ ID NO:14. In one aspect, the response gene can comprise SEQ ID NO:15. In another aspect, the response gene can comprise SEQ ID NO:16, SEQ ID NO:17, SEQ ID NO:18, SEQ ID NO:19, SEQ ID NO:20, or SEQ ID NO:21.

In one aspect, the receptor can comprise the extracellular domain of SEQ ID NO:80. In another aspect, the receptor can comprise the extracellular domain of SEQ ID NO:81, SEQ ID NO:82, or SEQ ID NO:83. In one aspect, the ligand can comprise SEQ ID NO:84. In another aspect, the ligand can comprise SEQ ID NO:85, SEQ ID NO:86, SEQ ID NO:87, SEQ ID NO:88, SEQ ID NO:89, SEQ ID NO:90, or SEQ ID NO:91. In one aspect, the response gene can comprise SEQ ID NO:15.

In one aspect, the receptor can comprise the extracellular domain of SEQ ID NO:92. In another aspect, the receptor can comprise the extracellular domain of SEQ ID NO:93 or SEQ ID NO:94. In one aspect, the ligand can comprise SEQ ID NO:95. In another aspect, the ligand can comprise SEQ ID NO:96 or SEQ ID NO:97. In one aspect, the response gene can comprise SEQ ID NO:98. In another aspect, the response gene can comprise SEQ ID NO:99, SEQ ID NO:100, SEQ ID NO:101, SEQ ID NO:102, or SEQ ID NO:103.

In one aspect, the response gene can comprise SEQ ID NO:15.

In one aspect, the ligand can comprise SEQ ID NO:105, SEQ ID NO:106, or SEQ ID NO:107. In one aspect, the receptor can comprise SEQ ID NO:108 or SEQ ID NO:109. In one aspect, the response gene is tartrate resistant acid phosphatase (TRAP).

In one aspect, the invention provides methods for detecting the presence of a compound in a sample or measuring the amount of a compound in a sample, wherein the ligand is an endogenous ligand, which is bound by a therapeutic substance for administration to a subject.

In one aspect, the level of the expression of the response gene is measured using a branched DNA (bDNA) assay.

In one aspect, the sample can be selected from the group consisting of whole blood, plasma, serum, synovial fluid, ascitic fluid, lacrimal fluid, perspiration, seminal fluid, cell extracts, and tissue extracts.

In one aspect, the invention provides methods for detecting the presence of a compound in a sample or measuring the amount of a compound in a sample, wherein the receptor is expressed by a mammalian cell.

In one aspect, the invention provides a kit comprising (a) a cell expressing a receptor, wherein the receptor comprises the intracellular domain of EPOR, and (b) one or more oligonucleotides used to detect PIM1 gene expression, the oligonucleotides selected from the group consisting of SEQ ID NOs: 22 through 79.

### BRIEF DESCRIPTION OF THE DRAWINGS

FIG. 1 illustrates the EPO-induced PIM-1 expression in UT-7 cells. The level of PIM-1 mRNA expression in UT-7 cells treated with human rSCF, rG-CSF, rEPO, rGM-CSF, and mouse rIL-3 at indicated concentrations (A), or with 2 ng/mL of rEPO for indicated periods of times (B) was determined by using bDNA technology and compared with that in the untreated cells.

FIG. 2 schematically represents inhibition of EPO-induced PIM-1 expression by PI3-K antagonist. UT-7 cells pretreated with inhibitors for PI3-K, MAPK, PKA, and PKC and un-pretreated cells were treated with (+Epo) or without (−Epo) rEPO for 90 minutes (A) or 24 (B) hours. The levels of PIM-1 expression in cells treated with rEPO for 90 minutes were compared with that in untreated cells (A). The numbers of cells in cultures treated with or without rEPO for 24 hours were determined (B).

FIG. 3 illustrates EPO-induced PIM-1 expression in UT-7 cells in the presence of normal human serum. UT-7 cells were treated with 3 ng/mL of rEPO for 90 minutes in the presence or absence of indicated concentrations of normal human serum (A) or with rEPO at indicated concentrations for 90 minutes in the presence or absence of 10% normal human serum (B). The level of PIM-1 mRNA in each sample was determined and compared with that in untreated cells.

FIG. 4 is a schematic representation of inhibition of EPO-induced PIM-1 expression by Anti-EPO neutralizing antibody. UT-7 cells were treated with 0.6 ng/mL of rEPO with indicated concentrations of the anti-Epo neutralizing antibody in the presence or absence of 10% normal human serum. The expression level of PIM-1 in each sample was compared with that of untreated control cells.

FIG. 5 illustrates the detection of anti-EPO neutralizing antibodies in serum. UT-7 cells were treated with 10% of pooled (PNHS) or individual (D1-D10) normal human donor serum spiked with 200 or 400 ng/mL anti-EPO neutralizing antibody 29123 in the presence (purple, yellow, and orange bars) or absence (blue bars) of 0.6 ng/ml of rEpo at 37oC for 1.5 hours. The expression level of PIM-1 in each sample was determined by using bDNA technology. The cutoff line for assigning the presence of an anti-EPO neutralizing antibody (Emax/2) is calculated by (PIM-1 expression in cells treated with 10% pHS only + PIM-1 expression in cells treated with 10% pHS containing 0.6 ng/mL of rEPO)/2.

FIG. 6 schematically represents a comparison of gene expression and [3H]-Thymidine incorporation assay platforms. The levels of PIM-1 expression in UT-7 cells treated with indicated concentrations of rEPO (FIG. 6A), or with 0.6 ng/mL of rEPO and indicated concentrations of the anti-EPO antibody (FIG. 6B) for 1.5 hours were determined and compared with that in untreated control cells.

FIG. 7 is a schematic representation of NGF and EPO hybrid receptors.

FIG. 8 represents the generic cloning strategy for making NGFR/EPOR hybrid receptors.

FIG. 9 is a schematic representation of BAFFR and TNFR1 hybrid receptors.

FIG. 10 represents the generic cloning strategy for making BAFFR/TNFR1 hybrid receptors.

FIG. 11 is a schematic representation of IL-8 production (pg/ml) induced by BAFF in COS-1 cells transfected with BAFF/TNFR constructs.

FIG. 12 is a schematic representation of BAFFR and EPO hybrid receptors.

FIG. 13 represents the generic cloning strategy for making BAFFR/EPOR hybrid receptors.

FIG. 14 is a schematic representation of BAFF-induced PIM-1 expression in 32D cells expressing BMCEB constructs.

FIG. 15 is a schematic representation of the Nab Assay for RANK/RANK ligand in 29 human donors FIG. 16 represents the validation of the Specificity Value Threshold for RANK assay.

### DETAILED DESCRIPTION OF THE INVENTION

I. Summary

The invention is directed to methods of detecting compounds that affect the activity of therapeutic substances or compositions, and to materials to be used in such methods. In one aspect, the method of the invention determines the activity of a therapeutic substance by measuring a cellular response to the binding between the therapeutic substance and a receptor for that substance, and comparing that response to the level of the response in the presence of a compound or compounds that may affect the binding between the therapeutic substance and its receptor. In other aspects, for example in cases where the therapeutic substance binds to an endogenous ligand and prevents or decreases the binding between the endogenous ligand and its receptor, the cellular response to binding of the receptor to its endogenous ligand is measured in the presence and absence of a compound that may affect the interaction of the therapeutic substance with the endogenous ligand.

II. Definitions “Polypeptide” is defined herein as natural, synthetic, and recombinant proteins or peptides generally having more than 10 amino acids. A “polypeptide linker” can be a polypeptide formed by a series of amino acids as short as one amino acid in length.

“Isolated”, as used herein, refers to a polypeptide or other molecule that has been removed from the environment in which it naturally occurs.

“Substantially purified”, as used herein, refers to a polypeptide that is substantially free of other polypeptides present in the environment in which it naturally occurs or in which it was produced; a preparation of a polypeptide that has been substantially purified contains at least 90% by weight (or at least 95%, at least 98%, or at least 99% by weight) of that polypeptide, wherein the weight of the polypeptide includes any carbohydrate, lipid, or other residues covalently attached to the polypeptide. A substantially purified polypeptide preparation may contain variation among polypeptide molecules within the preparation, with respect to extent and type of glycosylation or other post-translation modification, or with respect to conformation or extent of multimerization.

“Purified polypeptide”, as used herein, refers to an essentially homogenous polypeptide preparation; however, an essentially homogenous polypeptide preparation may contain variation among polypeptide molecules within the preparation, with respect to extent and type of glycosylation or other post-translation modification, or with respect to conformation or extent of multimerization.

“Full-length” polypeptides are those having the complete primary amino acid sequence of the polypeptide as initially translated; for example, the full-length form of the human EPO-R is shown as SEQ ID NO:2. The “mature form” of a polypeptide refers to a polypeptide that has undergone post-translational processing steps such as cleavage of the signal sequence and/or by proteolytic cleavage to remove a prodomain. Multiple mature forms of a particular full-length polypeptide may be produced, for example by cleavage of the signal sequence at multiple sites, or by differential regulation of proteases that cleave the polypeptide. The mature form(s) of such polypeptide can be obtained by expression, in a suitable mammalian cell or other host cell, of a nucleic acid molecule that encodes the full-length polypeptide. The sequence of the mature form of the polypeptide may also be determinable from the amino acid sequence of the full-length form, through identification of signal sequences or protease cleavage sites. In certain aspects, the mature form of the human EPO-R polypeptide has amino acid positions within the corresponding SEQ ID NOs as represented in Table 6.

The “percent identity” of two amino sequences can be determined by visual inspection and mathematical calculation, and the comparison can also be done by comparing sequence information using a computer program. The first step in determining percent identity is aligning the amino acid sequences to so as to maximize overlap and identities, while minimizing gaps in the alignment. The second step in determining percent identity is calculation of the number of identities between the aligned sequences, divided by the total number of amino acids in the alignment. When determining the percent identity that an amino acid sequence has “across the length of” a target amino acid sequence, the length of the target amino acid sequence is the minimum value for the number of total bases in the alignment. For example, when determining the percent identity of a first amino acid sequence of 50 amino acids “across the length of” a second amino acid sequence of amino acids 1 through 100 of SEQ ID NO:X, if the first amino acid sequence is identical to amino acids 1 through 50 of SEQ ID NO:X, the percent identity would be 50%: 50 amino acid identities divided by the total length of the alignment (100 amino acids). An exemplary computer program for aligning amino acid sequences and computing percent identity is the BLASTP program available for use via the National Library of Medicine website ncbi.nlm.nih.gov/gorf/wblast2.cgi, or the UW-BLAST 2.0 algorithm. Standard default parameter settings for UW-BLAST 2.0 are described at the following Internet site: sapiens.wustl.edu/blast/blast/README.html. In addition, the BLAST algorithm uses the BLOSUM62 amino acid scoring matrix, and optional parameters that can be used are as follows: (A) inclusion of a filter to mask segments of the query sequence that have low compositional complexity (as determined by the SEG program of Wootton and Federhen (Computers and Chemistry, 1993); also see Wootton and Federhen, 1996, Analysis of compositionally biased regions in sequence databases, Methods Enzymol. 266: 554-71) or segments consisting of short-periodicity internal repeats (as determined by the XNU program of Clayerie and States (Computers and Chemistry, 1993)), and (B) a statistical significance threshold for reporting matches against database sequences, or E-score (the expected probability of matches being found merely by chance, according to the stochastic model of Karlin and Altschul (1990); if the statistical significance ascribed to a match is greater than this E-score threshold, the match will not be reported.); E-score threshold values are 0.5, 0.25, 0.1, 0.05, 0.01, 0.001, 0.0001, 1e-5, 1e-10, 1e-15, 1e-20, 1e-25, 1e-30, 1e-40, 1e-50, 1e-75, or 1e-100. Other programs used by those skilled in the art of sequence comparison can also be used to align amino acid sequences, such as, the Genetics Computer Group (GCG; Madison, Wis.) Wisconsin package version 10.0 program, ‘GAP’ (Devereux et al., 1984, Nucl. Acids Res. 12: 387). The default parameters for the ‘GAP’ program include: (1) The GCG implementation of a unary comparison matrix (containing a value of 1 for identities and 0 for non-identities) for nucleotides, and the weighted amino acid comparison matrix of Gribskov and Burgess, Nucl. Acids Res. 14:6745, 1986, as described by Schwartz and Dayhoff, eds., Atlas of Polypeptide Sequence and Structure, National Biomedical Research Foundation, pp. 353-358, 1979; or other comparable comparison matrices; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps.

“Hybrid receptor” generally comprises an intracellular domain of one polypeptide joined to an extracellular domain of another polypeptide. The hybrid receptor further comprises a trans-membrane domain, which may be derived from either receptor or comprise a portion of one receptor and a portion of another one.

In one aspect, the extracellular domain includes amino acids 1-416, 1-417, 1-419, 1-422, or 1-424 of SEQ ID NO:81. In one aspect, the intracellular domain includes amino acid residues 274-507 of SEQ ID NO:3 In one aspect, the trans-membrane domain includes sequence GLAVFACLFLSTLLLVL.

In one aspect, the extracellular domain includes amino acids 1-68; 1-73 or 1-78 of SEQ ID NO:93. In one aspect, the intracellular domain includes amino acids 206-455 of SEQ ID NO:99. In one aspect, the trans-membrane domain includes sequence EDSGTTVLLPLVIFFGLCLLSLLFI.

In one aspect, the extracellular domain includes amino acids 1-68; 1-73 or 1-78 of SEQ ID NO:93. In one aspect, the intracellular domain includes amino acid residues 274-507 of SEQ ID NO:3. In one aspect, the trans-membrane domain includes sequence GLAVFACLFLSTLLLVL.

“Soluble forms” of polypeptides of the invention comprise certain fragments or domains of these polypeptides. Soluble polypeptides are polypeptides that are capable of being secreted from the cells in which they are expressed. A secreted soluble polypeptide can be identified (and distinguished from its non-soluble membrane-bound counterparts) by separating intact cells which express the desired polypeptide from the culture medium, e.g., by centrifugation, and assaying the medium (supernatant) for the presence of the desired polypeptide. The presence of the desired polypeptide in the medium indicates that the polypeptide was secreted from the cells and thus is a soluble form of the polypeptide. The use of soluble forms of cytokine polypeptides of the invention is advantageous for many applications. Purification of the polypeptides from recombinant host cells is facilitated, since the soluble polypeptides are secreted from the cells. Moreover, soluble polypeptides are generally more suitable than membrane-bound forms for parenteral administration and for many enzymatic procedures. In certain aspects of the invention, mature soluble forms of EPO-R or other polypeptides of the invention do not contain a trans-membrane or membrane-anchoring domain, or contain an insufficient portion of such a domain (e.g., 10 amino acids or fewer) to result in retention of the polypeptide in a membrane-bound form.

“An isolated polypeptide consisting essentially of an amino acid sequence” means that the polypeptide can optionally have, in addition to said amino acid sequence, additional material covalently linked to either or both ends of the polypeptide, said additional material between 1 and 10,000 additional amino acids covalently linked to either or both ends of the polypeptide; or between 1 and 1,000

additional amino acids covalently linked to either or both ends of the polypeptide; or between 1 and 100 additional amino acids covalently linked to either or both ends of the polypeptide. Covalent linkage of additional amino acids to either or both ends of the polypeptide according to the invention results in a combined amino acid sequence that is not naturally occurring.

“Correlation” and “correlation coefficient” are defined using the following formula:

$${ρ}_{X , Y} = \frac{cov ( X , Y )}{{σ}_{X} \cdot {σ}_{Y}}$$

$$where$$

$${σ}_{X}^{2} = 1 / n \sum {( {X}_{i} - {μ}_{X} )}^{2}$$

$$and$$

$${σ}_{Y}^{2} = 1 / n \sum {( {Y}_{i} - {μ}_{Y} )}^{2}$$

This correlation formula measures the relationship between two data sets that are scaled to be independent of the unit of measurement. The population correlation calculation returns the covariance of two data sets divided by the product of their standard deviations. The correlation coefficient, symbolized in the formula above as ρx,y, will also be referred to herein using the symbol “r”. The correlation formula determines whether two ranges of data move together—that is, whether large values of one set are associated with large values of the other (positive correlation or positive value of r, with a maximum value of r=1), whether small values of one set are associated with large values of the other (negative correlation or negative value of r, with a minimum value of r=−1), or whether values in both sets are unrelated (correlation near zero, or value of r equal to or near zero).

III. Receptor Polypeptides for Detection of Neutralizing Antibodies

A. Summary. Receptor polypeptides of the invention are polypeptides that comprise at least a portion of the extracellular domain of a receptor polypeptide or of a variant thereof, covalently linked to at least a portion of the intracellular domain of a receptor polypeptide or of a variant thereof. The extracellular domain and the intracellular domain portions can be derived from same or from different receptor polypeptides, including embodiments wherein the extracellular portion of the receptor is from the human receptor, for example, and the intracellular portion of the receptor is from the murine form of the receptor.

Examples of different receptors, of ligands that interact with them, and of genes that are up- or down-regulated in response to receptor-ligand interactions, are provided in Tables 7-21 below.

B. Extracellular Domains. The receptor polypeptide for crystallization comprises at least a portion of the extracellular region of the polypeptide of SEQ ID NO:1, SEQ ID NO:80, SEQ ID NO:92, or SEQ ID NO:109, or a variant thereof. In certain aspects, the entire extracellular region of the polypeptide of SEQ ID NO:1, SEQ ID NO:80, SEQ ID NO:92, or SEQ ID NO:109 is included in the receptor polypeptide. As certain examples, the receptor polypeptide can comprise amino acids 25 through 251 of SEQ ID NO:1, or amino acids 33 through 420 of SEQ ID NO:80, or amino acids 1 through 79 of SEQ ID NO:9, or a variant thereof.

C. Intracellular Domains. The receptor polypeptide for crystallization comprises at least a portion of the intracellular region of the polypeptide of SEQ ID NO:1, SEQ ID NO:80, SEQ ID NO:92, or SEQ ID NO:109, or a variant thereof. In certain aspects, the entire intracellular region of the polypeptide of SEQ ID NO:1, SEQ ID NO:80, SEQ ID NO:92, or SEQ ID NO:109 is included in the receptor polypeptide. As certain examples, the receptor polypeptide can comprise amino acids 275 through 509 of SEQ ID NO:1, or amino acids 445 through 801 of SEQ ID NO:80, or amino acids 101 through 189 of SEQ ID NO:9, or a variant thereof.

D. Variants. Another consideration that will guide those of skill in the art in making variants of receptor polypeptides is the nature of the amino acid substitutions that are made; such substitutions can be conservative, which means that the amino acid present in the variant at a certain position has the same chemical and/or size properties as the amino acid at the corresponding position in the unaltered receptor polypeptide. Table 1 summarizes groups of amino acids that are considered to have similar properties, so that the substitution of any amino acid with another from the same row of Table 1 would be a conservative substitution. In certain aspects, receptor polypeptide variants have 20% or fewer amino acid substitutions (or 15% or fewer, or 10% or fewer, or 7.5% or fewer, or 5% or fewer, or 2.5% or fewer, or 1% or fewer) across the length of polypeptides of the invention. In certain aspects, receptor polypeptide variants have 20% or fewer conservative amino acid substitutions (or 15% or fewer, or 10% or fewer, or 7.5% or fewer, or 5% or fewer, or 2.5% or fewer, or 1% or fewer) across the length of polypeptides of the invention.

In certain embodiments, the receptor polypeptides or variants thereof have EPO-binding activity, NGF-binding activity, BAFF-binding activity, or RANKL (OPG)-binding activity.

TABLE 1
Conservative Amino Acid Substitutions

| | |
|---|---|
| Basic: | arginine; lysine; histidine |
| Acidic: | glutamic acid; aspartic acid |
| Polar: | glutamine; asparagine |
| Alkyl: | leucine; isoleucine; valine; glycine; |
| | alanine |
| Aromatic: | phenylalanine; tryptophan; tyrosine |
| Small Non-Alkyl: | serine; threonine; cysteine; methionine; |
| | proline |

E. Expressing Receptor Polypeptides

The receptor polypeptides of the invention can be produced by living host cells that express the polypeptide, such as host cells that have been genetically engineered to produce the polypeptide. Methods of genetically engineering cells to produce polypeptides are well known in the art. See, e.g., Ausubel et al., eds. (1990), Current Protocols in Molecular Biology (Wiley, N.Y.). Such methods include introducing nucleic acids that encode and allow expression of the polypeptide into living host cells. These host cells can be bacterial cells, fungal cells, insect cells, or animal cells grown in culture. Bacterial host cells include, but are not limited to, Escherichia coli cells. Examples of suitable E. coli strains include: HB101, DH5α, GM2929, JM109, KW251, NM538, NM539, and any E. coli strain that fails to cleave foreign DNA. Fungal host cells that can be used include, but are not limited to, Saccharomyces cerevisiae, Pichia pastoris, and Aspergillus cells. A few examples of animal cell lines that can be used are CHO, VERO, BHK, HeLa, Cos, MDCK, 293, 3T3, and WI38. New animal cell lines can be established using methods well known by those skilled in the art (e.g., by transformation, viral infection, and/or selection).

Purification of the expressed receptor polypeptide can be performed by any standard method. When the receptor polypeptide is produced intracellularly, the particulate debris is removed, for example, by centrifugation or ultrafiltration. When the polypeptide is secreted into the medium, supernatants from such expression systems can be first concentrated using standard polypeptide concentration filters. Protease inhibitors can also be added to inhibit proteolysis and antibiotics can be included to prevent the growth of microorganisms. Receptor polypeptides can be produced in the presence of chaperone or accessory proteins in order to obtain a desired polypeptide conformation, or can be subjected to conditions such as oxidizing and/or reducing conditions after production in order to induce refolding or changes in polypeptide conformation (see, for example, WO 02/068455).

The receptor polypeptide can be purified using, for example, hydroxyapatite chromatography, gel electrophoresis, dialysis, and affinity chromatography, and any combination of purification techniques known or yet to discovered.

F. Gene Expression Methods

The invention provides methods of detecting compounds that affect therapeutic activity by measuring gene expression of response genes.

In one aspect, the expression of PIM-1, a protein serine/threonine kinase potentially involved in EPO-dependent survival and proliferation of erythroid precursors and other types of cells (Meeker et al. (1987) J. Cell. Biochem. 35: 105-12.; Wang et al., (2001) J Veterinary Sci. 2(3): 167-79; Kumenacker et al. (2001) J. Neuroimmunology 133: 249-259), and regulated by EPO in EPO-dependent UT-7 cells, can be used for detecting compounds affecting therapeutic activity of EPO. Previous studies have shown that PI3-K activity is critical for EPO-supported erythroid progenitor survival and differentiation (Kumenacker et al. (2001); Myklebust et al. (2002) Exp. Hematol. 30:990-1000; Uddin et al. (2000) Biochem. Biophys. Res. Comm. 275: 16-9; Sawyer and Jacobs-Helber, (2000) J Hematotherapy & Stem Cell Research 9:2 1-9). Data presented in Example 1 indicate that this signal is also essential for the EPO-dependent survival and/or proliferation of the UT-7 cells. Although the exact function of PIM-1 in EPO-stimulated cell survival and proliferation remains unclear, these observations suggest that it may be a signal transducer playing roles down stream of the PI3-K. At least, the expression of PIM-1 itself in EPO-dependent UT-7 cells is clearly coregulated by PI3-K signaling. Therefore, the level of PIM-1 mRNA expressed in EPO-dependent UT-7 cells reflects the amount of EPO signal received by the cells and can be used as a quantitative measurement for EPO signaling. Thus, the same strategy can be used for determining the presence and quantitative measurement of anti-EPO neutralizing antibodies that inhibit the biological activities of EPO.

In another aspect, PIM-1 expression can be used for detection of compounds, for example, neutralizing antibodies, that affect activity of therapeutic protein or antibodies/peptibodies by generating hybrid constructs using, for example, an intracellular domain of the EPO receptor linked to an extracellular domain of the receptor of interest. In one aspect, an intracellular domain of NGF receptor can be used. In another aspect, BAFF receptor can be used.

In another aspect, different response genes can be used. In one aspect, IL-8 expression can be used as a quantitative measurement. Thus, a construct using, for example, an intracellular domain of a TNF receptor and an extracellular domain of a receptor of interest can be created. In one aspect, a hybrid construct comprising the extracellular domain of BAFFR and the intracellular domain of TNFR can be created and thereby BAFF-induced IL-8 expression can be used to detect anti-BAFF neutralizing antibody, for example.

In yet another aspect, mRNA expression of the terminal differentiation marker TRAP (tartrate-resistant acid phosphatase) can be used (Lacey et al. (1988)). The inhibition of OPG ligand/RANK by antibodies or peptibodies would inhibit TRAP production as well, however, if compounds affecting anti-RANK antibodies were present, such as neutralizing antibodies, the TRAP enzyme would continue to be produced.

The above description of the invention is exemplary, and is not meant to be limiting as to, for example, the response gene, the types of host cells used, the methods and compositions for providing for host cells having varying levels of expression of a response gene, and the like, as such can be varied and remain within scope of the present invention, and such variations will be readily appreciated by the ordinarily skilled artisan.

G. Branched DNA Technology Expression of a response gene can be detected in a variety of ways well known in the art, e.g., by use of hybridization probes, PCR primers, or antibodies specific for a response gene product.

In one aspect, branched DNA (bDNA technology) can be used to quantitatively measure expression of a response gene. This is a highly sensitive and convenient to use technique. Urdea, M. and Wuestehube, L. (2000). Branched DNA (bDNA) technology. In: C. Kessler (Ed.) Nonradioactive Analysis of Biomolecules. Srpinger-Verlag Press & Publications, Heidelberg, p. 388. It can detect the existence of as few as 1 to 50 copies of mRNA in a sample. Elbeik, T. et al. (2000) J. Clin. Microbiol. 38: 1113-20. The entire procedure can be fully automated, making it a good choice for high throughput operations. Murphy, D. G. et al. (2000) J. Clin. Microbiol. 38: 4034-41.

The above description of the invention is exemplary, and is not meant to be limiting as to, for example, the methods for detecting mRNA of the response gene, and the like, as such can be varied and remain within scope of the present invention, and such variations will be readily appreciated by the ordinarily skilled artisan.

For example, rather than detect changes in expression of a response gene by using bDNA technology, response gene expression can be detected by using methods well known in the art for detecting gene expression levels (e.g., Northern blot, microarray-based, or other solid support-based methods, tailored expression membrane assays, or Taqman assays etc.). Also it will be readily appreciated that increasing the number of response genes analyzed (e.g., two or more response genes) can provide additional information and/or increase confidence scores of the results obtained relative to detection of a single response gene. Assays with multiple response genes can be conducted simultaneously using different detectable labels for each gene (e.g., different fluorescent reporters having different excitation and/or emission wavelengths).

H. Introduction into Host Cells

Methods for introducing a construct into a host cell are well known in the art. This can be accomplished by, for example, introduction of an autonomous plasmid, which can be maintained as an episomal element and/or chromosomally integrated into the genome of the host cell. Suitable constructs, vectors, plasmids, etc. are well known in the art and will vary with the host cell, size and other characteristics of the reporter gene, etc.

The methods of the invention can be used in connection with any of a variety of host cells, including eukaryotic, prokaryotic, diploid, or haploid organisms. Host cells can be single cell organisms (e.g., bacteria) or multicellular organisms (transgenic organisms, such as insects (e.g., Drosophila spp), worms (e.g., Caenorhabditis spp, e.g., C. elegans) and higher animals (e.g., transgenic mammals such as mice, rats, rabbits, hamsters, humans etc. or cells isolated from such higher animals, including humans). The host cell can also be a cell infected with a virus or phage that contains a target sequence in the viral or phage genome.

The following examples are offered to more fully illustrate the invention, but are not to be construed as limiting the scope thereof.

### EXAMPLE 1

This example illustrates an assay which measures the variations of target gene expression that reflect the biologic effect of a therapeutic agent and capabilities of the antibodies, if present, to neutralize the therapeutics. In particular, this method can be used for detection and measurement of anti-erythropoietin antibodies.

Cells and Proteins

UT-7, a human acute megakaryocytic leukemia cell line was maintained in growth media [RPMI/1640 (Gibco, N.Y.) containing 10% fetal calf serum (Hyclone, Logon, Utah)] supplemented with 10 ng/mL granulocyte-macrophage colony-stimulating factor (GM-CSF). Recombinant human erythropoietin (rEPO), stem cell factor (rSCF), granulocyte colony-stimulating factor (rG-CSF), rGM-CSF, mouse interleukin-3 (rIL-3), and rabbit anti-human EPO polyclonal antibody (29123) were provided by Amgen. Protein kinase inhibitors for phosphatidylinositol 3-kinase (PI3-K) (LY294002), MAP kinase (MAPK) (UO126), protein kinase A (PKA), and protein kinase C (PKC) were all purchased from Promega (Madison, Wis.).

Microarray Analysis of Gene Expression

Microarray analysis of cell samples was performed using well-described protocols (Eisen and Brown (1999) Methods Enzymol. 303, 179-205) with minor modifications. PolyATtract (Promega) purified mRNA was reverse-transcribed using random primers in the presence of either Cy3 or Cy5 dye-labeled dCTP. Control and test fluorescent probes were hybridized to cDNA-spotted glass slides overnight in a competitive hybridization process. After washing, fluorescent images of the dried slides were obtained using a GenePix Scanner 4000 (Axon Instruments, Union City, Calif.). GenePix Pro 3.0 software (Axon Instruments, Union City, Calif.) was used for feature detection. The subsequent data were inspected using a series of internal standards that enabled determination of sensitivity, linearity, and dynamic range of response within individual experiments. Global normalized data was then exported into the Resolver (Rosetta, Kirkland, Wash.) database for storage and analysis. For each control and test pair, data was combined with a second dye-swapped hybridization to reduce potential dye incorporation biased measurements.

Oligonucleotide Probes

Human PIM-1 specific probes and human cyclophilin probes for bDNA analysis were designed by using the ProbeDesigner software from Bayer Corporation (West Haven, Conn.). Three sets of oligonucleotide probes were designed for each molecule: the capture extender (CE), label extender (LE), and blocker (BL). Thirty-one probes were generated for PIM-1, including 8 CE probes, 17 LE probes, and 6 BL probes; and 27 were made for cyclophilin, including 6 CE probes, 18 LE probes, and 3 BL probes (Table 2). All probes for each gene were pooled according to the manufacturer's instructions.

TABLE 2
Oligonucleotide probes for bDNA detection

Human PIM-1

| | |
|---|---|
| CE | gctcgggcctctccaggTTTTTCTCTTGGAAAGAAAGT |
| | agtcgaagagatcttgcaccgTTTTTCTCTTGGAAAGAAAGT |
| | ggccagctcctcttgcaggTTTTTCTCTTGGAAAGAAAGT |
| | agctcgccgcgattgagTTTTTCTCTTGGAAAGAAAGT |
| | ttgagcagcgcccccgaTTTTTCTCTTGGAAAGAAAGT |
| | catacagcaggatccccaggTTTTTCTCTTGGAAAGAAAGT |
| | attctgaagagaccctctgcctgaaTTTTTCTCTTGGAAAGAAAGT |
| | gatttcttcgaaggttggcctTTTTTCTCTTGGAAAGAAAGT |
| LE | gcaggaccacttccatgggTTTTTAGGCATAGGACCCGTGTCT |
| | acccgagctcaccttcttcaTTTTTAGGCATAGGACCCGTGTCT |
| | gcctaatgacgccggagaaTTTTTAGGCATAGGACCCGTGTCT |
| | cctctcgaaccagtccaggaTTTTTAGGCATAGGACCCGTGTCT |
| | atcaggacgaaactgtcgggTTTTTAGGCATAGGACCCGTGTCT |
| | gctcccctttccgtgatgaTTTTTAGGCATAGGACCCGTGTCT |
| | gccgcacggcctccagTTTTTAGGCATAGGACCCGTGTCT |
| | ccccgcagttgtggcagtTTTTTAGGCATAGGACCCGTGTCT |
| | tgatgtcgcggtgtagcaTTTTTAGGCATAGGACCCGTGTCT |
| | gtcgataaggatgttttcgtcctTTTTTAGGCATAGGACCCGTGTCT |
| | aagtccgtgtagacggtgtccTTTTTAGGCATAGGACCCGTGTCT |
| | ctatacactcgggtcccatcgTTTTTAGGCATAGGACCCGTGTCT |
| | ctgccatggtagcgatggtaTTTTTAGGCATAGGACCCGTGTCT |
| | gaccagactgccgccgacTTTTTAGGCATAGGACCCGTGTCT |
| | aaggaatatctccacacaccatatTTTTTAGGCATAGGACCCGTGTC |
| | T |
| | agcaccatctaatgagatgctgacTTTTTAGGCATAGGACCCGTGTC |
| | T |
| | ttgcatccatggatggttctgTTTTTAGGCATAGGACCCGTGTCT |
| BL | cacctgccagaagaagctgcg |
| | cccgaagtcgatgagcttg |
| | gcggatccactctggaggg |
| | gatctcttcgtcatgctcga |
| | gaaaacctggcccctgat |
| | atctgatggtctcagggcca |
| | Human Cyclophilin |
| CE | atacaccttgacggtgactttgTTTTTCTCTTGGAAAGAAAGT |
| | cctttctctcctgtagctaaggcTTTTTCTCTTGGAAAGAAAGT |
| | ggtgtctttgcctgcgttgTTTTTCTCTTGGAAAGAAAGT |
| | tgaagaactgggagccgttTTTTTCTCTTGGAAAGAAAGT |
| | gtctgtcttggtgctctccaTTTTTCTCTTGGAAAGAAAGT |
| | tcaggggtttatcccggctTTTTTCTCTTGGAAAGAAAGT |
| LE | gccgcagaaggtcccggcTTTTTAGGCATAGGACCCGTGTCT |
| | ggccccttcttcttctcatcgTTTTTAGGCATAGGACCCGTGTCT |
| | catctccaattcgtaggtcaaaTTTTTAGGCATAGGACCCGTGTCT |
| | agatcacccggcctacatcttTTTTTAGGCATAGGACCCGTGTCT |
| | cacaaaattatccactgtttttggTTTTTAGGCATAGGACCCGTGTC |
| | T |
| | atttgctgtttttgtagccaaatTTTTTAGGCATAGGACCCGTGTCT |
| | agtctccgccctggatcatTTTTTAGGCATAGGACCCGTGTCT |
| | tgtgccatctcccctggtgaTTTTTAGGCATAGGACCCGTGTCT |
| | accgtagatgctctttcctccTTTTTAGGCATAGGACCCGTGTCT |
| | agttctcatcggggaagcgctTTTTTAGGCATAGGACCCGTGTCT |
| | aggcccgtagtgcttcagtttgaTTTTTAGGCATAGGACCCGTGTCT |
| | gccatgctgacccagccTTTTTAGGCATAGGACCCGTGTCT |
| | acatgcttgccatctagccaTTTTTAGGCATAGGACCCGTGTCT |
| | ccctctagaactttgccaaacaccTTTTTAGGCATAGGACCCGTGTC |
| | T |
| | ccttccgcaccacctccatgTTTTTAGGCATAGGACCCGTGTCT |
| | cagtctgcgatgatcacatcctTTTTTAGGCATAGGACCCGTGTCT |
| | tccacctcgatcttgccgTTTTTAGGCATAGGACCCGTGTCT |
| | gcgatggcaaagggcttcTTTTTAGGCATAGGACCCGTGTCT |
| BL | aacagtctttccgaagagaccaa |
| | gaagtccttgattacacgatgga |
| | ggctgtcttgactgtcgtga |

Cell Treatment

UT-7 cells were washed 2 times with the growth media and incubated overnight in rGM-CSF-free media at 37° C. with 5% CO₂. Triplicate samples of the rGM-CSF-starved cells were seeded in 96-well tissue culture plates with 100 μL rGM-CSF-free media at a density of 1.2×10⁵ cells per well and treated with various concentrations of rEPO in the absence or presence of anti-EPO antibody, or with various concentrations of other cytokines, including rGM-CSF, rG-CSF, rSCF, and rIL-3, at 37° C. for 90 minutes.

For serum tolerate experiments, GM-CSF-starved UT-7 cells were either treated with 3 ng/mL rEPO in the presence or absence of various concentrations pooled normal human serum (PNHS, Bioreclamation, Inc., East Meadow, N.Y.) or with various concentrations of rEPO in the presence or absence of 10% PNHS at 37° C. for 90 minutes.

For studies using signal transduction antagonists, the rGM-CSF-starved cells were first treated with various concentrations of LY294002, UO126, and the inhibitors for PKA and PKC separately at 37° C. for 30 minutes and then with 21 ng/mL of rEPO at 37° C. for 90 minutes or 24 hours. After all treatments, the levels of PIM-1 expression were determined using branched DNA (bDNA) technology. The number of cells in each well that had been treated with rEPO for 24 hours was counted.

Branched DNA Analysis

Branched DNA analysis was performed using the QuantiGene High Volume Kit (Bayer, West Heaven, Conn.) using a 3-step procedure provided by the manufacturer, which included specimen preparation, hybridization, and detection. Briefly, treated or untreated UT-7 cells seeded in 96-well tissue culture plates were mixed with 50 μL lysis mixture (provided by the kit) using a multiple channel pipette and incubated at 46° C. for 30 minutes to release mRNA. Aliquots of 70 μL and 30 μL of each lysate were transferred to capture plates (provided by the kit) with 30 μL pooled PIM-1-specific probes or 70 μL pooled cyclophilin probes, respectively, and incubated overnight at 53° C. The hybridization mixtures were removed and the plates were washed twice with 400 μL wash buffer (0.1 SSC, 0.03% lithium lauryl sulfate) using an Auto Plate Washer (Bio-TEK, Winooski, Vt.). After washing, 100 μL Amplifier Working Reagent (provided by the kit) was added to each well and incubated at 46° C. for 1 hour. The plates were washed twice as described above, incubated with 100 μL Labeling Working Reagent (provided by the kit) at 46° C. for 1 hour, washed again 3 times, and processed for chemiluminescent detection. The amount of the target mRNA in each sample was determined by the intensity of the luminescent emission detected using a luminometer (Wallac Victor 1420, Perkin Elmer, Finland).

[³H]-Thymidine Incorporation

UT-7 cells were washed 2 times with growth media and incubated overnight in rGM-CSF-free media at 37° C., with 5% CO₂ as described above. Triplicate samples of the rGM-CSF-starved cells were seeded in 96-well tissue culture plate at a density of 1×10⁵ cells per well and treated with various concentrations of rEPO in the presence or absence of anti-EPO antibody at 37° C. with 5% CO₂ for 72 hours. Then, 2 μCi [³H]-thymidine (Amersham, Little Chalfont, Buckinghamshire, UK) were added to each well and the cells were further incubated for 4 hours. The cells were harvested using a cell harvester (Filtermate 196, Packard, Ill.), and the incorporated radioactivity was determined using a Matrix 9600 beta counter (Packard, Ill.).

EPO Induces PIM-1 Expression

mRNA microarray experiments determined which genes in UT-7 cells had altered expression after rEPO treatment. UT-7 cells quieted in rGM-CSF-free media were treated with 20 ng/mL rEPO at 37° C. for 2, 4, 6, or 24 hours. Messenger RNAs extracted from the rEPO-treated or untreated control cells were used to generate probes for the subsequent microarray experiments. The mRNA expression level of a number of genes has been changed in rEPO-treated cells compared with that in the untreated control cells (Table 3). In particular, the level of the PIM-1 mRNA in rEPO-treated cells was more than 20 times higher than that in the untreated control cells.

TABLE 3
Epo-Altered mRNA expression in UT-7 cells

Folds*

| Gene Name | 2 hr | 4 hr | 6 hr | 24 hr |
|---|---|---|---|---|
| PIM 1 | +8.06 | +13.85 | +8.49 | +21 |
| LOC64182 | +2.96 | +6.42 | +3.98 | +13.68 |
| RTP801 | +2.47 | +3.75 | +3.68 | +9.64 |
| NFYC | +1.48 | +2.07 | +2.39 | +8.25 |
| CBS | +1.43 | +1.47 | +1.66 | +7.59 |
| IGFBP4 | +2.14 | +2.91 | +2.82 | +5.57 |
| ATF5 | +1.12 | +1.53 | +1.67 | +5.22 |
| CTH | +1.32 | +1.97 | +2.66 | +5.16 |
| PKM2 | +1.30 | +1.43 | +1.52 | +5.15 |
| LDHA | +1.31 | +1.83 | +2.24 | +4.80 |
| JTB | +1.42 | +1.82 | +2.53 | +4.33 |
| GPI | +1.32 | +1.47 | +1.50 | +4.24 |
| VEGF | +1.88 | +2.05 | +2.32 | +3.81 |
| ATF4 | +1.85 | +1.95 | +2.39 | +3.60 |
| LDHC | +1.35 | +1.63 | +2.41 | +3.31 |
| TIMP1 | +1.50 | +1.50 | +1.60 | +3.16 |
| POLD1 | −2.45 | −1.35 | +1.13 | −2.18 |
| GCH1 | −2.81 | −1.14 | +1.08 | −2.21 |
| EIF4G3 | −1.25 | −1.33 | −1.40 | −2.48 |
| SPTA1 | −1.83 | −1.77 | −1.74 | −2.64 |
| RYR3 | −1.79 | −1.91 | −1.80 | −2.83 |
| MGLL | −2.77 | −1.12 | 1.09 | −284 |
| AKR1C3 | −1.01 | −1.08 | −1.13 | −2.92 |

*Represent the ratio of the mRNA levels in EPO-treated cells versus that in the untreated cells

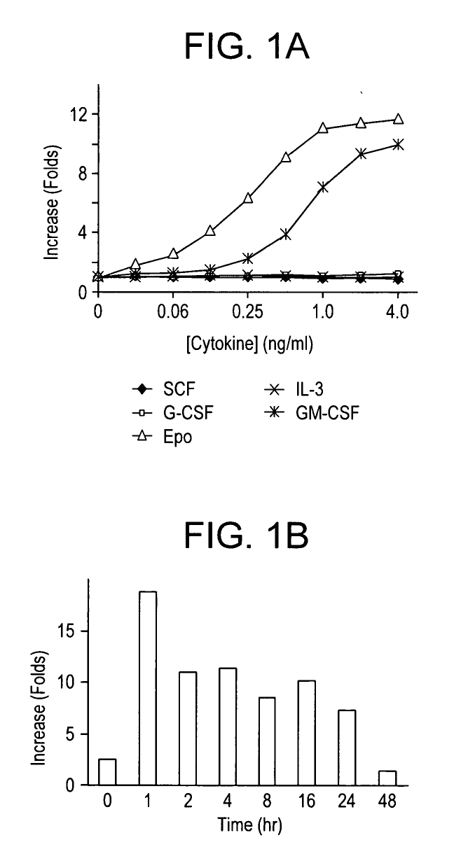

The EPO upregulated PIM-1 mRNA expression in UT-7 cells was further confirmed using bDNA technology (FIG. 1A). Similar to rEPO, rGM-CSF also induced expression of the PIM-1 mRNA in UT-7 cells (FIG. 1A), while rSCF, rG-CSF, and IL-3 showed no effect. The magnitude of the rEPO-induced PIM-1 expression in UT-7 cells was dose dependent and the maximum induction was reached after 90 minutes of rEPO treatment (FIG. 1B).

PIM-1 Expression is Regulated by PI3K Signaling

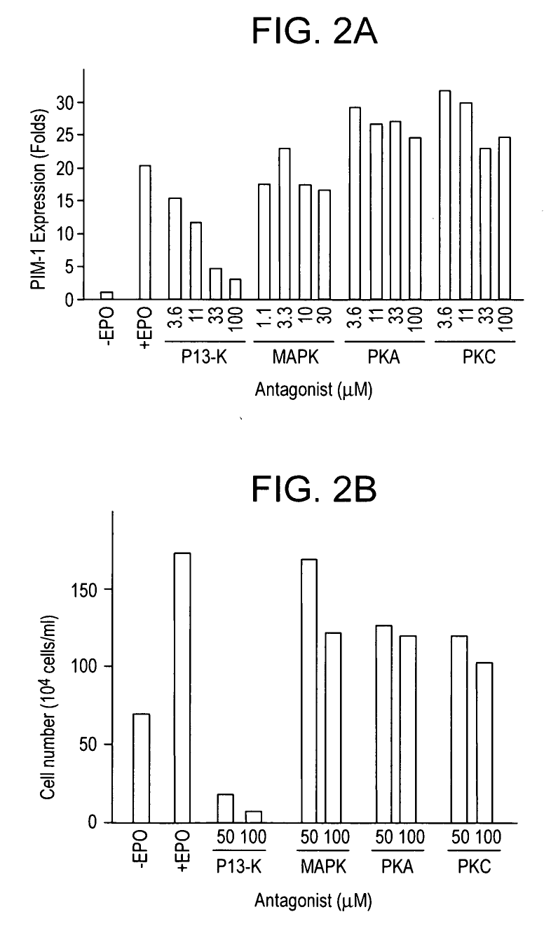

Chemical antagonists of the major intracellular signal transduction molecules downstream of the EPO receptor were used to determine which of the EPO signaling pathways were involved in the up-regulated PIM-1 expression. LY294002, a PI3-K inhibitor, effectively inhibited EPO-induced PIM-1 expression after 30 minutes of pre-incubation and the inhibition was apparently in a dose-dependent manner (FIG. 2A). All other antagonists showed either no or very mild effects on the regulated PIM-1 expression. When cells were treated for 24 hours, LY294002 blocked EPO-induced UT-7 cell proliferation and appeared to have activated apoptosis of the cells (FIG. 2B).

Effects of Serum on EPO-Induced PIM-1 Expression

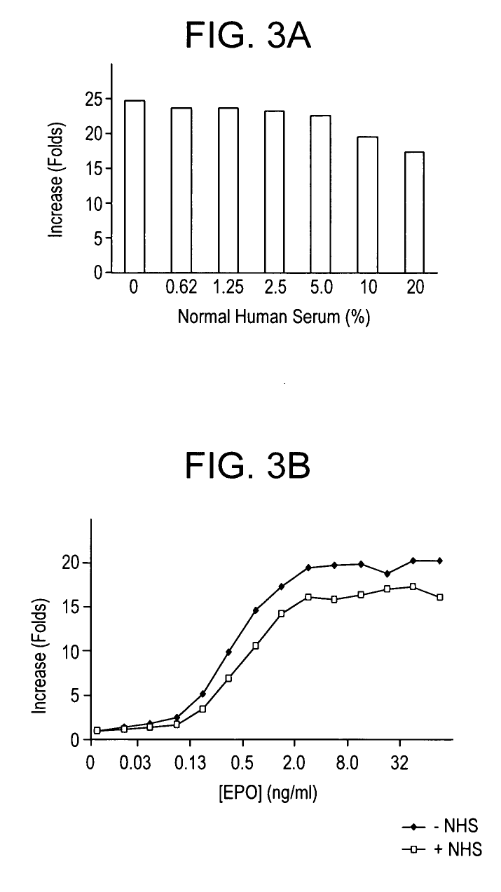

To determine if PIM-1 expression could be used as a biological measurement for detecting neutralizing antibodies in serum samples, the effect of serum concentrations on EPO-induced PIM-1 expression were evaluated. As shown in FIG. 3, up to 20% of PNHS was well tolerated by this assay system.

Detection of Anti-EPO Neutralizing Antibodies in Serum

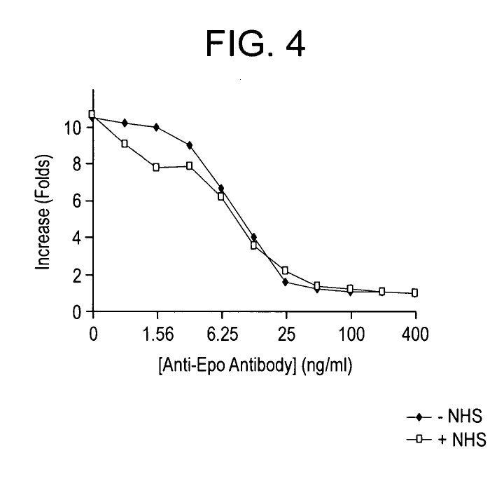

The rabbit polyclonal antibody 29123 is an EPO specific neutralizing antibody that has been shown to inhibit the survival and proliferation of UT-7 cells in media supplemented with rEPO. In these experiments, this antibody effectively inhibited EPO-induced elevation of PIM-1 expression (FIG. 4). The inhibition was dose dependent and was not affected by the presence of 10% normal human serum. Apparent inhibition could be observed at 2 ng/mL of 29123, and the maximum effect was reached at approximately 30 ng/mL.

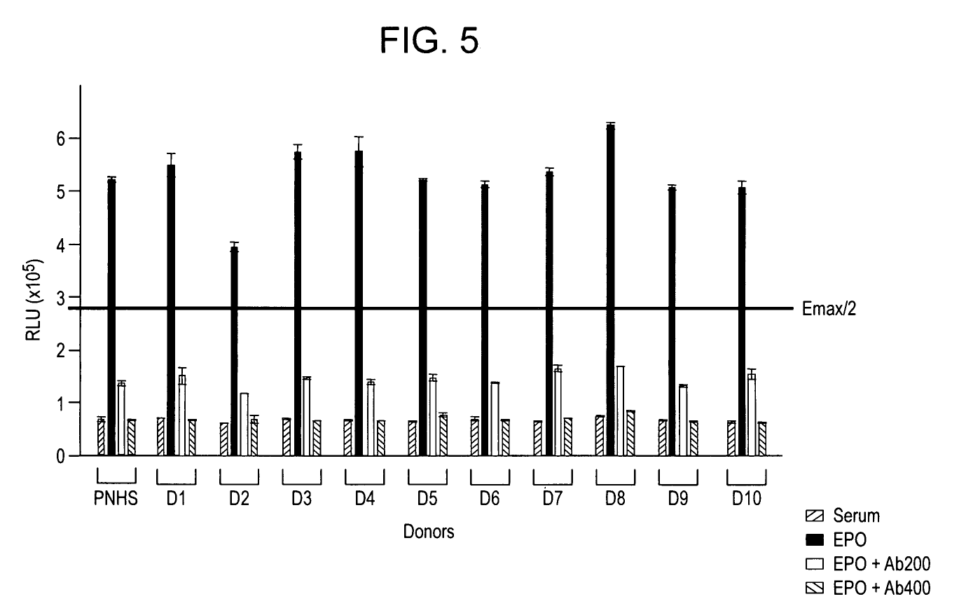

The feasibility of using PIM-1 expression as a biological measurement for detecting anti-EPO neutralizing antibodies in serum samples was further evaluated by a more realistic spiking experiment. The levels of PIM-1 expression in cells treated with each donor serum only were compared with expression levels of the cells treated with the serum plus rEPO or rEPO and the spiked antibody. When 400 ng/mL antibody was spiked into the donor sera, EPO-induced PIM-1 expression was almost completely blocked by all of the spiked samples (FIG. 5). When 200 ng/mL antibody was spiked, however, effective, but weaker inhibition was observed. In either case, the levels of PIM-1 expressed in all antibody-treated samples were well below the tentative cutoff line for positive assignment. Additionally, the result indicated once again that PIM-1 expression was not influenced by the presence of at lease 10% of human serum, implying that a PIM-1 expression-based assay system could tolerate assay matrixes with high concentrations of serum for potential improvement of assay sensitivities.

Gene Expression Vs [³H]-Thymidine Incorporation

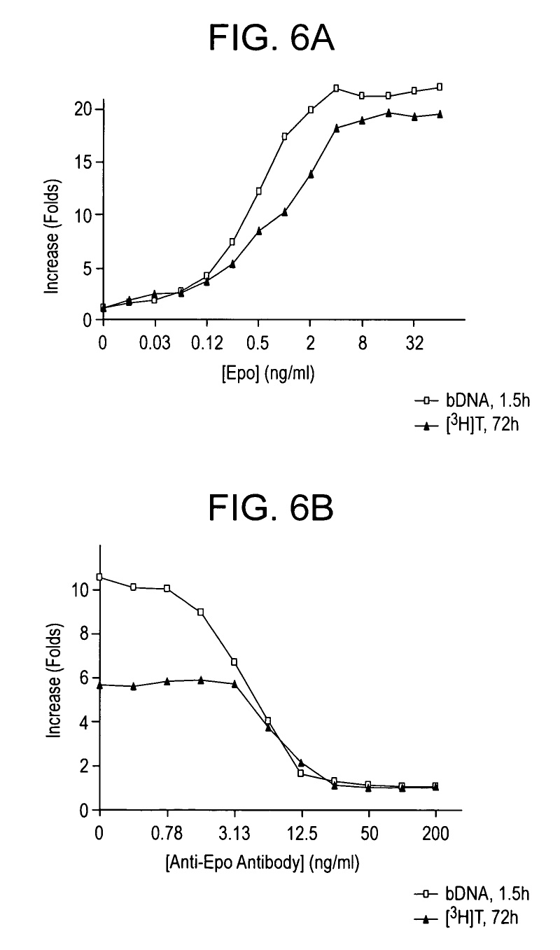

As shown in FIG. 6A, the pattern of EPO-induced PIM-1 expression was almost superimposable to that of EPO-induced [³H]-thymidine incorporation in UT-7 cells. The Pearson's correlation coefficient between the increased PIM-1 expression and [³H]-thymidine incorporation was 0.974, confirming that these 2 events were highly related. The maximum induction and the EC₅₀ for EPO-induced PIM-1 expression were approximately 22-fold and 0.4 ng/mL, while the same for [³H]-thymidine incorporation were approximatelyl 9-fold and 1 ng/mL. The blocking of EPO-induced PIM-1 expression and that of [³H]-thymidine incorporation by anti-EPO antibodies were also highly related (FIG. 6B). The Pearson's correlation coefficient between these 2 processes was 0.958. The maximum inhibition of PIM-1 expression and [³H]-thymidine incorporation were approximately 11-fold and 6-fold; while the IC₅₀ for PIM-1 expression and [³H]-thymidine incorporation were approximately 3.4 ng/mL and 5.4 ng/mL, respectively (FIG. 6). These numbers indicated that the gene expression approach was more robust and sensitive than the [³H]-Thymidine incorporation method.

### EXAMPLE 2

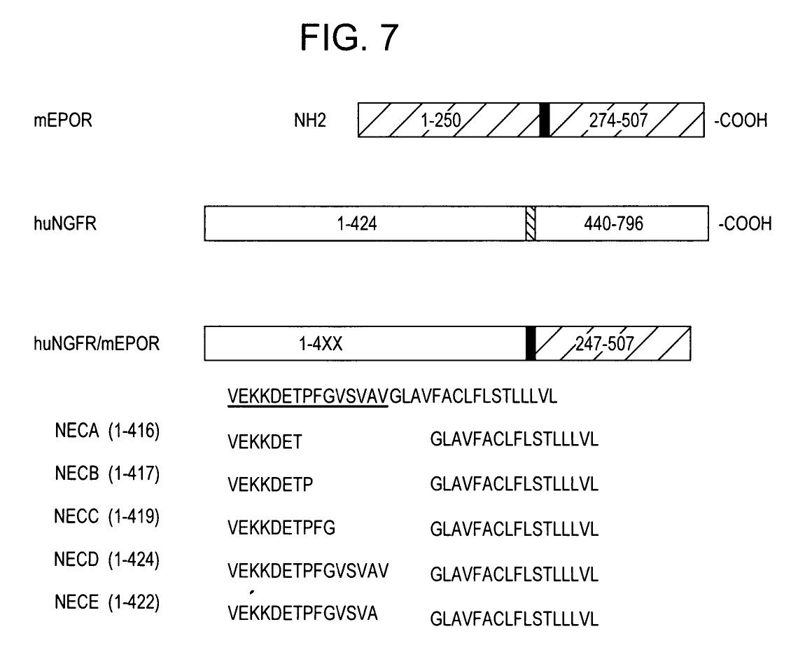

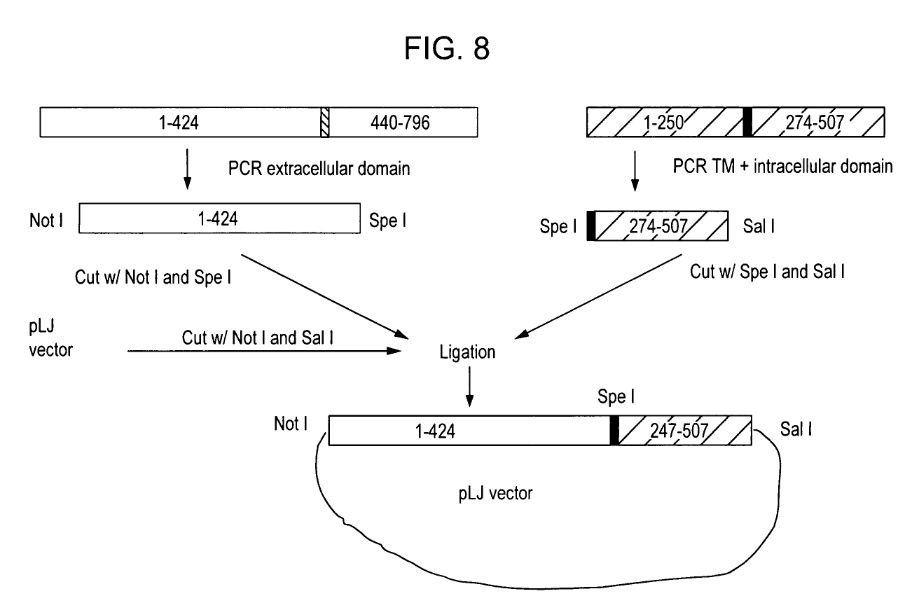

This example illustrates the application of the method to NGFR (Nerve growth factor receptor) and EPOR hybrid receptors. Briefly, five different human NGFR and EPOR hybrid receptors (NECA-NECE, NGFR/EPOR chimera A-E) have been constructed. Different lengths of extracellular domain of human NGFR were fused with the mouse EPO receptor trans-membrane and intracellular domains (FIG. 7). The amino acid sequence at the bottom of FIG. 7 represents the junction points of hybrid receptors, wherein the trans-membrane domain is italicized and the 3′ end of NGFR extracellular sequence is underlined. The black bars indicate the sequence position where the 3′-end of the NGF was fused to EPOR in each chimeric construct. FIG. 8 represents a generic cloning strategy for making NGFR/EPOR hybrid receptors. Human NGF receptor extracellular domain fragment was obtained by PCR followed by restriction enzyme digestion by Not I at 5′ end and Spe I at 3′ end. A fragment containing mouse EPOR trans-membrane and intracellular domains was obtained by PCR followed by digestion by Spe I at 5′ end and Sal at 3′ end. Two fragments were ligated and subcloned into the pLJ vector cut with Not I and Sal I. Positive clones were obtained and sequenced to confirm sequence.

These five different forms of NGFR/EPOR hybrid receptor constructs were transfected into 32Dcl3 cell via electroporation and were selected by medium containing G418 and NGF to yield 32D/NECD cells. A NGF responsive cell line NECDsc-14 was generated after two rounds of selection and single cell subcloning. These 32D/NECDsc-14 cells were maintained in either 5 ng/ml mouse interleukin-3 (mIL-3) or 25 ng/ml of NGF. NGF induced NECDsc-14 cell proliferation can be measured by [3H]-uptake. Cells were washed three times, staged overnight with growth factor-free culture medium, and treated with various amount of mIL-3 (control) or NGF (as indicated in Table 4) for 18 hours. The amounts of [³H]-thymidine incorporated into the cells were measured following further incubation of the cells with [³H]-thymidine for 4 hours. Results of this experiment are represented in Table 4 below.

TABLE 4
NGF Dose Response

| Counts | | |
|---|---|---|
| Conc | NECDsc-14 | NECDsc-14 |
| (ng/ml) | mIL-3-dependent | NGF-dependent |
| 0.00 | 655.3 | 1209.3 |
| 0.02 | 1724.7 | 1963.3 |
| 0.07 | 3784.0 | 4691.0 |
| 0.21 | 8645.7 | 8033.0 |
| 0.62 | 18964.0 | 16838.0 |
| 1.85 | 28205.7 | 22404.0 |
| 5.56 | 32071.3 | 24205.3 |
| 16.67 | 31791.3 | 22907.0 |
| 50.00 | 30813.7 | 21618.3 |
| 150.00 | 32221.7 | 20857.3 |

This NGF induced proliferation of NECDsc-14 cells can be used to detect anti-NGF neutralizing antibodies or peptibodies, which inhibit the NGF-induced proliferation of NECDsc-14 cells, in biological samples similar to the assay described in Example 1. Similarly, it can be used to detect neutralizing antibodies against the anti-NGF antibodies or peptibodies, which reverse the inhibitory effects mentioned above, in biological samples. Assays for detecting and measuring the concentrations of neutralizing antibodies against anti-NGF antibodies or peptibodies can be performed in 1% human serum, 5% cynomolgus monkey serum, or 2% rat serum samples (see Table 5), as no significant matrix effect from these samples was observed.

TABLE 5
NGF Dose Response in Serum Matrix

| NGF conc. | | 5% cyno | 2% rat | 1% human |
|---|---|---|---|---|
| (ng/mL) | No serum | serum | serum | serum |
| 0.00 | 462.7 | 1534.7 | 702.7 | 367.3 |
| 0.02 | 595.3 | 2175.7 | 1188.3 | 946.7 |
| 0.07 | 1389.7 | 5761.7 | 3524.0 | 1480.0 |
| 0.21 | 5423.0 | 15774.0 | 11970.7 | 5832.3 |
| 0.62 | 17073.0 | 32462.7 | 29431.7 | 13374.7 |
| 1.85 | 27445.3 | 43119.7 | 37695.3 | 19493.7 |
| 5.56 | 35224.7 | 52240.7 | 43395.7 | 22160.0 |
| 16.67 | 39675.3 | 51793.3 | 42446.0 | 24558.7 |
| 50.00 | 41018.0 | 56389.7 | 46874.3 | 19712.0 |
| 150.00 | 44119.0 | 62140.0 | 49545.3 | 18626.7 |

### EXAMPLE 3

This example illustrates the application of the method to detecting the presence and measuring the concentration of neutralizing antibodies against an anti-BAFF antibody or peptibody. In particular, it is demonstrated that a BAFF-induced release of IL-8 (interleukin-8) that can be measured by ELISA can serve to identify and measure the cellular response to BAFF (B cell Activating Factor).

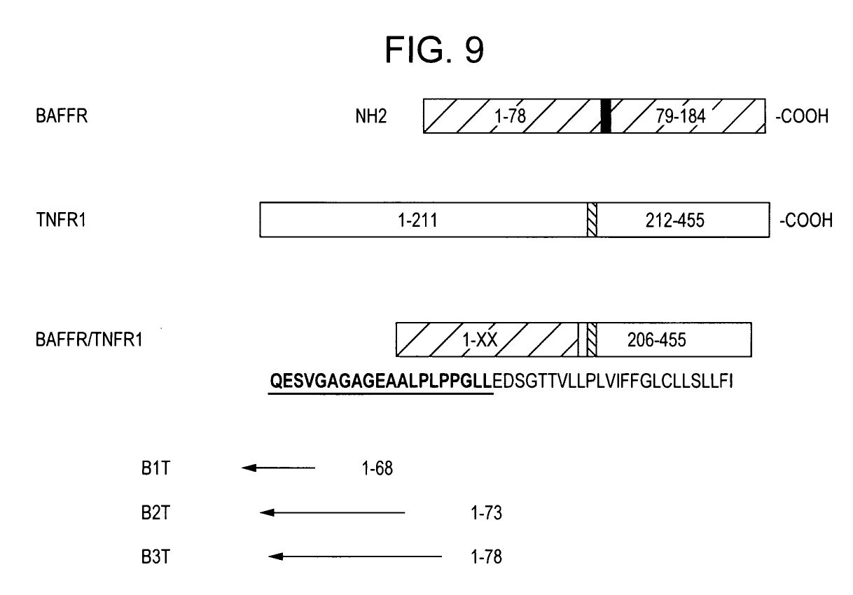

To measure the release of IL-8, a BAFF/TNFR hybrid receptor was constructed. As demonstrated in FIG. 9, three different human BAFFR and human TNFR hybrid receptors (B1T-B3T, BAFFR/TNFR hybrid receptors 1-3) were constructed. Extracellular domains of the human BAFF receptor of different length (BMCA, 68 amino acids; BMCB, 73 amino acids; BMCC, 78 amino acids) were fused with the human TNF receptor domain consisting of amino acids 206-455. The amino acid sequence in FIG. 9 represents the junction point of the hybrid receptor, wherein the trans-membrane domain area of the TNF is italicized and the 3′ end of the BAFFR extracellular domain is underlined.

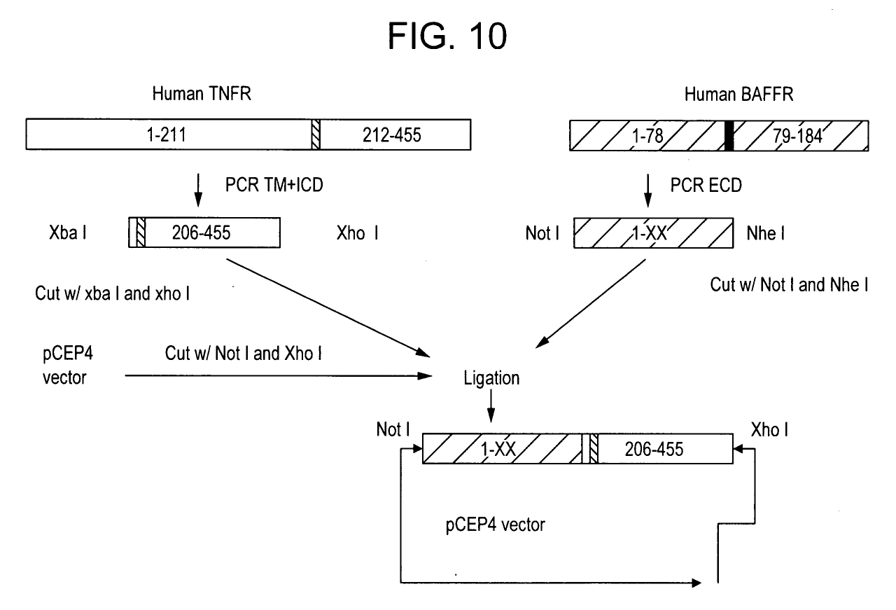

FIG. 10 outlines a generic cloning strategy for making BAFF/TNFR hybrid receptors. Briefly, a fragment of an extracellular domain of the human BAFFR was obtained by PCR followed by restriction enzyme digestion by Not I at the 5′ end and Nhe I at the 3′ end. A fragment including the trans-membrane and the intracellular domains of the human TNFR was obtained by PCR and restriction enzyme digestion by Xba I at 5′ end and Xho I at the 3′ end. Two fragments were ligated and subcloned into the pCEP4 vector cut with Not I and Xho I. Positive clones were obtained and sequenced to confirm sequence.

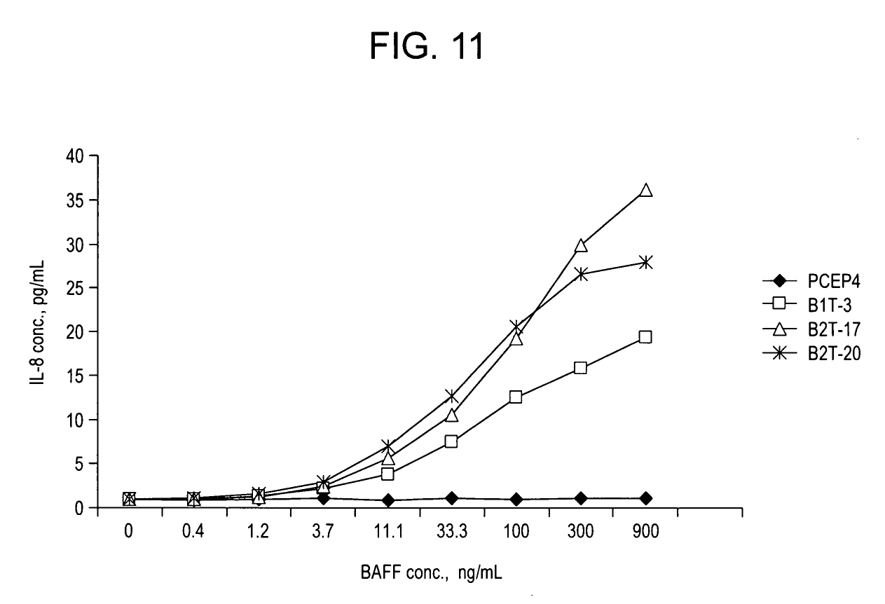

COS-1 cells were stably transfected with the BAFFR/TNFR chimeric constructs and selected in Hygromycin to yield clones of the hybrid receptor expressing COS-1 cells. The final clones of the cells expressing the BAFF/TNFR chimera were tested in a BAFF induced IL-8 release from COS-1 cells expressing BAFFR/TNFR hybrid. Briefly, COS-1 cells were transfected stably with the BAFFR/TNFR chimeric construct (B2T) and were selected in Hygromycin to yield COS-1/B2T cells. FIG. 11 illustrates the IL-8 production (in pg/ml) by cell lines PCEP4, B1T-3, B2T-17, and B2T-20 FIG. 11 illustrates the IL-8 production (in pg/ml) by cell lines PCEP4, B1T-3, B2T-17, and B2T-20. The cells were then seeded in a 12-well plate and treated with BAFF at indicated concentrations at 37^{o}C for 18 hours. The conditioned medium was collected and the IL-8 production was measured by ELISA kit (R&D systems). Because of the highest IL-8 production, B2T-17 line was selected for the rest of the study.

This BAFF-induced IL-8 expression from B2T-17 cells can be used in biological sample to detect anti-BAFF neutralizing antibody or peptibody, which inhibits the BAFF-induced IL-8 release from B2T-17 cells. Similar to he assay described in Example 1, the same construct can be used to detect neutralizing antibodies against the anti-BAFF antibodies or peptibodies, which reverse the inhibitory effects mentioned above. Assays for detecting and measuring the amount of neutralizing antibodies against anti-BAFF antibodies or peptibodies can be performed in 1% human or 5% cynomolgus monkey serum, as no significant matrix effect from these samples was observed.

### EXAMPLE 4

This example illustrates the application of the method to detecting the presence and measuring the concentration of neutralizing antibodies against an anti-BAFF antibody or peptibody by measuring an alteration in the BAFF-induced PIM-1 expression.

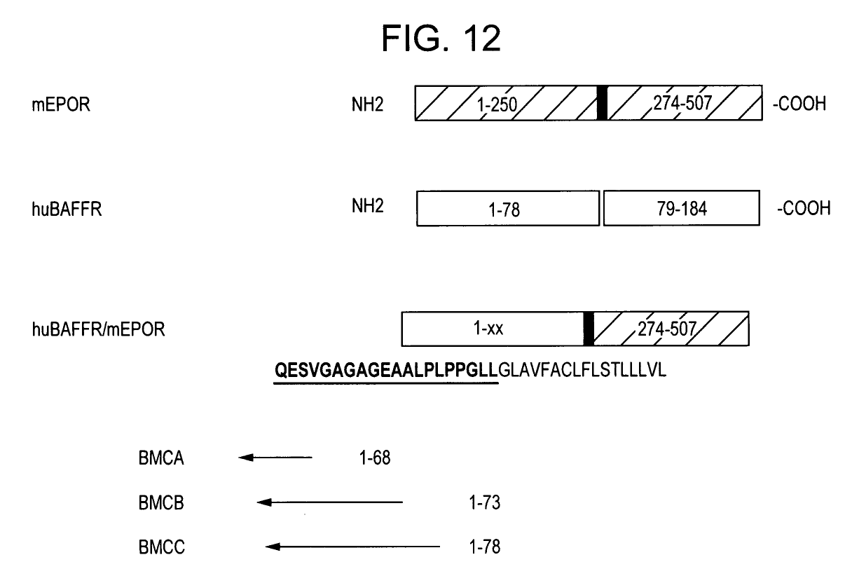

A mouse EPOR and human BAFFR hybrid receptor was constructed as represented in FIG. 12, wherein the EPOR trans-membrane domain is indicated in black. Briefly, three different human BAFFR and mEPOR hybrid receptors (BMCA-C) have been constructed. Extracellular domains of human BAFFR (of three different length: BMCA, 68 amino acids; BMCB, 73 amino acids; BMCC, 78 amino acids) were fused with the mouse EPO receptor comprising the trans-membrane and the intracellular domain. The amino acid sequence representing the junction point of the hybrid receptor is shown in FIG. 12, wherein the trans-membrane domain of the mouse EPOR is italicized and the 3′ end of the human BAFFR extracellular sequence is underlined.

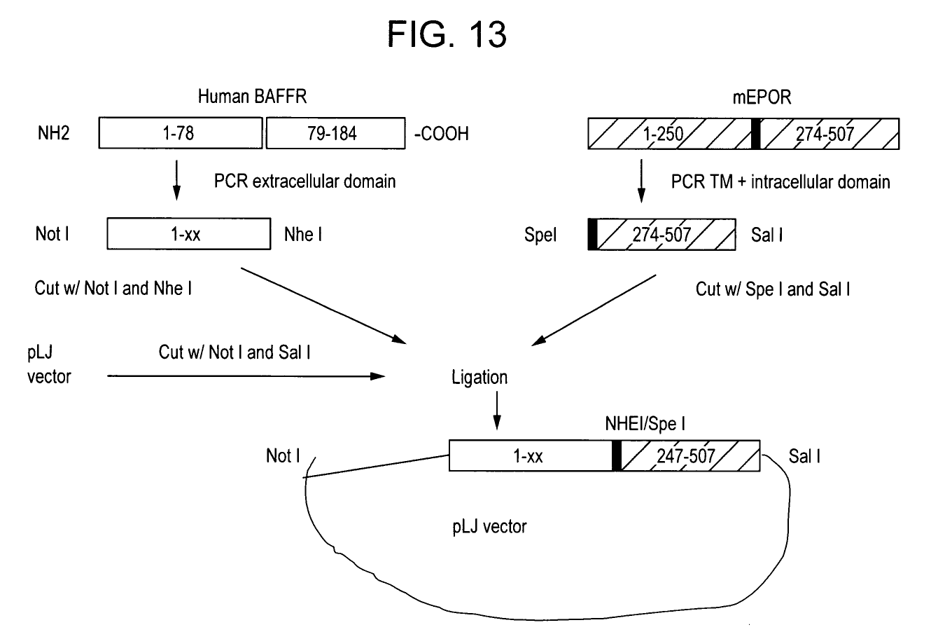

FIG. 13 outlines a generic cloning strategy for making BAFFR/EPOR hybrid receptors. A fragment containing the extracellular domain of human BAFF receptor was obtained by PCR and restriction enzyme digestion by Not I at the 5′ end and Nhe I at the 3′ end. A fragment containing the mouse EPOR trans-membrane and intracellular domain was obtained by PCR and restriction enzyme digestion by Spe I at the 5′ end and Sal at the 3′ end. Two fragments were ligated and subcloned into pLJ vector cut with Not I and Sal I. Positive clones were obtained and sequenced to confirm sequence.

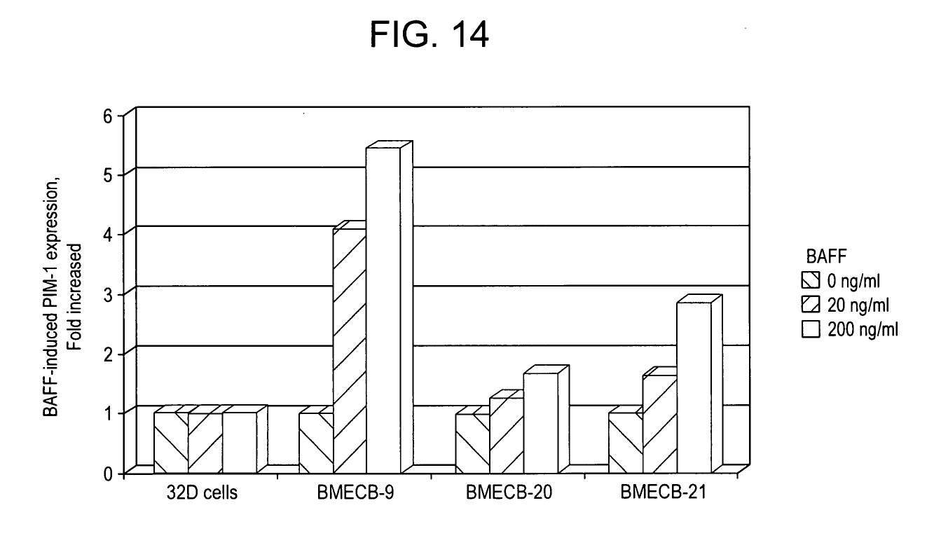

These three different forms of BAFFR/EPOR hybrid receptor construct were transfected into 32Dcl3 cell via electroporation and were selected by medium containing G418 and BAFF to yield 32D/BMC cells. A BAFF responsive cell line BMECB was generated after two rounds of selection and single cell subcloning. These 32D/BMECB cells were maintained in either 5 ng/ml mouse interleukin-3 (mIL-3) or 25 ng/ml of BAFF. BAFF induced PIM-1 expression from 32D/BMECB cells can be measured by bDNA technology. Briefly, three subclones of BMECB cells (BMECB-9, 20, 21) were washed three times, staged overnight with growth factor-free culture medium. Cells were then seeded in 96-well plate in triplicate and treated with BAFF at indicated concentrations at 37° C. for 90 minutes as represented in FIG. 14. Treated cells were lysed and the amount of PIM-1 expression induced by the BAFF treatment was measured by using bDNA QuantiGene kit and illustrated in FIG. 14.

This BAFF-induced expression of PIM-1 in 32D/BMECB cells can be used in biological samples to detect anti-BAFF neutralizing antibody or peptibody, which inhibit the BAFF-induced expression of PIM-1 in 32D/BMECB cells. Similarly, it can be used to detect neutralizing antibodies against the anti-BAFF antibodies or peptibodies, which reverse the inhibitory effects mentioned above. Assays for detecting and measuring the concentrations of neutralizing antibodies against anti-BAFF peptibodies can be performed in 1% human serum, 5% cynomolgus monkey serum, or 2% rat serum samples (Table 5), as no significant matrix effect from these samples was observed.

### EXAMPLE 5

This Example illustrates the validation of a cell-based Neutralizing Antibody (NAb) bioassay for the detection of specific neutralizing activity to a therapeutic protein, such as Osteoprotegerin (OPG) or a monoclonal anti-Receptor Activator of NFκB (RANK) ligand antibody (anti-RANKL) in human serum measuring changes in TRAP (tartrate-resistant acid phosphatase) mRNA using Branched DNA (bDNA) technology (Quantigene, Genospectra, Inc. Fremont, Calif.).

For the determination of neutralizing effects of a human Fc conjugated version of OPG (OPG-Fc) or anti-RANKL, a cell-based bioassay employing a murine macrophage cell line (RAW 264.7), which expresses the receptor for RANK ligand, RANK, was developed. RAW 264.7 cells respond to RANK ligand by differentiating into osteoclast-like cells, expressing the terminal differentiation marker TRAP (tartrate-resistant acid phosphatase). Thus, the inhibition of RANK ligand by OPG-Fc or anti-RANKL would inhibit TRAP mRNA expression as well. However, if neutralizing antibodies to OPG-Fc or anti-RANKL antibodies were present, the TRAP mRNA would continue to be expressed.

Two assays were implements to determine whether there was a specific neutralizing activity to the protein therapeutic, a Screening NAb bioassay and Specificity bioassay. In the Screening NAb Bioassay patient serum was assessed for the presence of neutralizing antibodies to the protein therapeutic while the Specificity bioassay, the protein therapeutic and RANK ligand was used to eliminate false positives due to a non-specific induction of TRAP mRNA.

Example of Bioassay Format for Anti-RANK Ligand Antibody

For the Screening NAb bioassay, a sample containing 5% human patient serum, RANK Ligand and anti-RANKL antibody in cell Growth Media was used. Anti-RANK ligand antibody and RANK Ligand were sequentially added to the serum samples (NAb assay) with incubations for 30 minutes at 37⁰C following each addition RAW 264.7 cells (10,000 cells per well) were added to samples in Screening NAb and Specificity Assays. RAW 264.7 cells were added at 10,000 cells/well and this was incubated for 48 hours at 37° C. TRAP mRNA expression was detected using the Branched DNA Assay below.

For Specificity Bioassay, RAW 264.7 cells were added to a sample containing 5% patient serum only and incubated for 48 hours at 37° C. TRAP mRNA expression was detected using the Branched DNA Assay below.

The following samples were used for controls: Null Control (Control N), 5% pooled human serum; Maximum Control (Control M), 5% pooled human serum and RANK Ligand; Therapeutic Drug Control (Control D), 5% pooled human serum, RANK Ligand and anti-RANKL antibody or OPG; and Positive Control (Control P), 5% pooled human serum, RANK Ligand and anti-RANKL antibody and anti-anti-OPG ligand antibody (500 ng/mL in serum.

Branched DNA Assay. mRNA expression was measured using the Quantigene Screen kit commercially available from Genospectra, Inc. Briefly, RAW264.7 cells were lysed with a buffer containing Label Extender Probes, Blocking Probes and Capture Extender Probes. The cell lysate was transferred to a capture plate and was incubated overnight at 53⁰C. The plate was subsequently washed with a wash buffer (12.5 mL 20X SSC, 7.5 mL 0.01% Lithium Lauryl Sulfate, and 2.48 L water) followed by the addition of bDNA amplifier probe and incubated for 1 hour at 46⁰C. The plate was washed again with wash buffer and bDNA Label Probe was added, and incubated at 46⁰C for 1 hour. The plate was washed for final time with wash buffer followed by the addition of Substrate and a 30 minute incubation at 46⁰C. Luminescence was detected by TopCount NXT reader and measured in Counts per Second (CPS).

TABLE 6

bDNA Probes Used for Murine TRAP mRNA

Capture Extender Probes (CE)

aggagtgggagccatatgattTTTTTctcttggaaagaaagt
tggcgatctctttggcattggTTTTTctcttggaaagaaagt
agttccagcgcttggagatcTTTTTctcttggaaagaaagt
gtccgtgctcggcgatggaTTTTTctcttggaaagaaagt
tctcgtcctgaagatactgcaTTTTTctcttggaaagaaagt
tgaagccgcccagggagtcctcaTTTTTctcttggaaagaaagt
aagagtgattttccagaggcttTTTTTctcttggaaagaaagt

Label Extender Probes (LE)

acccatgaatccatccatcctggTTTTTaggcataggacccgtgtct
tgtaggcccagcagcagcaccTTTTTaggcataggacccgtgtct
gggctgtaccgtgggtcTTTTTaggcataggacccgtgtct
gggctgtaccgtgggtcTTTTTaggcataggacccgtgtct
ccccagtcgcccacagccaTTTTTaggcataggacccgtgtct
ccatttcccgggctgtgtggaTTTTTaggcataggacccgtgtct
tcgctggcatcgtgcactcTTTTTaggcataggacccgtgtct
cggtcagagaacacgtcctcTTTTTaggcataggacccgtgtct
tggtttccagccagcacataccTTTTTaggcataggacccgtgtct
agatggccacagttatgtttgTTTTTaggcataggacccgtgtct
gcatcactgtgtccagcataaTTTTTaggcataggacccgtgtct
aagtcatctgagttgccacacaTTTTTaggcataggacccgtgtct
ctgctgccaactgctttttgaTTTTTaggcataggacccgtgtct
cttgacaaggcagcgcgtggTTTTTaggcataggacccgtgtct
tggctaacaatggtcgcaagttTTTTTaggcataggacccgtgtct
ggttgtggtcatgtccacacaTTTTTaggcataggacccgtgtct
cagcacatagcccacaccgtTTTTTaggcataggacccgtgtct
tgatgtcgcacagagggatccTTTTTaggcataggacccgtgtct

Blocking Probes (BL)

caaatctcagggtgggagtgg
atggggcattggggacccct
cagagacatgatgaagtcagc
cagtgaagtagaaattgtcccc
aaaggtctcctggaacctcttg
aggggatgttgcgaagggca
tacgtggaattttgaagcgca
cattttgggctgctgctgactggca
gccaggacagctgagtgcgg
accaaaacgtagtcctccttgg
ccagatggggtagtggccggcc
ggtaggcagtgaccccgtatg
atgaagttgccggccccact
agccgttggggacctttcgt
gatccatagtgaaaccgcaagt
tggggcttatctccacatgtg

Results were analyzed using pre-determined criteria. Three ratios were used to determine the presence of neutralizing activity. The “NAb ratio” consisting of the mean sample CPS/mean CPS of the Therapeutic Drug Control (Control D) was used to screen for a the presence of any neutralizing activity to anti-RANK ligand antibody. The “Post/Pre” ratio consisting of the mean sample CPS of the post-dose sample/mean sample CPS of the pre-dose was used to determine the development of neutralizing activity between the pre and post-doses. The “Specificity Ratio”, consisting of the mean sample CPS of the Nab assay/mean sample CPS of the Specificity Assay, was used to determine if there were a factors in the serum inducing TRAP mRNA expression. In order for a sample to be considered positive for the presence of neutralizing activity as the result of a neutralizing antibody, a serum sample would be required to be found “Positive” in both the “Nab Ratio” and “Post/Pre Ratio,” and be found to not have non-anti-anti-RANK ligand antibody-specific TRAP gene expression.

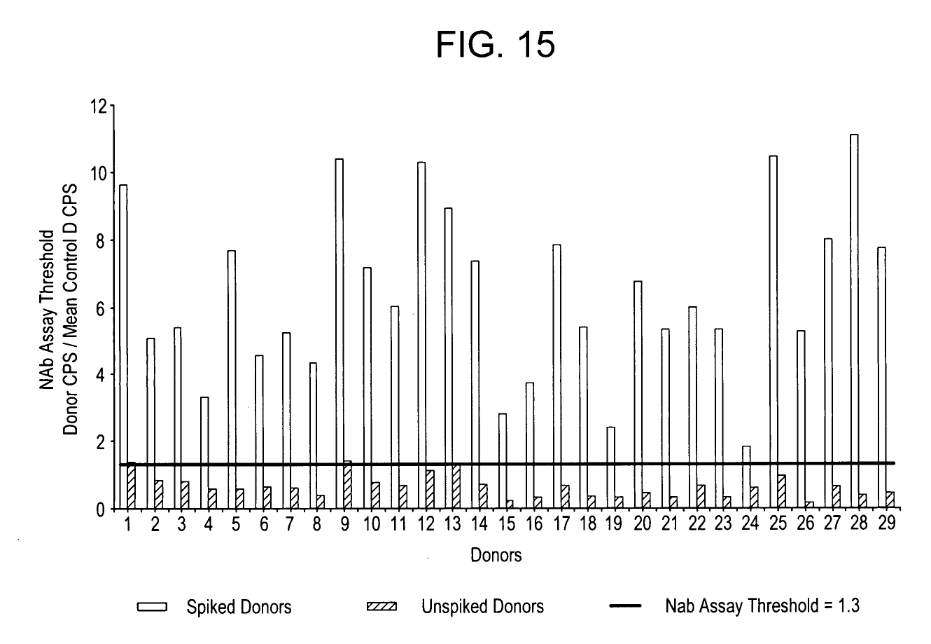

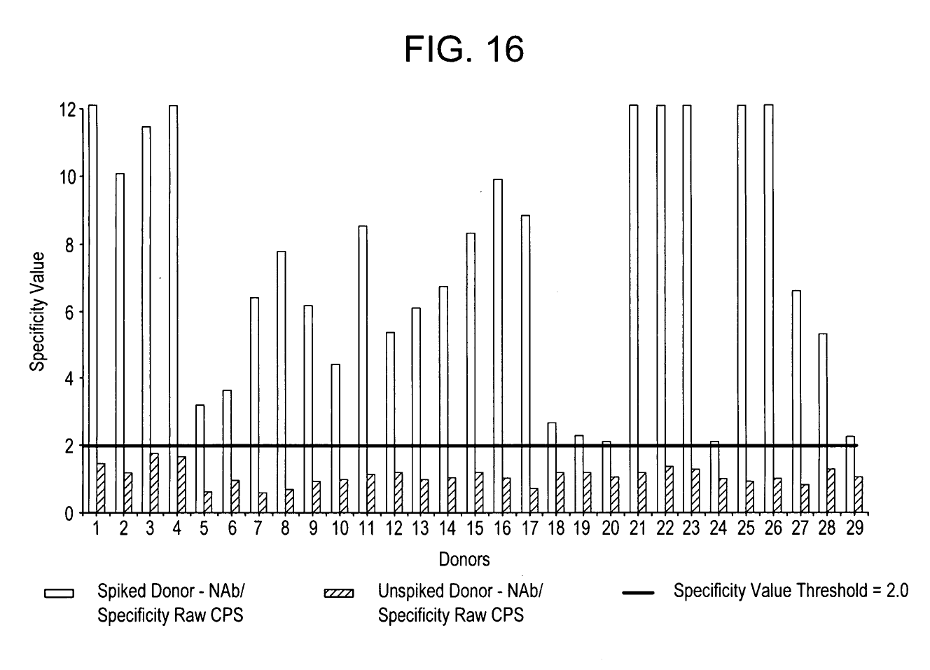

Serum samples from 29 healthy donor volunteers were used to determine donor to donor variability and to derive assay thresholds for both the NAb and Specificity bioassays for the determination of a “positive” and “negative” sample. Results of the cell-based NAb assay are represented in FIG. 15. NAb assay threshold was determined as a ratio of Sample Mean CPS to Control D Mean CPS. Donors were spiked at 500 ng/mL of anti-anti-OPG ligand antibody in neat serum. All spiked donors were found to be above Nab Assay Threshold, and two unspiked donors were found to be above Nab Assay Threshold. FIG. 16 represents Validation of the Specificity Value Threshold. Specificity Value was determined as a ratio of Mean Raw CPS values generated in Nab Assay to Mean Raw CPS Values generated in the Specificity Assay. All spiked donors were found to be above the Specificity Value Threshold, whereas all unspiked donors were found to be below the Specificity Value Threshold, thus, no false negatives or false positives were generated.

TABLE 7

Alignment of mammalian EPO-R amino acid sequences

SEQ

| | |
|---|---|
| ID | Alignment (numbering based on consensus sequence): |

| | | |
|---|---|---|
| | | 1 50 |
| Mouse EPOR | NO:3 | MdklrvplWP rVgpLCLLLA GAaWApsPsl PDpKFESKAA LLAsRGsEEL |
| Rat EPOR | NO:4 | MdqlrvarWP rVspLCLLLA GAaWAssPsl PDpKFESKAA LLAsRGsEEL |
| Human EPOR | NO:2 | MdhlgaslWP qVgsLCLLLA GAaWAppPnl PDpKFESKAA LLAaRGpEEL |
| Pig EPOR | NO:5 | MyhfgatlWP gVgSLCLLLA GAtWApsPns PDaKFESKAA LLAaRGpEEL |
| Consensus | NO:1 | M-------WP -V--LCLLLA GA-WA--P-- PD-KFESKAA LLA-RG-EEL |
| | | 51 100 |
| Mouse EPOR | NO:3 | LCFTqRLEDL VCFWEEAass Gmd.fnYSFS YQLEgEsrKs CsLHQaPTvR |
| Rat EPOR | NO:4 | LCFTqRLEDL VCFWEEAans Gmg.fnYSFS YQLEgEsrKs CrLHQaPTvR |
| Human EPOR | NO:2 | LCFTeRLEDL VCFWEEAasa GVgpgnYSFS YQLEdEpWKl CrLHQaPTaR |
| Pig EPOR | NO:5 | LCFTeRLEDL VCFWEEAgsa GvgpedYSFS YQLEgEpwKp ChLHQgPTaR |
| Consensus | NO:1 | LCFT-RLEDL VCFWEEA--- G-----YSFS YQLE-E--K- C-LHQ-PT-R |
| | | 101 150 |
| Mouse EPOR | NO:3 | GsvRFWCSLP TADTSSFVPL ELqVTe.aSG sPRYHRiIHI NEVVLLDaPa |
| Rat EPOR | NO:4 | GsmRFWCSLP TADTSSFVPL ELqVTe.aSG sPRYHRiIHI NEVVLLDaPa |
| Human EPOR | NO:2 | GavRFWCSLP TADTSSFVPL ELrVT.aaSG aPRYHRvIHI NEVVLLDaPv |
| Pig EPOR | NO:5 | GsvRFWCSLP TADTSSFVPL ELrVTevsSG aPRYHRiIHI NEVVLLDpPa |
| Consensus | NO:1 | G--RFWCSLP TADTSSFVPL EL-VT---SG -PRYHR-IHI NEVVLLD-P- |
| | | 151 200 |
| Mouse EPOR | NO:3 | GLlARrAeEg sHVVLRWLPP PgaPMtthIR YEVdvSagNr AGgtQRVEvL |
| Rat EPOR | NO:4 | GLlARrAeEg sHVVLRWLPP PgaPMtthIR YEVdvSagNr AGgtQRVEvL |
| Human EPOR | NO:2 | GLvARlAdEs gHVVLRWLPP PetPMtshIR YEVdvSagNg AGsvQRVEiL |
| Pig EPOR | NO:5 | GLlARrAeEs gHVVLRWLPP PgaPMaslIR YEVniSteNa AGgvQRVEiL |
| Consensus | NO:1 | GL-AR-A-E- -HVVLRWLPP P--PM---IR YEV--S--N- AG--QRVE-L |
| | | 201 250 |
| Mouse EPOR | NO:3 | eGRTECVLSN LRGgTRYTFa VRARMAEPSF sGFWSAWSEP aSLLTaSDLD |
| Rat EPOR | NO:4 | eGRTECVLSN LRGgTRYTFa VRARMAEPSF sGFWSAWSEP aSLLTaSDLD |
| Human EPOR | NO:2 | eGRTECVLSN LRGrTRYTFa VRARMAEPSF gGFWSAWSEP vSLLTpSDLD |
| Pig EPOR | NO:5 | dGRTECVLSN LRGgTRYTFm VRARMAEPSF gGFWSAWSEP aSLLTaSDLD |
| Consensus | NO:1 | -GRTECVLSN LRG-TRYTF- VRARMAEPSF -GFWSAWSEP -SLLT-SDLD |
| | | 251 300 |
| Mouse EPOR | NO:3 | PLILTLSLIL VlIslLLtVL ALLSHRRtLq QKIWPGIPSP EsEFEGLFTT |
| Rat EPOR | NO:4 | PLILTLSLIL VlIslLLtVL ALLSHRRaLr QKIWPGIPSP EnEFEGLFTT |
| Human EPOR | NO:2 | PLILTLSLIL VvIlvLLtVL ALLSHRRaLk QKIWPGIPSP EsEFEGLFTT |
| Pig EPOR | NO:5 | PLILTLSLIL VlIllLLaVL ALLSHRRtLk QKIWPGIPSP EgEFEGLFTT |
| Consensus | NO:1 | PLILTLSLIL V-I--LL-VL ALLSHRR-L- QKIWPGIPSP E-EFEGLFTT |
| | | 301 350 |
| Mouse EPOR | NO:3 | HKGNFQLWLl QrDGCLWWSP gssFpEDPPA hLEVLSEprW avtQAgdpga |
| Rat EPOR | NO:4 | HKGNFQLWLl QrDGCLWWSP sspFpEDPPA hLEVLSErrW gvtQAgdaga |
| Human EPOR | NO:2 | HKGNFQLWLy QnDGCLWWSP ctpFtEDPPA sLEVLSErcW gtmQAvepgt |
| Pig EPOR | NO:5 | HKGNFQLWLy QtDGCLWWSP ctpFaEDPPA pLEVLSErcW gvtQAvepaa |
| Consensus | NO:1 | HKGNFQLWL- Q-DGCLWWSP ---F-EDPPA -LEVLSE--W ---QA----- |
| | | 351 400 |
| Mouse EPOR | NO:3 | dDeGpLLEPV GSEhAqDTYL VLDkWLLPRt PcSEnLsgPG gsvDpvtMDE |
| Rat EPOR | NO:4 | eDkGpLLEPV GSErAqDTYL VLDeWLLPRc PcSEnLsgPG dsvDpatMDE |
| Human EPOR | NO:2 | dDeGpLLEPV GSEhAqDTYL VLDkWLLPRn PpSEdLpgPG gsvDivaMDE |
| Pig EPOR | NO:5 | dDeGsLLEPV GSEhArDTYL VLDkWLLPRr PaSEdLpqPG gdlDmaaMDE |
| Consensus | NO:1 | -D-G-LLEPV GSE-A-DTYL VLD-WLLPR- P-SE-L--PG ---D---MDE |
| | | 401 450 |
| Mouse EPOR | NO:3 | aSEtSsCpSd LAsKPrPEGt SPsSFEYTIL DPSSqLLcPr aLppELPPTP |
| Rat EPOR | NO:4 | gSEtSsCpSd LAsKPrPEGt SpsSFEYTIL DPSSkLLcPr aLppELPPTP |
| Human EPOR | NO:2 | gSEaSsCsSa LAsKPsPEGa SaaSFEYTIL DPSSqLLrPw tLcpELPPTP |
| Pig EPOR | NO:5 | aSEaSfCsSa LAlKPgPEGa SaaSFEYTIL DPSSqLLrPr aLpaELPPTP |
| Consensus | NO:1 | -SE-S-C-S- LA-KP-PEG- S--SFEYTIL DPSS-LL-P- -L--ELPPTP |
| | | 451 500 |
| Mouse EPOR | NO:3 | PHLKYLYLVV SDSGISTDYS SGgSQgvhGd sSdGPYShPY ENSLvPdsEP |
| Rat EPOR | NO:4 | PHLKYLYLVV SDSGISTDYS SCgSQgvhGd sSdGPYShPY ENSLvPdtEP |
| Human EPOR | NO:2 | PHLKYLYLVV SDSGISTDYS SGdSQgaqGg lSdGPYSnPY ENSLiPaaEP |
| Pig EPOR | NO:5 | PHLKYLYLVV SDSGISTDYS SGgSQetqGg sSsGPYSnPY ENSLvPapEP |
| Consensus | NO:1 | PHLKYLYLVV SDSGISTDYS SG-SQ---G- -S-GPYS-PY ENSL-P--EP |
| | | 501 509 |
| Mouse EPOR | NO:3 | lhPgYVaCS |
| Rat EPOR | NO:4 | lrPsYVaCS |
| Human EPOR | NO:2 | lpPsYVaCS |
| Pig EPOR | NO:5 | spPnYVtCS |
| Consensus | NO:1 | --P-YV-CS |

TABLE 8
Location of domains within EPO-R amino acid sequences; numbering refers
to amino acid positions within the corresponding SEQ ID NOs

| | SEQ | Signal | | | | | “Mature”^{A} |
|---|---|---|---|---|---|---|---|
| Polypeptide | ID | sequence | Extracellular | WSXWS | Transmembrane | Intracellular | polypeptide |
| Mouse EPOR | NO: 3 | 1-24 | 25-249 | 232-236 | 250-272 | 273-507 | 25-507 |
| Rat EPOR | NO: 4 | 1-24 | 25-249 | 232-236 | 250-272 | 273-507 | 25-507 |
| Human EPOR | NO: 2 | 1-24 | 25-250 | 233-237 | 251-273 | 274-508 | 25-508 |
| Pig EPOR | NO: 5 | 1-24 | 25-251 | 234-238 | 252-274 | 275-509 | 25-509 |
| Consensus | NO: 1 | 1-24 | 25-251 | 234-238 | 252-274 | 275-509 | 25-509 |

^{A}For the purposes of the above table, the “mature” polypeptide refers to a polypeptide from which the indicated signal sequence has been cleaved; additional mature polypeptide forms may occur.

TABLE 9

Alignment of mammalian EPO amino acid sequences

SEQ

| | |
|---|---|
| ID | Alignment (numbering based on consensus sequence): |

| | | |
|---|---|---|
| | | 1 50 |
| Mouse EPO | NO:10 | MGvpe.rptl lLlLSllliP LGlPVlcAPp RLiCDSRVLE RYiLeAkEAE |
| Rat EPO | NO:1 | MGvpe.rptl lLlLSllliP LGlPVlcAPp RLiCDSRVLE RYiLeAkEAE |
| Macaque EPO | NO:9 | MGvhecpawl wLlLSlvslP LGlPVpgAPp RLiCDSRVLE RYlLeAkEAE |
| Rhesus EPO | NO:8 | MGvheCpawl wLlLSlVslP LGlPVPgAPp RLvCDSRVLE RYlLeAkEAE |
| Human EPO | NO:7 | MGvhecpawl wLlLSllslP LGlPVlgAPp RLiCDSRVLE RYlLeAkEAE |
| Cow EPO | NO:12 | MGardctp.. lLmLSfllfp LGfPVlgAPa RLiCDSRVLE RYiLeArEAE |
| Sheep EPO | NO:13 | MGardctpll lLlLSfllfP LGlpVlgAPp RLiCDSRVLE RYiLeArEAE |
| Cat EPO | NO:14 | MGscec.pal lLlLSllllP LGlPVlgAPp RLiCDSRVLE RYiLgArEAE |
| Consensus | NO:6 | MG-------- -L-LS----P LG-PV--AP- RL-CDSRVLE RY-L-A-EAE |
| | | 51 100 |
| Mouse EPO | NO:10 | NvTmGCaEgp rlsENITVPD TKVNFYaWKR meVeeQAiEV WQGLsLLSEA |
| Rat EPO | NO:11 | NvTmGCaEgp rlsENITVPD TKVNFYaWKR mkVeeQAvEV WQGLsLLSEA |
| Macaque EPO | NO:9 | NvTmGCsEsc slnENITVPD TKVNFYaWKR meVgqQAvEV WQGLaLLSEA |
| Rhesus EPO | NO:8 | NvTmGCsEsc slnENITVPD TKVNFYaWKR ieVgqQAvEV WQGLaLLSEA |
| Human EPO | NO:7 | NiTtGCaEhc slnENITVPD TKVNFYaWKR meVgqQAvEV WQGLaLLSEA |
| Cow EPO | NO:12 | NaTmGCaEgc sfnENITVPD TKVNFYaWKR meVqqQAlEV WQGLaLLSEA |
| Sheep EPO | NO:13 | NaTmGCaEgc sfsENITVPD TKVNFYaWKR meVqqQAlEV WQGLaLLSEA |
| Cat EPO | NO:14 | NvTmGCaEgc sfsENITVPD TKVNFYtWKR mdVgqQAvEV WQGLaLLSEA |
| Consensus | NO:6 | N-T-GC-E-- ---ENITVPD TKVNFY-WKR --V--QA-EV WQGL-LLSEA |
| | | 101 150 |
| Mouse EPO | NO:10 | ilqaQAllaN sSQPpEtLqL HiDKAiSgLR SlTsLLRvLG AQkElmspPd |
| Rat EPO | NO:11 | ilqaQAlqaN sSQPpEsLqL HiDKAiSgLR SlTsLLRvLG AQkElmspPd |
| Macaque EPO | NO:9 | vlrgQAvlaN sSQPfEpLqL HmDKAiSgLR SiTtLLRaLG AQ.EaislPd |
| Rhesus EPO | NO:8 | vlrgQAvlaN sSQPfEpLqL HmDKAiSgLR SiTtLLRaLG AQ.EaislPd |
| Human EPO | NO:7 | vlrgQAllvN sSQPwEpLqL HvDKAvSgLR SlTtLLRaLG AQkEaispPd |
| Cow EPO | NO:12 | ilrgQAllaN aSQPcEaLrL HvDKAvSgLR SlTsLLRaLG AQkEaislPd |
| Sheep EPO | NO:13 | ifrgQAllaN aSQPcEaLrL HvDKAvSgLR SlTsLLRaLG AQkEaiplPd |
| Cat EPO | NO:14 | ilrgQAllaN sSQPsEtLqL HvDKAvSsLR SlTsLLRaLG AQkEatslPe |
| Consensus | NO:6 | ----QA---N -SQP-E-L-L H-DKA-S-LR S-T-LLR-LG AQ-E----P- |
| | | 151 194 |
| Mouse EPO | NO:10 | ttp.pAPLRt lTvDtfcKLF RvYaNFLRGK LkLYTGEvCR rGDR |
| Rat EPO | NO:11 | atq.aAPLRt lTaDtfcKLF RvYsNFLRGK LkLYTGEaCR rGDR |
| Macague EPO | NO:9 | aa.saAPLRt iTaDtfcKLF RvYsNFLRGK LkLYTGEaCR rGDR |
| Rhesus EPO | NO:8 | aa.saAPLRt iTaDtfcKLF RvYSNFLRGK LkLYTGEaCR rGDR |
| Human EPO | NO:7 | aa.saAPLRt iTaDtfrKLF RvYsNFLRGK LkLYTGEaCR tGDR |
| Cow EPO | NO:12 | atpsaAPLRa fTvDalsKLF RiYsNFLRGK LtLYTGEaCR rGDR |
| Sheep EPO | NO:13 | atpsaAPLRi fTvDalsKLF RiYsNFLRGK LtLYTGEaCR rGDR |
| Cat EPO | NO:14 | at.saAPLRt fTvDtlcKLF RiYsNFLRGK LtLYTGEaCR rGDR |
| Consensus | NO:6 | -----APLR- -T-D---KLF R-Y-NFLRGK L-LYTGE-CR -GDR |

TABLE 10
Location of domains within EPO amino acid sequences; numbering
refers to amino acid positions within the corresponding SEQ ID NOs

| Polypeptide | SEQ ID | Signal sequence | “Mature”^{A} polypeptide |
|---|---|---|---|
| Mouse EPO | NO: 10 | 1-26 | 27-192 |
| Rat EPO | NO: 11 | 1-26 | 27-192 |
| Macaque EPO | NO: 9 | 1-27 | 28-192 |
| Rhesus EPO | NO: 8 | 1-27 | 28-192 |
| Human EPO | NO: 7 | 1-27 | 28-193 |
| Cow EPO | NO: 12 | 1-25 | 26-192 |
| Sheep EPO | NO: 13 | 1-27 | 28-194 |
| Cat EPO | NO: 14 | 1-26 | 27-192 |
| Consensus | NO: 6 | 1-27 | 28-194 |

^{A}For the purposes of the above table, the “mature” polypeptide refers to a polypeptide from which the indicated signal sequence has been cleaved; additional mature polypeptide forms may occur.

TABLE 11

Alignment of mammalian PIM1 coding (cDNA) nucleotide
sequences

SEQ

| | |
|---|---|
| ID | Alignment (numbering based on consensus sequence): |

| | | |
|---|---|---|
| | | 1 50 |
| Mouse PIM1 | NO:21 | ATGCTCcTGT CCAAgATCAA CTCcCTgGCC CACCTGCGCg CcGCgCCcTG |
| Rat PIM1 | NO:20 | ATGCTCtTGT CCAAgATCAA CTCcCTgGCC CACCTGCGCg CaGCcCCtTG |
| Cat PIM1 | NO:18 | ATGCTCtTGT CCAAaATCAA CTCgCTtGCC CACCTGCGCa CcGCgCCcTG |
| Human PIM1 | NO:16 | ATGCTCtTGT CCAAaATCAA CTCgCTtGCC CACCTGCGCg CcGCgCCcTG |
| Cow PIM1 | NO:19 | ATGCTCtTGT CCAAaATCAA CTCgCTtGCC CACCTGCGCg CcGCgCCcTG |
| Chimp PIM1 | NO:17 | ATGCTCtTGT CCAAaATCAA CTCgCTtGCC CACCTGCGCg CcGCgCCcTG |
| Consensus^{A} | NO:15 | ATGCTCYTGT CCAARATCAA CTCSCTKGCC CACCTGCGCR CMGCSCCYTG |
| | | 51 100 |
| Mouse PIM1 | NO:21 | CAaCGACCTG CACGCCAcCA AGCTGGCGCC gGGCAAaGAG AAGGAGCCCC |
| Rat PIM1 | NO:20 | CAaCGACCTG CACGCCAaCA AGCTGGCGCC gGGCAAaGAG AAGGAGCCCC |
| Cat PIM1 | NO:18 | CAaCGACCTG CACGCCAcCA AGCTGGCGCC cGGCAAgGAG AAGGAGCCCC |
| Human PIM1 | NO:16 | CAaCGACCTG CACGCCAcCA AGCTGGCGCC cGGCAAgGAG AAGGAGCCCC |
| Cow PIM1 | NO:19 | CAgCGACCTG CACGCCAcCA AGCTGGCGCC gGGCAAgGAG AAGGAGCCCC |
| Chimp PIM1 | NO:17 | CAaCGACCTG CACGCCAcCA AGCTGGCGCC cGGCAAgGAG AAGGAGCCCC |
| Consensus | NO:15 | CARCGACCTG CACGCCAMCA AGCTGGCGCC SGGCAARGAG AAGGAGCCCC |
| | | 101 150 |
| Mouse PIM1 | NO:21 | TGGAGTCGCA GTACCAGGTG GGCCCGCTgt TGGGCAGcGG tGGCTTCGGC |
| Rat PIM1 | NO:20 | TGGAGTCGCA GTACCAGGTG GGCCCGCTgt TGGGCAGcGG tGGCTTCGGC |
| Cat PIM1 | NO:18 | TGGAGTCGCA GTACCAGGTG GGCCCGCTac TGGGCAGcGG cGGCTTCGGC |
| Human PIM1 | NO:16 | TGGAGTCGCA GTACCAGGTG GGCCCGCTac TGGGCAGcGG cGGCTTCGGC |
| Cow PIM1 | NO:19 | TGGAGTCGCA GTACCAGGTG GGCCCGCTcc TGGGCAGtGG cGGCTTCGGC |
| Chimp PIM1 | NO:17 | TGGAGTCGCA GTACCAGGTG GGCCCGCTac TGGGCAGcGG cGGCTTCGGC |
| Consensus | NO:15 | TGGAGTCGCA GTACCAGGTG GGCCCGCTVY TGGGCAGYGG YGGCTTCGGC |
| | | 151 200 |
| Mouse PIM1 | NO:21 | TCGGTcTACT CtGGCATCCG cGTCgCCGAC AACTTGCCGG TGGCCATtAA |
| Rat PIM1 | NO:20 | TCGGTcTACT CgGGCATCCG cGTCgCCGAC AACTTGCCGG TGGCCATcAA |
| Cat PIM1 | NO:18 | TCGGTcTACT CaGGCATCCG gGTCgCCGAC AACTTGCCGG TGGCCATcAA |
| Human PIM1 | NO:16 | TCGGTcTACT CaGGCATCCG cGTCtCCGAC AACTTGCCGG TGGCCATcAA |
| Cow PIM1 | NO:19 | TCGGTgTACT CaGGCATCCG tGTCgCCGAC AACTTGCCGG TGGCCATcAA |
| Chimp PIM1 | NO:17 | TCGGTcTACT CaGGCATCCG cGTCtCCGAC AACTTGCCGG TGGCCATcAA |
| Consensus | NO:15 | TCGGTSTACT CDGGCATCCG BGTCKCCGAC AACTTGCCGG TGGCCATYAA |
| | | 201 250 |
| Mouse PIM1 | NO:21 | gCACGTGGAG AAGGACCGGA TTTCCGAtTG GGGaGAaCTG CCcAAtGGCA |
| Rat PIM1 | NO:20 | gCACGTGGAG AAGGACCGGA TTTCCGAcTG GGGgGAaCTG CCcAAcGGCA |
| Cat PIM1 | NO:18 | gCACGTGGAG AAGGACCGGA TTTCCGAtTG GGGaGAgCTG CCcAAtGGCA |
| Human PIM1 | NO:16 | aCACGTGGAG AAGGACCGGA TTTCCGAcTG GGGaGAgCTG CCtAAtGGCA |
| Cow PIM1 | NO:19 | gCACGTGGAG AAGGACCGGA TTTCCGAcTG GGGaGAgCTG CCtAAtGGCA |
| Chimp PIM1 | NO:17 | aCACGTGGAG AAGGACCGGA TTTCCGAcTG GGGaGAgCTG CCtAAtGGCA |
| Consensus | NO:15 | RCACGTGGAG AAGGACCGGA TTTCCGAYTG GGGRGARCTG CCYAAYGGCA |
| | | 251 300 |
| Mouse PIM1 | NO:21 | CcCGAGTGCC CATGGAaGTG GTcCTGtTGA AGAAGGTGAG CTCGGacTTC |
| Rat PIM1 | NO:20 | CcCGAGTGCC CATGGAaGTG GTcCTGcTGA AGAAGGTGAG CTCGGgcTTC |
| Cat PIM1 | NO:18 | CcCGAGTGCC CATGGAgGTG GTcCTGcTGA AGAAGGTGAG CTCGGgcTTC |
| Human PIM1 | NO:16 | CtCGAGTGCC CATGGAaGTG GTcCTGcTGA AGAAGGTGAG CTCGGgtTTC |
| Cow PIM1 | NO:19 | CcCGAGTGCC CATGGAaGTG GTtCTGcTGA AGAAGGTGAG CTCGGgcTTC |
| Chimp PIM1 | NO:17 | CtCGAGTGCC CATGGAaGTG GTcCTGcTGA AGAAGGTGAG CTCGGgtTTC |
| Consensus | NO:15 | CYCGAGTGCC CATGGARGTG CTYCTGYTGA AGAAGGTGAG CTCGGRYTTC |
| | | 301 350 |
| Mouse PIM1 | NO:21 | TCgGGCGTCA TTaGaCTtCT GGACTGGTTc GAGAGGCCCG AtAGTTTCGT |
| Rat PIM1 | NO:20 | TCgGGCGTCA TTaGaCTtCT GGACTGGTTc GAGAGGCCCG AtAGTTTCGT |
| Cat PIM1 | NO:18 | TCcGGCGTCA TTcGgCTcCT GGACTGGTTt GAGAGGCCCG AcAGTTTCGT |
| Human PIM1 | NO:16 | TCcGGCGTCA TTaGgCTcCT GGACTGGTTc GAGAGGCCCG AcAGTTTCGT |
| Cow PIM1 | NO:19 | TCcGGCGTCA TTaGgCTcCT GGACTGGTTc GAGAGGCCCG AcAGTTTCGT |
| Chimp PIM1 | NO:17 | TCcGGCGTCA TTaGgCTcCT GGACTGGTTc GAGAGGCCCG AcAGTTTCGT |
| Consensus | NO:15 | TCSGGCGTCA TTMGRCTYCT GGACTGGTTY GAGAGGCCCG AYAGTTTCGT |
| | | 351 400 |
| Mouse PIM1 | NO:21 | gcTGATCCTG GAGAGGCCcG AaCCgGTGCA AGAcCTCTTC GACTTtATCA |
| Rat PIM1 | NO:20 | gcTGATCCTG GAGAGGCCcG AaCCcGTGCA AGAcCTCTTC GACTTcATCA |
| Cat PIM1 | NO:18 | ctTGATCCTG GAGAGGCCcG AgCCgGTGCA AGAcCTCTTC GACTTtATCA |
| Human PIM1 | NO:16 | ccTGATCCTG GAGAGGCCcG AgCCgGTGCA AGAtCTCTTC GACTTcATCA |
| Cow PIM1 | NO:19 | ccTGATCCTG GAGAGGCCgG AgCCgGTGCA AGAcCTCTTC GACTTtATCA |
| Chimp PIM1 | NO:17 | ccTGATCCTG GAGAGGCCcG AgCCgGTGCA AGAtCTCTTC GACTTtATCA |
| Consensus | NO:15 | SYTGATCCTG GAGAGGCCSG ARCCSGTGCA AGAYCTCTTC GACTTYATCA |
| | | 401 450 |
| Mouse PIM1 | NO:21 | CcGAacGaGG aGCcCTaCAg GAGGAcCTgG CCCGagGaTT CTTCTGGCAG |
| Rat PIM1 | NO:20 | CcGAgcGaGG aGCcCTcCAg GAGGAgCTgG CCCGgaGcTT CTTCTGGCAG |
| Cat PIM1 | NO:18 | CgGAaaGgGG gGCtCTgCAg GAGGAgCTgG CCCGcaGcTT CTTCTGGCAG |
| Human PIM1 | NO:16 | CgGAaaGgGG aGCcCTgCAa GAGGAgCTcG CCCGcaGcTT CTTCTGGCAG |
| Cow PIM1 | NO:19 | CgGAaaGgGG gGCtCTgCAg GAGGAgCTgG CCCGcaGcTT CTTCTGGCAG |
| Chimp PIM1 | NO:17 | CgGAaaGgGG gGCcCTgCAa GAGGAgCTgG CCCGcaGcTT CTTCTGGCAG |
| Consensus | NO:15 | CSGARMGRCG RGCYCTVCAR GAGGASCTSG CCCGVRGMTT CTTCTGGCAG |
| | | 451 500 |
| Mouse PIM1 | NO:21 | GTgCTGGAGG CcGTGCGgCA tTGCCACaAC TGCGGGGTtC TcCACCGCGA |
| Rat PIM1 | NO:20 | GTgCTGGAGG CcGTGCGgCA tTGCCACaAC TGCGGGGTtC TcCACCGCGA |
| Cat PIM1 | NO:18 | GTgCTGGAGG CcGTGCGgCA cTGCCACaAC TGCGGGGTgC TcCACCGCGA |
| Human PIM1 | NO:16 | GTgCTGGAGG CcGTGCGgCA cTGCCACaAC TGCGGGGTgC TcCACCGCGA |
| Cow PIM1 | NO:19 | GTaCTGGAGG CgGTGCGaCA cTGCCACgAC TGCGGGGTgC TtCACCGCGA |
| Chimp PIM1 | NO:17 | GTgCTGGAGG CcGTGCGgCA cTGCCACaAC TGCGGGGTgC TcCACCGCGA |
| Consensus | NO:15 | GTRCTGGAGG CSGTGCGRCA YTGCCACRAC TGCGGGGTKC TYCACCGCGA |
| | | 501 550 |
| Mouse PIM1 | NO:21 | CATCAAGGAC GAgAACATCt TaATCGACCT gAgcCGCGGC GAaaTCAAaC |
| Rat PIM1 | NO:20 | CATCAAGGAC GAgAACATCt TaATCGACCT gAacCGCGGC GAacTCAAaC |
| Cat PIM1 | NO:18 | CATCAAGGAC GAgAACATCc TcATCGACCT cAatCGCGGC GAgcTCAAgC |
| Human PIM1 | NO:16 | CATCAAGGAC GAaAACATCc TtATCGACCT cAatCGCGGC GAgcTCAAgC |
| Cow PIM1 | NO:19 | CATCAAGGAC GAgAACATCc TtATCGACCT cAatCGCGGC GAgcTCAAgC |
| Chimp PIM1 | NO:17 | CATCAAGGAC GAaAACATCc TtATCGACCT cAatCGCGGC GAgcTCAAgC |
| Consensus | NO:15 | CATCAAGGAC GARAACATCY THATCGACCT SARYCGCGGC GARMTCAARC |
| | | 551 600 |
| Mouse PIM1 | NO:21 | TCATCGACTT CGGGTCGGGG GCGCTGCTCA AgGACACaGT CTACACGGAC |
| Rat PIM1 | NO:20 | TCATCGACTT CGGGTCGGGG GCGCTGCTCA AgGACACaGT CTACACGGAC |
| Cat PIM1 | NO:18 | TCATCGACTT CGGGTCGGGG GCGCTGCTCA AgGACACcGT CTACACGGAC |
| Human PIM1 | NO:16 | TCATCGACTT CGGGTCGGGG GCGCTGCTCA AaGACACcGT CTACACGGAC |
| Cow PIM1 | NO:19 | TCATCGACTT CGGGTCGGGG GCGCTGCTCA AgGACACcGT CTACACGGAC |
| Chimp PIM1 | NO:17 | TCATCGACTT CGGGTCGGGG GCGCTGCTCA AgGACACcGT CTACACGGAC |
| Consensus | NO:15 | TCATCGACTT CGGGTCGGGG GCGCTGCTCA ARGACACMGT CTACACGGAC |
| | | 601 650 |
| Mouse PIM1 | NO:21 | TTtGAtGGgA CCCGAGTGTA cAGtCCtCCA GAGTGGATtC GCTAcCATCG |
| Rat PIM1 | NO:20 | TTtGAcGGaA CCCGAGTGTA cAGtCCtCCA GAGTGGATtC GCTAcCATCG |
| Cat PIM1 | NO:18 | TTcGAcGGgA CCCGAGTGTA tAGtCCcCCA GAGTGGATcC GCTAcCATCG |
| Human PIM1 | NO:16 | TTcGAtGGgA CCCGAGTGTA tAGcCCtCCA GAGTGGATcC GCTAcCATCG |
| Cow PIM1 | NO:19 | TTcGAtGGgA CCCGAGTGTA tAGtCCtCCA GAGTGGATcC GCTAtCATCG |
| Chimp PIM1 | NO:17 | TTcGAtGGgA CCCGAGTGTA tAGcCCtCCA GAGTGGATcC GCTAcCATCG |
| Consensus | NO:15 | TTYGAYGGRA CCCGAGTGTA YAGYCCYCCA GAGTGGATYC GCTAYCATCG |
| | | 651 700 |
| Mouse PIM1 | NO:21 | CTACCAcGGC AGGTCGGCaG CtGTcTGGTC cCTtGGGATC CTGCTcTATG |
| Rat PIM1 | NO:20 | CTACCAcGGC AGGTCGGCtG CtGTtTGGTC cCTgGGGATC CTGCTcTATG |
| Cat PIM1 | NO:18 | CTACCAtGGC AGGTCGGCcG CcGTcTGGTC tCTgGGGATC CTGCTgTATG |
| Human PIM1 | NO:16 | CTACCAtGGC AGGTCGGCgG CaGTcTGGTC cCTgGGGATC CTGCTgTATG |
| Cow PIM1 | NO:19 | CTACCAtGGC AGGTCGGCaG CcGTcTGGTC tCTgGGGATC CTGCTgTATG |
| Chimp PIM1 | NO:17 | CTACCAtGGC AGGTCGGCgG CaGTcTGGTC cCTgGGGATC CTGCTgTATG |
| Consensus | NO:15 | CTACCAYGGC AGGTCGGCNG CHGTYTGGTC YCTKGGGATC CTGCTSTATG |
| | | 701 750 |
| Mouse PIM1 | NO:21 | AcATGgTctg cGGaGAtaTt ccgtttgagc acgatgaaga gatcaTCaag |
| Rat PIM1 | NO:20 | AcATGgTctg cGGaGAtaTt ccatttgagc acgacgaaga gatcgTCaag |
| Cat PIM1 | NO:18 | AtATGgTctg tGGaGAtaTt ccttttgagc atgatgaaga gatcaTCagg |
| Human PIM1 | NO:16 | AtATGgTgtg tGGaGAtaTt cctttcgagc atgacgaaga gatcaTCagg |
| Cow PIM1 | NO:19 | AcATGgTgtg cGGaGAtaTt ccctttgagc acgatgagga gattgTCagg |
| Chimp PIM1 | NO:17 | AtATGtTggt aGGtGAatTg aatcatctca tcatgctcag tggtgTCtca |
| Consensus | NO:15 | AYATGKTSKK HGGWGAWWTK MMNYWYSWSM WYRWBSWVRR KRKYRTCWVR |
| | | 751 800 |
| Mouse PIM1 | NO:21 | ggccAagtgt tctTcAggca aactgtctct TCaGAgTGTC AgCAcCTtAT |
| Rat PIM1 | NO:20 | ggccAagtgt actTtAggca aagggtctct TCaGAaTGTC AaCAtCTtAT |
| Cat PIM1 | NO:18 | ggccAagttt tctTcAggca gagggtctct TCaGAgTGTC AgCAtCTcAT |
| Human PIM1 | NO:16 | ggccAggttt tctTcAggca gagggtctct TCaGAaTGTC AgCAtCTcAT |
| Cow PIM1 | NO:19 | ggccAagttt tctTcAggca gcgggtctcc TCaGAgTGTC AaCAtCTcAT |
| Chimp PIM1 | NO:17 | tcaaAatctc ttgTcAtcat ccttcctatt TCtGAaTGTC AgCAtCTcAT |
| Consensus | NO:15 | KSMMARKYKY WYKTYAKSMW VMBKSYYWYY TCWGARTGTC ARCAYCTYAT |
| | | 801 850 |
| Mouse PIM1 | NO:21 | TAaATGGTGC cTGtCCcTGA GACCaTCaGA tcGGCCctCC TTtGAAGAAA |
| Rat PIM1 | NO:20 | TAgATGGTGC cTGtCCcTGA GACCaTCgGA ccGGCCctCC TTtGAAGAAA |
| Cat PIM1 | NO:18 | TAgATGGTGC tTGgCCcTGA GACCgTCaGA ccGGCCatCC TTcGAAGAAA |
| Human PIM1 | NO:16 | TAgATGGTGC tTGgCCcTGA GACCaTCaGA taGGCCaaCC TTcGAAGAAA |
| Cow PIM1 | NO:19 | TAgATGGTGC tTGgCCtTGA GACCaTCaGA tcGGCCaaCC TTcGAAGAAA |
| Chimp PIM1 | NO:17 | TAgATGGTGC tTGgCCcTGA GACCaTCaGA taGGCCaaCC TTcGAAGAAA |
| Consensus | NO:15 | TARATGGTGC YTGKCCYTGA GACCRTCRGA YMGGCCMWCC TTYGAAGAAA |
| | | 851 900 |
| Mouse PIM1 | NO:21 | TCCgGAACCA TCCaTGGATG CAgGgtGacC TCCTGCCCCA GGcagCttCt |
| Rat PIM1 | NO:20 | TCCaGAACCA TCCgTGGATG CAgGatGttC TCCTGCCCCA GGccaCcgCc |
| Cat PIM1 | NO:18 | TCCaGAACCA TCCcTGGATG CAaGatGtcC TCCTGCCCCA GGaaaCagCc |
| Human PIM1 | NO:16 | TCCaGAACCA TCCaTGGATG CAaGatGttC TCCTGCCCCA GGaaaCtgCt |
| Cow PIM1 | NO:19 | TCCaGAACCA TCCgTGGATG CAaGacGtcC TCCTGCCCCA GGaaaCtgCt |
| Chimp PIM1 | NO:17 | TCCaGAACCA TCCaTGGATG CAaGatGttC TCCTGCCCCA GGaaaCtgCt |
| Consensus | NO:15 | TCCRGAACCA TCCVTGGATG CARGRYGWYC TCCTGCCCCA GGMMRCHKCY |
| | | 901 942 |
| Mouse PIM1 | NO:21 | GAGATcCAtC TgCACAGtCT GTCaCCgggg tCCAGCAAgT AG |
| Rat PIM1 | NO:20 | GAGATtCAtC TgCACAGcCT GTCaCCatca cCCAGCAAaT AG |
| Cat PIM1 | NO:18 | GAGATCCAtC TgCACAGcCT GTCaCCaggg cCCAGCAAaT AG |
| Human PIM1 | NO:16 | GAGATcCAcC TcCACAGcCT GTCgCCgggg cCCAGCAAaT AG |
| Cow PIM1 | NO:19 | GAGATcCAtC TcCACAGcCT GTCgCCaggg cCCAGCAAaT AG |
| Chimp PIM1 | NO:17 | GAGATcCAcC TcCACAGcCT GTCgCCgggg cCCAGCAAaT AG |
| Consensus | NO:15 | GAGATYCAYC TSCACAGYCT GTCRCCRKSR YCCAGCAART AG |

^{A}The symbols in the Consensus sequence conform to Annex C, Appendix 2, TABLE 1 of the Patent Cooperation Treaty Administrative Instructions.

TABLE 12

Alignment of mammalian NGF-R amino acid
sequences

SEQ

| | |
|---|---|
| ID: | Alignment (numbering based on consensus sequence): |

| | | |
|---|---|---|
| | | 1 50 |
| Mouse NGFR | NO:82 | MLRGqRlGQL GWHrpAAGlG sLmtsLmLAc AsAAsCrevC CPvGpSGLRC |
| Rat NGFR | NO:83 | MLRGqRhGQL GWHrpAAGlG gLvtsLmLAc AcAAsCretC CPvGpSGLRC |
| Human NGFR | NO:81 | MLRGgRrGQL GWHswAAGpG sLlawLiLAs AgAApCpdaC CPhGsSGLRC |
| Consensus | NO:80 | MLRG-R-GQL GWH--AAG-G -L---L-LA- A-AA-C---C CP-G-SGLRC |
| | | 51 100 |
| Mouse NGFR | NO:82 | TRaGsLdtLr gLrGAgNLTE LYvENQqhLQ rLEfeDLqGL GELRsLTIVK |
| Rat NGFR | NO:83 | TRaGtLntLr gLrGAgNLTE LYvENQrdLQ rLEfeDLqGL GELRsLTIVK |
| Human NGFR | NO:81 | TRdGaLdsLh hLpGAeNLTE LYiENQqhLQ hLElrDLrGL GELRnLTIVK |
| Consensus | NO:80 | TR-G-L--L- -L-GA-NLTE LY-ENQ--LQ -LE--DL-GL GELR-LTIVK |
| | | 101 150 |
| Mouse NGFR | NO:82 | SGLRFVAPDA FrFTPRLShL NLSsNALESL SWKTVQGLSL QdLtLSGNPL |
| Rat NGFR | NO:83 | SGLRFVAPDA FhFTPRLShL NLSsNALESL SWKTVQGLSL QdLtLSGNPL |
| Human NGFR | NO:81 | SGLRFVAPDA FhFTPRLSrL NLSfNALESL SWKTVQGLSL QeLvLSGNPL |
| Consensus | NO:80 | SGLRFVAPDA F-FTPRLS-L NLS-NALESL SWKTVQGLSL Q-L-LSGNPL |
| | | 151 200 |
| Mouse NGFR | NO:82 | HCSCALfWLQ RWEqEgLcGV htQtLhdsGp GdqflPLgH. .NtSCGVPtv |
| Rat NGFR | NO:83 | HCSCALlWLQ RWEqEdLcGV ytQkLqgsGs GdqflPLgH. .NnSCGVPsv |
| Human NGFR | NO:81 | HCSCALrWLQ RWEeEgLgGV peQkLqchGq G....PLaHm pNaSCGVPtl |
| Consensus | NO:80 | HCSCAL-WLQ RWE-E-L-GV --Q-L---G- G----PL-H- -N-SCGVP-- |
| | | 201 250 |
| Mouse NGFR | NO:82 | KiQmPNdSVe VGDDVfLqCQ VEGlaLqQAd WILTELEgaA TvkKfGdLPS |
| Rat NGFR | NO:83 | KiQmPNdSVe VGDDVfLgCQ VEGqaLqQAd WILTELEgtA TmkKsGdLPS |
| Human NGFR | NO:81 | KvQvPNaSVd VGDDVlLrCQ VEGrgLeQAg WILTELEqsA TvmKsGgLPS |
| Consensus | NO:80 | K-Q-PN-SV- VGDDV-L-CQ VEG--L-QA- WILTELE--A T--K-C-LPS |
| | | 251 300 |
| Mouse NGFR | NO:82 | LGLiLvNVTS DLNkKNvTCW AENDVGRAEV SVQVsVSFPA SVhLglAVEq |
| Rat NGFR | NO:83 | LGLtLvNVTS DLNkKNvTCW AENDVGRAEV SVQVsVSFPA SVhLgkAVEq |
| Human NGFR | NO:81 | LGLtLaNVTS DLNrKNlTCW AENDVGRAEV SVQVnVSFPA SVqLhtAVEm |
| Consensus | NO:80 | LGL-L-NVTS DLN-KN-TCW AENDVGRAEV SVQV-VSFPA SV-L--AVE- |
| | | 301 350 |
| Mouse NGFR | NO:82 | HHWCIPFSVD GQPAPSLRWl FNGSVLNETS FIFTqFLEsA ltNETmRHGC |
| Rat NGFR | NO:83 | HHWCIPFSVD GQPAPSLRWf FNGSVLNETS FIFTqFLEsA ltNETmRHGC |
| Human NGFR | NO:81 | HHWCIPFSVD GQPAPSLRWl FNGSVLNETS FIFTeFLEpA .aNETvRHGC |
| Consensus | NO:80 | HHWCIPFSVD GQPAPSLRW- FNGSVLNETS FIFT-FLE-A --NET-RHGC |
| | | 351 400 |
| Mouse NGFR | NO:82 | LRLNQPTHVN NGNYTLLAAN PyGQAaASvM AAFMDNPFEF NPEDPIPVSF |
| Rat NGFR | NO:83 | LRLNQPTHVN NGNYTLLAAN PyGQAaASiM AAFMDNPFEF NPEDPIPVSF |
| Human NGFR | NO:81 | LRLNQPTHVN NGNYTLLAAN PfGQAsASiM AAFMDNPFEF NPEDPIPVSF |
| Consensus | NO:80 | LRLNQPTHVN NGNYTLLAAN P-GQA-AS-M AAFMDNPFEF NPEDPIPVSF |
| | | 401 450 |
| Mouse NGFR | NO:82 | SPVDgNSTSr DPVEKKDETP FGVSVAVGLA VsAaLFLSaL LLVLNKCGqR |
| Rat NGFR | NO:83 | SPVDtNSTSr DPVEKKDETP FGVSVAVGLA VsAaLFLSaL LLVLNKCGqR |
| Human NGFR | NO:81 | SPVDtNSTSg DPVEKKDETP FGVSVAVGLA VfAcLFLStL LLVLNKCGrR |
| Consensus | NO:80 | SPVD-NSTS- DPVEKKDETP FGVSVAVGLA V-A-LFLS-L LLVLNKCG-R |
| | | 451 500 |
| Mouse NGFR | NO:82 | sKFGINRPAV LAPEDGLAMS LHFMTLGGSS LSPTEGKGSG LQGHImENPQ |
| Rat NGFR | NO:83 | sKFGINRPAV LAPEDGLAMS LHFMTLGGSS LSPTEGKGSG LQGHImENPQ |
| Human NGFR | NO:81 | nKFGINRPAV LAPEDGLAMS LHFMTLGGSS LSPTEGKGSG LQGHIiENPQ |
| Consensus | NO:80 | -KFGINRPAV LAPEDGLAMS LHFMTLGGSS LSPTEGKGSG LQGHI-ENPQ |
| | | 501 550 |
| Mouse NGFR | NO:82 | YFSDtCVHHI KRqDIiLKWE LGEGAFGKVF LAECyNLLnd QDKMLVAVKA |
| Rat NGFR | NO:83 | YFSDtCVHHI KRqDIiLKWE LGEGAFGKVF LAECyNLLnd QDKMLVAVKA |
| Human NGFR | NO:81 | YFSDaCVHHI KRrDIvLKWE LGEGAFGKVF LAEChNLLpe QDKMLVAVKA |
| Consensus | NO:80 | YFSD-CVHHI KR-DI-LKWE LGEGAFGKVF LAEC-NLL-- QDKMLVAVKA |
| | | 551 600 |
| Mouse NGFR | NO:82 | LKEaSEnARQ DFqREAELLT MLQHQHIVRF FGVCTEGgPL LMVFEYMRHG |
| Rat NGFR | NO:83 | LKEtSEnARQ DFhREAELLT MLQHQHIVRF FGVCTEGgPL LMVFEYMRHG |
| Human NGFR | NO:81 | LKEaSEsARQ DFqREAELLT MLQHQHIVRF FGVCTEGrPL LMVFEYMRHG |
| Consensus | NO:80 | LKE-SE-ARQ DF-REAELLT MLQHQHIVRF FGVCTEG-PL LMVFEYMRHG |
| | | 601 650 |
| Mouse NGFR | NO:82 | DLNRFLRSHG PDAKLLAGGE DVAPGPLGLG QLLAVASQVA AGMVYLAsLH |
| Rat NGFR | NO:83 | DLNRFLRSHG PDAKLLAGGE DVAPGPLGLG QLLAVASQVA AGMVYLAsLH |
| Human NGFR | NO:81 | DLNRFLRSHG PDAKLLAGGE DVAPGPLGLG QLLAVASQVA AGMVYLAgLH |
| Consensus | NO:80 | DLNRFLRSHG PDAKLLAGGE DVAPGPLGLG QLLAVASQVA AGMVYLA-LH |
| | | 651 700 |
| Mouse NGFR | NO:82 | FVHRDLATRN CLVGQGLVVK IGDFGMSRDI YSTDYYRVGG RTMLPIRWMP |
| Rat NGFR | NO:83 | FVHRDLATRN CLVGQGLVVK IGDFGMSRDI YSTDYYRVGG RTMLPIRWMP |
| Human NGFR | NO:81 | FVHRDLATRN CLVGQGLVVK IGDFGMSRDI YSTDYYRVGG RTMLPIRWMP |
| Consensus | NO:80 | FVHRDLATRN CLVGQGLVVK IGDFGMSRDI YSTDYYRVGG RTMLPIRWMP |
| | | 701 750 |
| Mouse NGFR | NO:82 | PESILYRKFs TESDVWSFGV VLWEIFTYGK QPWYQLSNTE AIeCITQGRE |
| Rat NGFR | NO:83 | PESILYRKFs TESDVWSFGV VLWEIFTYGK QPWYQLSNTE AIeCITQGRE |
| Human NGFR | NO:81 | PESILYRKFt TESDVWSFGV VLWEIFTYGK QPWYQLSNTE AIdCITQGRE |
| Consensus | NO:80 | PESILYRKF- TESDVWSFGV VLWEIFTYGK QPWYQLSNTE AI-CITQGRE |
| | | 751 801 |
| Mouse NGFR | NO:82 | LERPRACPPd VYAIMRGCWQ REPQQRlSmK DVHARLQALA QAPPsYLDVLG |
| Rat NGFR | NO:83 | LERPRACPPd VYAIMRGCWQ REPQQRlSmK DVHARLQALA QAPPsYLDVLG |
| Human NGFR | NO:81 | LERPRACPPe VYAIMRGCWQ REPQQRhSiK DVHARLQALA QAPPvYLDVLG |
| Consensus | NO:80 | LERPRACPP- VYAIMRGCWQ REPQQR-S-K DVHARLQALA QAPP-YLDVLG |

TABLE 13
Location of domains within NGF-R amino acid sequences; numbering refers
to amino acid positions within the corresponding SEQ ID NOs

| | SEQ | Signal | | | | “Mature”^{A} |
|---|---|---|---|---|---|---|
| Polypeptide | ID | sequence | Extracellular | Transmembrane | Intracellular | polypeptide |
| Mouse NGFR | NO: 82 | 1-32 | 33-418 | 419-442 | 443-799 | 33-799 |
| Rat NGFR | NO: 83 | 1-32 | 33-418 | 419-442 | 443-799 | 33-799 |
| Human NGFR | NO: 81 | 1-32 | 33-415 | 416-439^{B} | 440-796 | 33-796 |
| Consensus | NO: 80 | 1-32 | 33-420 | 421-444 | 445-801 | 33-801 |

^{A}For the purposes of the above table, the “mature” polypeptide refers to a polypeptide from which the indicated signal sequence has been cleaved; additional mature polypeptide forms may occur.
^{B}In the table above, the location of the transmembrane domain in the human NGF-R amino acid sequence is shown as corresponding to the location of the transmembrane domain in the other mammalian NGF-R sequences.

TABLE 14

Alignment of mammalian NGF amino acid sequences

SEQ

| | |
|---|---|
| ID | Alignment (numbering based on consensus sequence): |

| | | |
|---|---|---|
| | | 1 50 |
| Gorilla NGF | NO:86 | MSMLFYTLIT afLIGiQAEl hseSNvpaGh tiPqaHWTKL QHSLDTALRR |
| Orang. NGF | NO:87 | MSMLFYTLIT afLIGiQAEp hseSNvpaGh tiPqaHWTKL QHSLDTALRR |
| Human NGF | NO:85 | MSMLFYTLIT afLIGiQAEp hseSNvpaGh tiPqvHWTKL QHSLDTALRR |
| Mouse NGF | NO:89 | MSMLFYTLIT afLIGvQAEp ytdSNvpeGd svPeaHWTKL QHSLDTALRR |
| Rat NGF | NO:88 | MSMLFYTLIT afLIGvQAEp ytdSNvpeGd svPeaHWTKL QHSLDTALRR |
| Aft. rat NGF | NO:91 | MSMLFYTLIT alLIGvQAEp ytdSNlpeGd svPeaHWTKL QHSLDTALRR |
| G. pig NGF | NO:90 | MSMLFYTLIT vfLIGiQAEp ysdSNvlsGd tiPqaHWTKL QHSLDTALRR |
| Consensus | NO:84 | MSMLFYTLIT --LIG-QAE- ---SN---G- --P--HWTKL QHSLDTALRR |
| | | 51 100 |
| Gorilla NGF | NO:86 | ArSaPaaaIA ARVaGQTrNI TVDPrLFKKR rLrSPRVLFS TQPPpeaaDt |
| Orang. NGF | NO:87 | ArStPaaaIA ARVaGQTcNI TVDPrLFKKR rLrSPRVLFS TQPPpeaaDt |
| Human NGF | NO:85 | ArSaPaaaIA ARVaGQTrNI TVDPrLFKKR rLrSPRVLFS TQPPreaaDt |
| Mouse NGF | NO:89 | ArSaPtapIA ARVtGQTrNI TVDPrLFKKR rLhSPRVLFS TQPPptssDt |
| Rat NGF | NO:88 | ArSaPaepIA ARVtGQTrNI TVDPkLFKKR rLrSPRVLFS TQPPptssDt |
| Afr. rat NGF | NO:91 | ArSaPaapIA ARVtGQTrNI TVDPrLFKKR kLrSPRVLFS TQPPptssDt |
| G. pig NGF | NO:90 | AhSaPaapIA ARVaGQTlNI TVDPrLFKKR rLhSPRVLFS TQPPplstDa |
| Consensus | NO:84 | A-S-P---IA ARV-GQT-NI TVDP-LFKKR -L-SPRVLFS TQPP- - - |
| | | 101 150 |
| Gorilla NGF | NO:86 | qDLDFevgGa apfNRTHRSK RSSsHPiFhr GEFSVCDSVS VWVgDKTTAT |
| Orang. NGF | NO:87 | qDLDFevgGa apfNRTHRSK RSSsHPiFhr GEFSVCDSVS VWVgDKTTAT |
| Human NGF | NO:85 | qDLDFevgGa apfNRTHRSK RSSsHPiFhr GEFSVCDSVS VWVgDKTTAT |
| Mouse NGF | NO:89 | lDLDFqahGt ipfNRTHRSK RSStHPvFhm GEFSVCDSVS VWVgDKTTAT |
| Rat NGF | NO:88 | lDLDFqahGt isfNRTHRSK RSStHPvFhm GEFSVCDSVS VWVgDKTTAT |
| Afr. rat NGF | NO:91 | lDLDFqahGt isfNRTHRSK RSStHPvFqm GEFSVCDSVS VWVgDKTTAT |
| G. pig NGF | NO:90 | qDLDFevdGa asvNRTHRSK RSStHPvFhm GEFSVCDSVS VWVaDKTTAT |
| Consensus | NO:84 | -DLDF---G- ---NRTHRSK RSS-HP-F-- GEFSVCDSVS VWV-DKTTAT |
| | | 151 200 |
| Gorilla NGF | NO:86 | DIKGkEVmVL gEVNiNNsVF kQYFFETKCR dpnPVdSGCR GIDSKHWNSY |
| Orang. NGF | NO:87 | DIKGkEVmVL gEVNiNNsVF kQYFFETKCR dpnPVdSGCR GIDSKHWNSY |
| Human NGF | NO:85 | DIKGkEVmVL gEVNiNNsVF kQYFFETKCR dpnPVdSGCR GIDSKHWNSY |
| Mouse NGF | NO:89 | DIKGkEVlVL aEVNiNNsVF rQYFFETKCR asnPVeSGCR GIDSKHWNSY |
| Rat NGF | NO:88 | DIKGkEVtVL gEVNiNNsVF kQYFFETKCR apnPVeSGCR GIDSKHWNSY |
| Aft. rat NGF | NO:91 | DIKGnEVlVL gEVNiNNsVF kQYFFETKCR arnPVeSGCR GIDSKHWNSY |
| G. pig NGF | NO:90 | DIKGkEVtVL aEVNvNNnVF kQYFFETKCR dpsPVdSGCR GIDSKHWNSY |
| Consensus | NO:84 | DIKG-EV-VL -EVN-NN-VF -QYFFETKCR ---PV-SGCR GIDSKHWNSY |
| | | 201 241 |
| Gorilla NGF | NO:86 | CTTTHTFVKA LTmdgkQAAW RFIRIDTACV CVLsRKAvRR a |
| Orang. NGF | NO:87 | CTTTHTFVKA LTmdgkQAAW RFIRIDTACV CVLsRKAvRR a |
| Human NGF | NO:85 | CTTTHTFVKA LTmdgkQAAW RFIRIDTACV CVLsRKAvRR a |
| Mouse NGF | NO:89 | CTTTHTFVKA LTtdekQAAW RFIRIDTACV CVLsRKAtRR g |
| Rat NGF | NO:88 | CTTTHTFVKA LTtddkQAAW RFIRIDTACV CVLsRKAaRR g |
| Aft. rat NGF | NO:91 | CTTTHTFVKA LTtddrQAAW RFIRIDTACV CVLtRKApRR g |
| G. pig NGF | NO:90 | CTTTHTFVKA LTtankQAAW RFIRIDTACV CVLnRKAaRR g |
| Consensus | NO:84 | CTTTHTFVKA LT----QAAW RFIRIDTACV CVL-RKA-RR - |

TABLE 15
Location of domains within NGF amino acid sequences; numbering
refers to amino acid positions within the corresponding SEQ ID NOs

| | | Signal | | “Mature”^{A} |
|---|---|---|---|---|
| Polypeptide | SEQ ID | sequence | Pro-peptide | polypeptide |
| Gorilla NGF^{B} | NO: 86 | 1-18 | 19-121 | 122-241 |
| Orang. NGF^{B} | NO: 87 | 1-18 | 19-121 | 122-241 |
| Human NGF | NO: 85 | 1-18 | 19-121 | 122-241 |
| Mouse NGF | NO: 89 | 1-18 | 19-121 | 122-241 |
| Rat NGF | NO: 88 | 1-18 | 19-121 | 122-241 |
| Afr. rat NGF | NO: 91 | 1-18 | 19-121 | 122-241 |
| G. pig NGF | NO: 90 | 1-18 | 19-121 | 122-241 |
| Consensus | NO: 84 | 1-18 | 19-121 | 122-241 |

^{A}For the purposes of the above table, the “mature” polypeptide refers to a polypeptide from which the indicated signal sequence and pro-peptide have been cleaved; additional mature polypeptide forms may occur.
^{B}The location of the signal sequence, pro-peptide domain, and the mature polypeptide within the gorilla and orangutan NGF amino acid sequences was based on the location of these domains in the amino acid sequences of the other mammalian NGF amino acid sequences.

TABLE 16

Alignment of mammalian BAFF-R amino acid sequences

SEQ

| | |
|---|---|
| ID: | Alignment (numbering based on consensus sequence): |

| | | |
|---|---|---|
| | | 1 50 |
| Human BAFFR | NO 93 | ˜˜˜mRrgpRS lRgRDapaPT pCvpaECFDl LVRhCVaCgL lrTprPkpag |
| Mouse BAFFR | NO:94 | mgarRlrvRS qRsRDssvPT qCnqtECFDp LVRnCVsCeL fhT..Pdtgh |
| Consensus | NO:92 | ----R---RS -R-RD---PT -C---ECFD- LVR-CV-C-L --T--P--- |
| | | 51 100 |
| Human BAFFR | NO:93 | aSSpaPrTAL QPQEsvgaga GeAalPlpgL LfGAPALLGL aLvLaLV.LV |
| Mouse BAFFR | NO:94 | tSSlePgTAL QPQE...... GsAlrPdvaL LvGAPALLGL iLaLtLVgLV |
| Consensus | NO:92 | -SS--P-TAL QPQE------ G-A--P---L L-GAPALLGL -L-L-LV-LV |
| | | 101 150 |
| Human BAFFR | NO:93 | gLVSWRrRQr rLRgASsaea PDgdkda.pE pLdkViilSp gisdAtAPaW |
| Mouse BAFFR | NO:94 | sLVSWRwRQ. qLRtAS.... PDtsegvqqE sLenVfvpSs etphAsAPtW |
| Consensus | NO:92 | -LVSWR-RQ- -LR-AS---- PD-------E -L--V---S- ----A-AP-W |
| | | 151 189 |
| Human BAFFR | NO:93 | PPpgEDpgtt pPgHSVPVPA TELGSTELVT TKTAGPEQq |
| Mouse BAFFR | NO:94 | PPlkEDadsa lPrHSVPVPA TELGSTELVT TKTAGPEQ˜ |
| Consensus | NO:92 | PP--ED---- -P-HSVPVPA TELGSTELVT TKTAGPEQ- |

TABLE 17
Location of domains within BAFF-R amino acid sequences; numbering refers
to amino acid positions within the corresponding SEQ ID NOs

| | SEQ | | | | |
|---|---|---|---|---|---|
| Polypeptide | ID | Extracellular | Disulfide bonds | Transmembrane | Intracellular |
| Human BAFFR | NO: 93 | 1-76 | (19, 32); (24, 35) | 77-96 | 97-184 |
| Mouse BAFFR | NO: 94 | 1-71 | (22, 35); (27, 38) | 72-92^{A} | 93-175 |
| Consensus | NO: 92 | 1-79 | (22, 35); (27, 38) | 80-100 | 101-189 |

^{A}The location of the transmembrane domain in the human BAFF-R amino acid sequence is shown as corresponding to the location of the transmembrane domain in the mouse BAFF-R sequence.

TABLE 18

Alignment of mammalian BAFF amino acid sequences

SEQ

| | |
|---|---|
| ID: | Alignment (numbering based on consensus sequence): |

| | | |
|---|---|---|
| | | 1 50 |
| Human BAFF | NO:96 | MDdSter.eq srLtsClkKr EeMKlkecvs IlPrKEsps. vrsskDGkLL |
| Mouse BAFF | NO:97 | MDeSaktlpp pcLcfCseKg EdMKv.gydp ItPqKEegaw fgicrDGrLL |
| Consensus | NO:95 | MD-S------ --L--C--K- E-MK------ I-P-KE---- -----DG-LL |
| | | 51 100 |
| Human BAFF | NO:96 | AATLLLALLS cclTvvSfYQ vAALQgDLas LRaELQghha eklPAgAGAP |
| Mouse BAFF | NO:97 | AATLLLALLS ssfTamSlYQ lAALQaDLmn LRmELQsyrg satPAaAGAP |
| Consensus | NO:95 | AATLLLALLS ---T--S-YQ -AALQ-DL-- LR-ELQ---- ---PA-AGAP |
| | | 101 150 |
| Human BAFF | NO:96 | Kagleeapav TAGlKifePp APgegNSSqn sRNkRAvQGP EET....... |
| Mouse BAFF | NO:97 | e........l TAGvKlltPa APrphNSSrg hRNrRAfQGP EETeqdvdls |
| Consensus | NO:95 | ---------- TAG-K---P- AP---NSS-- -RN-RA-QGP EET------- |
| | | 151 200 |
| Human BAFF | NO:96 | .......... .......... ....vtQDCL QLIADSeTPT IqKGsYTFVP |
| Mouse BAFF | NO:97 | Appapclpgc rhsqhddngm nlrniiQDCL QLIADSdTPT IrKGtYPFVP |
| Consensus | NO:95 | ---------- ---------- ------QDCL QLIADS-TPT I-KG-YTFVP |
| | | 201 250 |
| Human BAFF | NO:96 | WLLSFKRGsA LEEKENKIlV keTGYFFIYg QVLYTDktyA MGHlIQRKKV |
| Mouse BAFF | NO:97 | WLLSFKRGnA LEEKENKIvV rqTGYFFIYs QVLYTDpifA MGHvIQRKKV |
| Consensus | NO:95 | WLLSFKRG-A LEEKENKI-V --TGYFFIY- QVLYTD---A MGH-IQRKRV |
| | | 251 300 |
| Human BAFF | NO:96 | HVFGDELSLV TLFRCIQNMP eTLPNNSCYS AGIAkLEEGD ElQLAIPREN |
| Mouse BAFF | NO:97 | HVFGDELSLV TLFRCIQNMP kTLPNNSCYS AGIArLEEGD EiQLAIPREN |
| Consensus | NO:95 | HVFGDELSLV TLFRCIQNMP -TLPNNSCYS AGIA-LEEGD E-QLAIPREN |
| | | 301 318 |
| Human BAFF | NO:96 | AQISldGDvT FFGALKLL |
| Mouse BAFF | NO:97 | AQISrnGDdT FFGALKLL |
| Consensus | NO:95 | AQIS--GD-T FFGALKLL |

TABLE 19
Location of domains within BAFF amino acid sequences; numbering refers to
amino acid positions within the corresponding SEQ ID NOs

| Polypeptide | SEQ ID | Intracellular | Transmembrane | Extracellular | Mature (soluble)^{A} |
|---|---|---|---|---|---|
| Human BAFF | NO: 96 | 1-46 | 47-67 | 68-285 | 134-285 |
| Mouse BAFF | NO: 97 | 1-47 | 48-68 | 69-309 | 127-309 |
| Consensus | NO: 95 | 1-48 | 49-69 | 70-318 | 136-318 |

^{A}For the purposes of the above table, the “mature” polypeptide refers to an extracellular domain of the polypeptide which has been cleaved from the cell surface to form a soluble polypeptide; other mature forms may occur.

TABLE 20

Alignment of mammalian TNFR1 amino acid sequences

SEQ

| | |
|---|---|
| ID: | Alignment (numbering based on consensus sequence): |

| | | |
|---|---|---|
| Mouse | NO:103 | MGLptVPgLL lsLVLlaLLm gihPsgVtGL VpslgDREKR dslCPQGKYv |
| TNFR1 | | |
| Rat | NO:102 | MGLpiVPgLL lsLVLlaLLm gihPsgVtGL VpslgDREKR dnlCPQGKYa |
| TNFR1 | | |
| Cat | NO:100 | MGLptVPgLL qpLVLlaLLv eiyPlrVtGL VphlrDREKR aipCPQGKYi |
| TNFR1 | | |
| Human | NO:99 | MGLstVPdLL lpLVLleLLv giyPsgViGL VphlgDREKR dsvCPQGKYi |
| TNFR1 | | |
| Pig | NO:101 | MGLstVPgLL lpLVLraLLv dvyPagVhGL VlhpgDREKR eslCPQGKYs |
| TNFR1 | | |
| Consensus | NO:98 | MGL--VP-LL --LVL--LL- ---P--V-GL V----DREKR ---CPQGKY- |
| | | 51 100 |
| Mouse | NO:103 | HsknnSICCT KCHKGTYLvs DCpsPGrdTv CreCekGtFT ASqNylrqCL |
| TNFR1 | | |
| Rat | NO:102 | HpknnSICCT KCHKGTYLvs DCpsPGqeTv CevCdkGtFT ASqNhvrqCL |
| TNFR1 | | |
| Cat | NO:100 | HpqdnSICCT KCHKGTYLyn DCagPGldTd CreCenGtFT ASeNylrqCL |
| TNFR1 | | |
| Human | NO:99 | HpqnnSICCT KCHKGTYLyn DCpgPGqdTd CreCesGsFT ASeNhlrhCL |
| TNFR1 | | |
| Pig | NO:101 | HpqnrSICCT KCHKGTYLhn DClgPGldTd CreCdnGtFT ASeNhltqCL |
| TNFR1 | | |
| Consensus | NO:98 | H----SICCT KCHKGTYL-- DC--PG--T- C--C--G-FT AS-N----CL |
| | | 101 150 |
| Mouse | NO:103 | SCktCRkEMs QVEISpCqad kDTVCGCkeN QfqrYlSEth FQCvdCSpCf |
| TNFR1 | | |
| Rat | NO:102 | SCktCRkEMf QVEISpCkad mDTVCGCkkN QfqrYlSEth FQCvdCSpCf |
| TNFR1 | | |
| Cat | NO:100 | SCskCRkEMy QVEISpCtvy rDTVCGCrkN QyryYwSEth FQClnCSlCl |
| TNFR1 | | |
| Human | NO:99 | SCskCRkEMg QVEISsCtvd rDTVCGCrkN QyrhYwSEnl FQCfnCSlCl |
| TNFR1 | | |
| Pig | NO:101 | SCskCRsEMs QVEISpCtvd rDTVCGCrkN QyrkYwSEtl FQClnCSlCp |
| TNFR1 | | |
| Consensus | NO:98 | SC--CR-EM- QVEIS-C--- -DTVCGC--N Q---Y-SE-- FQC--CS-C- |
| | | 151 200 |
| Mouse | NO:103 | NGTVtipCkE tQnTvCnCHa GFFLresECv pCshCkKnee CmnkLClpppl |
| TNFR1 | | |
| Rat | NO:102 | NGTVtipCkE kQnTvCnCHa GFFLsgnECt pCshCkKnqe CmkLCl.ppv |
| TNFR1 | | |
| Cat | NO:100 | NGTVqisCkE tQnTvCtCHa GFFLrgnECv sCvnCkKnte CtkLCv.piv |
| TNFR1 | | |
| Human | NO:99 | NGTVhlsCqE kQnTvCtCHa GFFLrenECv sCsnCkKsle CtkLCl.pqi |
| TNFR1 | | |
| Pig | NO:101 | NGTVqlpClE kQdTiCnCHs GFFLrdkECv sCvnC.Knad CknLCp.ats |
| TNFR1 | | |
| Consensus | NO:98 | NGTV---C-E -Q-T-C-CH- GFFL---EC- -C--C-K--- C--LC----- |
| | | 201 250 |
| Mouse | NO:103 | anvtnpqDsC TaVLLPLVIl lGlCllsfif isLmcRYprw rpevySIiCr |
| TNFR1 | | |
| Rat | NO:102 | anvtnpqDsC TaVLLPLVIf lGlCllffic isLlcRYpqw rprvySIiCr |
| TNFR1 | | |
| Cat | NO:100 | etvkdpqDpG TtVLLPLVIf fGiCvis.fs igLmcRYqrr ksklfSIvCg |
| TNFR1 | | |
| Human | NO:99 | envkgteDsG TtVLLPLVIf fGlCllsllf igLmyRYqrw ksklySIvCg |
| TNFR1 | | |
| Pig | NO:101 | etrndfqDtG TtVLLPLVIf fGlClafflf vgLacRYqrw kpklySIiCg |
| TNFR1 | | |
| Consensus | NO:98 | -------D-G T-VLLPLVI- -G-C------ --L--RY--- -----SI-C- |
| | | 251 300 |
| Mouse | NO:103 | dpvPvKE.Ek ag...kPltp apspaFSPts gfnPTlgFS. tPgfsspvSs |
| TNFR1 | | |
| Rat | NO:102 | dsapvKEvEg egivtkPltp asipaFSPnp gfnPTlgFSt tPrfshpvSs |
| TNFR1 | | |
| Cat | NO:100 | kstPtKEgE. ....pqp.l. atgpgFSPip ..sPT..FSp sP..tftpSp |
| TNFR1 | | |
| Human | NO:99 | kstPeKEgEl egtttkP.l. apnpsFSPtp gftPTlgFSp vPsstftsSs |
| TNFR1 | | |
| Pig | NO:101 | kstPvKEgEp eplataPsf. gpittFSPip sfsPTttFSp vPsfspisSp |
| TNFR1 | | |
| Consensus | NO:98 | ---P-KE-E- ------P--- -----FSP-- ---PT--FS- -P------S- |
| | | 301 350 |
| Mouse | NO:103 | TpispifgPs nwh.f..mpp vsEvvPt.QG AdPlLyeslc svPaptsvqK |
| TNFR1 | | |
| Rat | NO:102 | TpispvfgPs nwhnf..vpp vrEvvPt.QG AdPlLygsln pvPipapvrK |
| TNFR1 | | |
| Cat | NO:100 | T.....ftPs dwanlraasv srEmaPpyQG AgPiLsaapa ssPistpvqK |
| TNFR1 | | |
| Human | NO:99 | T.....ytPg dcpnf..aap rrEvaPpyQG AdPiLatala sdPipnplqK |
| TNFR1 | | |
| Pig | NO:101 | T.....ftPc dwsnikvtsp pkEiaPppQG AgPiLpmppa stPvptplpK |
| TNFR1 | | |
| Consensus | NO:98 | T-------P- ---------- --E--P--QG A-P-L----- --P------K |
| | | 351 400 |
| Mouse | NO:103 | Wed.....sa hPqrpdnaDl AiLYAVVdgV PPaRWKEFmR fmGLSeHEIe |
| TNFR1 | | |
| Rat | NO:102 | Wed...vvaa qPgrldtaDp AmLYAVVdgV PPtRWKEFmR llGLSeHEIe |
| TNFR1 | | |
| Cat | NO:100 | Wedstht..q rPea.dpaDp AtLYAVVdgV PPsRWKEFvR rlGLSeHEIe |
| TNFR1 | | |
| Human | NO:99 | Wedsah.... kPqsldtdDp AtLYAVVenV PPlRWKEFvR rlGLSdHEId |
| TNFR1 | | |
| Pig | NO:101 | Wggsahsahs aPaqladaDp AtLYAVVdgV PPtRWKEFvR rlGLSeHEIe |
| TNFR1 | | |
| Consensus | NO:98 | W--------- -P------D- A-LYAVV--V PP-RWKEF-R --GLS-HEI- |
| | | 401 450 |
| Mouse | NO:103 | RLEmQNGRCL REAqYSMLea WRRRTpRhEd TLevVGlVLs kMnLaGCLEn |
| TNFR1 | | |
| Rat | NO:102 | RLElQNGRCL REAhYSMLea WRRRTpRhEa TLdvvGrVLc dMnLrGCLEn |
| TNFR1 | | |
| Cat | NO:100 | RLElQNGRCL REAhYSMLaa WRRRTpRrEa TLellGrVLr dMdLlGCLEd |
| TNFR1 | | |
| Human | NO:99 | RLElQNGRCL REAqYSMLat WRRRTpRrEa TLellGrVLr dMdLlGCLEd |
| TNFR1 | | |
| Pig | NO:101 | RLElQNGRCL REAcYSMLae WRRRTsRrEa TLellGsVLr dMdLlGCLEd |
| TNFR1 | | |
| Consensus | NO:98 | RLE-QNGRCL REA-YSML-- WRRRT-R-E- TL---G-VL- -M-L-GCLE- |
| | | 451 469 |
| Mouse | NO:103 | IlEaLrnPAp .ssttrLpR |
| TNFR1 | | |
| Rat | NO:102 | IrEtLesPAh .sstthLpR |
| TNFR1 | | |
| Cat | NO:100 | IeEaLcaPAs lspaprLlR |
| TNFR1 | | |
| Human | NO:99 | IeEaLcgPAa lppapsLlR |
| TNFR1 | | |
| Pig | NO:101 | IeEaLrgPAr lapaphLlR |
| TNFR1 | | |
| Consensus | NO:98 | I-E-L--PA- ------L-R |

TABLE 21
Location of domains within TNFR1 amino acid sequences; numbering refers
to amino acid positions within the corresponding SEQ ID NOs

| | SEQ | | | | | “Mature”^{A} |
|---|---|---|---|---|---|---|
| Polypeptide | ID | Signal | ExtracellularS | Transmembrane | Intracellular | polypeptide |
| Mouse TNFR1 | NO: 103 | 1-21 | 22-212 | 213-235 | 236-454 | 22-454 |
| Rat TNFR1 | NO: 102 | 1-21 | 22-211 | 212-234 | 235-461 | 22-461 |
| Cat TNFR1^{B} | NO: 100 | 1-21 | 22-211 | 212-233 | 234-446 | 22-446 |
| Human TNFR1 | NO: 99 | 1-21 | 22-211 | 212-234 | 235-455 | 22-455 |
| Pig TNFR1 | NO: 101 | 1-21 | 22-210 | 211-233 | 234-461 | 22-461 |
| Consensus | NO: 98 | 1-21 | 22-212 | 213-235 | 236-469 | 22-469 |

^{A}For the purposes of the above table, the “mature” polypeptide refers to a polypeptide from which the indicated signal sequence has been cleaved; additional mature polypeptide forms may occur.
^{B}The positions of domains within the cat TNFR1 amino acid sequence have been determined by comparison with the corresponding domains of the other mammalian TNFR1 amino acid sequences.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for detecting the presence of a compound in a sample, comprising:
(a) providing, in any order:
(i) a sample suspected of comprising a compound and a control sample without the compound;
(ii) a receptor and a response gene; and
(iii) a ligand, wherein the ligand is capable of binding the receptor, thereby altering the expression of the response gene;
(b) combining, in any order, (i) the sample, the receptor, and the ligand;
and (ii) the control sample, the receptor and the ligand; and
(c) measuring the level of the expression of the response gene;
wherein the presence of the compound in the sample is detected by an alteration in the level of expression of the response gene when compared to the level of expression of the response gene when the receptor is combined with the ligand in the presence of the control sample.

2. A method for detecting the presence of a compound in the presence or absence of a sample, comprising:
(a) providing, in any order:
(i) a compound, wherein the compound is in the presence or absence of a sample;
(ii) a receptor and a response gene; and
(iii) a ligand, wherein the ligand is capable of binding the receptor, thereby altering the expression of the response gene;
(b) combining, in any order, (i) the compound, the receptor, and the ligand; and (ii) the receptor and the ligand; and
(c) measuring the level of the expression of the response gene, wherein the presence of the compound is measured by an alteration in the level of expression of the response gene when the receptor is combined with the ligand and the compound compared to the level of expression of the response gene when the receptor is combined with the ligand only; and wherein when the receptor is combined with varying concentrations of the ligand and the compound, the expression of the response gene in the presence of the sample is correlated with the expression of the response gene in the absence of the sample with a correlation coefficient of at least 0.5.

3. The method of claim 1, wherein the ligand is a therapeutic substance for administration to a subject.

4. The method of claim 3, wherein the compound is a neutralizing antibody against the therapeutic substance.

5. The method of any one of claims 1-4, wherein the receptor comprises SEQ ID NO:1.

6. The method of any one of claims 1-4, wherein the receptor comprises SEQ ID NO:2, SEQ ID NO:3, SEQ ID NO:4, or SEQ ID NO:5.

7. The method of claim 5, wherein the therapeutic substance comprises SEQ ID NO:6.

8. The method of claim 6, wherein the therapeutic substance comprises SEQ ID NO:6.

9. The method of claim 5, wherein the therapeutic substance comprises SEQ ID NO:7, SEQ ID NO:8, SEQ ID NO:9, SEQ ID NO:10, SEQ ID NO:11, SEQ ID NO:12, SEQ ID NO:13, or SEQ ID NO:14.

10. The method of claim 6, wherein the therapeutic substance comprises SEQ ID NO:7, SEQ ID NO:8, SEQ ID NO:9, SEQ ID NO:10, SEQ ID NO:11, SEQ ID NO:12, SEQ ID NO:13, or SEQ ID NO:14.

11. The method of claim 5, wherein the response gene comprises SEQ ID NO:15.

12. The method of claim 6, wherein the response gene comprises SEQ ID NO:16, SEQ ID NO:17, SEQ ID NO:18, SEQ ID NO:19, SEQ ID NO:20, or SEQ ID NO:21.

13. The method of any one of claims 1-4, wherein the receptor comprises the extracellular domain of SEQ ID NO:80.

14. The method of any one of claims 1-4, wherein the receptor comprises the extracellular domain of SEQ ID NO:81, SEQ ID NO:82, or SEQ ID NO:83.

15. The method of claim 13, wherein the ligand comprises SEQ ID NO:84.

16. The method of claim 14, wherein the ligand comprises SEQ ID NO:84.

17. The method of claim 13, wherein the ligand comprises SEQ ID NO:85, SEQ ID NO:86, SEQ ID NO:87, SEQ ID NO:88, SEQ ID NO:89, SEQ ID NO:90, or SEQ ID NO:91.

18. The method of claim 14, wherein the ligand comprises SEQ ID NO:85, SEQ ID NO:86, SEQ ID NO:87, SEQ ID NO:88, SEQ ID NO:89, SEQ ID NO:90, or SEQ ID NO:91.

19. The method of claim 13, wherein the response gene comprises SEQ ID NO:15.

20. The method of claim 14, wherein the response gene comprises SEQ ID NO:15.

21. The method of any one of claims 1-4, wherein the receptor comprises the extracellular domain of SEQ ID NO:92.

22. The method of any one of claims 1-4, wherein the receptor comprises the extracellular domain of SEQ ID NO:93 or SEQ ID NO:94.

23. The method of claim 21, wherein the ligand comprises SEQ ID NO:95.

24. The method of claim 22, wherein the ligand comprises SEQ ID NO:95.

25. The method of claim 21, wherein the ligand comprises SEQ ID NO:96 or SEQ ID NO:97.

26. The method of claim 22, wherein the ligand comprises SEQ ID NO:96 or SEQ ID NO:97.

27. The method of claim 21, wherein the response gene comprises SEQ ID NO:98.

28. The method of claim 22, wherein the response gene comprises SEQ ID NO:98.

28. The method of claim 21, wherein the response gene comprises SEQ ID NO:99, SEQ ID NO:100, SEQ ID NO:101, SEQ ID NO:102, or SEQ ID NO:103.

29. The method of claim 23, wherein the response gene comprises SEQ ID NO:99, SEQ ID NO:100, SEQ ID NO:101, SEQ ID NO:102, or SEQ ID NO:103.

30. The method of claim 21, wherein the response gene comprises SEQ ID NO:15.

31. The method of claim 22, wherein the response gene comprises SEQ ID NO:15.

32. The method of claim 23, wherein the response gene comprises SEQ ID NO:15.

33. The method of claim 24, wherein the response gene comprises SEQ ID NO:15.

34. The method of any one of claims 1-4, wherein the ligand comprises SEQ ID NO:105, SEQ ID NO:106, or SEQ ID NO:107.

35. The method of claim 34, wherein the receptor comprises SEQ ID NO:108 or SEQ ID NO:109.

36. The method of claim 34, wherein the response gene is tartrate resistant acid phosphatase (TRAP).

37. The method of any one of claims 1-4, wherein the ligand is an endogenous ligand, which is bound by a therapeutic substance for administration to a subject.

38. The method of any one of claims 1-4, wherein the level of the expression of the response gene is measured using a bDNA assay.

39. The method of any of the claims 1-4, wherein the sample is selected from the group consisting of whole blood, plasma, serum, synovial fluid, ascitic fluid, lacrimal fluid, perspiration, seminal fluid, cell extracts, and tissue extracts.

40. The method any one of claims 1-4, wherein the receptor is expressed by a mammalian cell.

41. A kit comprising (a) a cell expressing a receptor, wherein the receptor comprises the intracellular domain of EPOR, and (b) one or more oligonucleotides used to detect PIM 1 gene expression, the oligonucleotides selected from the group consisting of SEQ ID NOs:22 through 79.
